(19) United States
(12) Patent Application Publication
(10) Pub. No.: US 2011/0086813 A1
(43) Pub. Date: Apr. 14, 2011

(54) COMPOUNDS FOR TREATING BACTERIAL INFECTIONS

(76) Inventors: Gad Glaser, Jerusalem (IL); Jehoshua Katzhendler, Jerusalem (IL); Rolf Hilgenfeld, Luebeck (DE); Roee Vidavski, Rehovot (IL); Ezequiel Wexselblatt, Beit Zayit (IL); Tamar Prez-Menahemov, Jerusalem (IL); Ilana Kaspy, Givat Zeev (IL)

(21) Appl. No.: 12/933,658

(22) PCT Filed: Mar. 18, 2009
(86) PCT No.: PCT/IL09/00309

§ 371 Date: Dec. 9, 2010

Related U.S. Application Data
(60) Provisional application No. 61/037,952, filed on Mar. 19, 2008.

(51) Int. Cl. A61K 31/708; C07H 19/04; A61P 31/04

(52) U.S. Cl. 514/48; 536/26.2; 536/26.22

(57) ABSTRACT

The present invention relates to a novel class of guanine nucleotide analogs which inhibit RelA and Relseq synthetic activity and which possess anti-bacterial activity. The present invention also relates to pharmaceutical compositions that include such compounds, and to methods of use of such compounds or compositions for combating bacteria and treating bacterial infections.

### FIELD OF THE INVENTION

The present invention relates to a novel class of guanine nucleotide analogs, which inhibit RelA and Relseq synthetic activity and possess anti-bacterial activity, to pharmaceutical compositions comprising such compounds, and to methods of use thereof for combating bacteria and treating bacterial infections.

### BACKGROUND IN THE INVENTION

The natural environment of bacteria is often characterized by changes in nutrient availability. When bacterial cells are deprived of an amino acid or carbon source, changes in many cellular processes occur. This pleiotropic response, called the stringent response, was initially described for Escherichia coli in 1961. The first observed feature of the stringent response was the intracellular accumulation of two unusual phosphorylated derivatives of GTP and GDP (collectively termed (p)ppGpp), within a few seconds after amino acid starvation (Cashel and Gallant, 1969; Cashel et al., 1969). Other features of the stringent response include inhibition of rRNA and tRNA synthesis, inhibition of replication initiation and cell division, suppression of the active transport of many metabolites, transcriptional upregulation of genes encoding enzymes involved in amino acid biosynthesis (Cashel, 1996), and induction of the rpoS gene, which encodes the stationary phase sigma factor (Gentry et al., 1993).

The major effector of the stringent response is most likely (p)ppGpp. In E. coli, the mutation causing the relaxed phenotype, which fails to accumulate (p)ppGpp during amino acid starvation, was mapped to the relA gene which encodes an 84 kDa protein, RelA (Metzger et al., 1988). The RelA protein is a ribosome-associated (p)ppGpp synthetase that is activated in response to amino acid starvation. Synthesis of (p)ppGpp has been characterized as a pyrophosphoryl group transfer of the β and γ phosphates from an ATP donor to the ribose 3′ hydroxyl of GTP (or GDP) (Cashel, 1996). For this reaction to occur in vitro, purified RelA requires mRNA, functional ribosomes paused during elongation at a ‘hungry codon’, and uncharged cognate tRNA bound at the acceptor site of that hungry codon (Cashel, 1996). In cell extracts, RelA is found associated to only a small fraction of the ribosomes (about 1%) (Pedersen and Kjeldgaard, 1977). RelA is thus a ribosome-dependent enzyme that senses environmental amino acid levels by monitoring the amount of uncharged tRNA present in the cell, and accordingly synthesizes the intracellular second-messenger, (p)ppGpp (Haseltine, 1973; Metzger et al., 1988).

In addition to RelA, a second gene product, SpoT, is involved in (p)ppGpp metabolism in E. coli. SpoT is a cytosolic protein that functions as a (p)ppGpp synthetase upon carbon or fatty acid limitation (Gentry and Cashel, 1995; Metzger et al., 1989a; Seyfzadeh et al., 1993). Equally important, SpoT also acts as a ribosome-independent (p)ppGpp hydrolase that degrades the (p)ppGpp back to GDP(GTP) and pyrophosphate, thus catalyzing a reaction opposing the synthesis of (p)ppGpp from GDP(GTP) and ATP (Metzger et al., 1989a). Residual (p)ppGpp synthesis found in a ΔrelA mutant (relA1) is abolished in a ΔrelAΔspoT (“double null”) mutant (Xiao et al., 1991). Cells with this double deletion show a complex phenotype, such as loss of ability to grow on amino acid-free minimal medium, morphological alterations and more (Xiao et al., 1991).

It has been found that in several Gram-positive bacteria, only one relA/spoT paralog exists which is capable of carrying out both the (p)ppGpp-synthetase and (p)ppGpp-hydrolase functions (Mechold et al., 1996; Mechold and Malke, 1997; Mittenhuber, 2001; Wendrich and Marahiel, 1997). Deletion of this one gene in gram-positive bacteria creates a phenotype resembling that of the ‘double null’ E. coli cells (Wendrich and Marahiel, 1997). This bifunctional gene product was even found to be essential in the highly pathogenic Staphyloccous aureus (Gentry et al., 2000). The gram-negative Myxococcus xanthus has both relA and spoT analogs, which appear to be involved in fruiting-body development and spore formation in response to starvation (Harris et al., 1998). Rel/Spo genes are absent in Archaea, in agreement with the transcriptional system being closer to that of eukaryotes, but they are again found in the genome of plants, e.g. Arabidopsis thaliana, where they play a role in activating a (p)ppGpp-mediated stress response (van der Biezen et al., 2000; Givens et al., 2004; Takahashi et al., 2004).

The crystal structure of the N terminal domain (NTD) of Rel/Spo from Streptococcus equisimilis (Relseq) reveals two enzyme conformations. This domain has two sub-domains, each with a catalytic site, one responsible for the synthesis of (p)ppGpp, the other for its hydrolysis. The X-ray structure of the NTD also revealed the binding sites for two guanosine nucleotides.

There is an ongoing and unmet need in the art to identify new compounds acting as anti-bacterial agents. In addition, there is a need to combat the growing problem of bacterial resistance to anti-bacterial agents.

RelA was found to be involved in the virulence, biofilm formation and survival of many bacteria species. Because RelA and its homologues are completely absent in mammals, new antibacterial compounds could be designed based on the known X-ray structure of the NTD of Relseq.

### SUMMARY OF THE INVENTION

The present invention is based on the discovery of a novel class of compounds which display activity against a wide range of bacteria. As contemplated herein, the inventors of the present application designed a group of guanine nucleotide analogs, which inhibit RelA and Relseq synthetic activity and which possess anti-bacterial activity. The present invention also relates to pharmaceutical compositions comprising such compounds, and to methods of use thereof for combating bacteria and treating bacterial infections.

Without wishing to be bound by any particular mechanism or theory, it is contemplated that, by inhibiting RelA and Relseq synthetic activity, bacteria are prevented from sensing the lack of amino acids in their habitat. This will result in the bacteria not reacting to the changes in their environment, which will ultimately lead to their starvation and death. This mechanism differs from other, frequently used antibacterial compounds in the way that it does not cause selective pressure on the bacteria and therefore will not lead the bacteria to look for alternative pathways to survive.

In one embodiment, the compounds are represented by formula (I), as described herein.

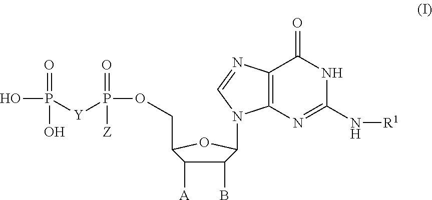

wherein:
A and B are independently selected from the group consisting of:

(a) —H;

(b) —OR²;

(c)

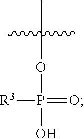

(d)

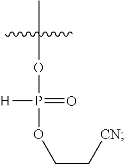

(e) —NR⁴R⁵;

(f) N₃;

(g)

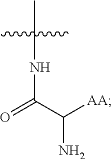

(h)

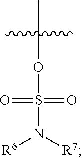

and

(i)

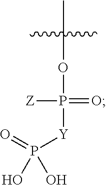

or A and B together represent a moiety selected from:

(a)

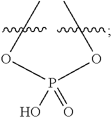

(b)

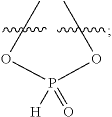

and

(c)

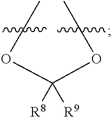

### Y is CH₂ or O;

Z is selected from the group consisting of:

(a) OH;

(b)

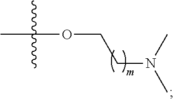

(c)

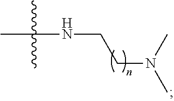

(d)

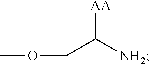

(e)

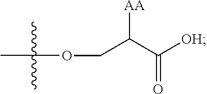

and

(f)

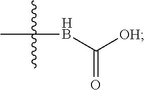

R¹ is H, —COR¹⁰ or an amino protecting group;
R² is H, C₁-C₄ alkyl or a hydroxyl protecting group;
R³ is selected from the group consisting of:

(a) —H;

(b) —OH;

(c)

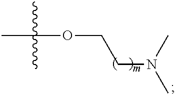

(d)

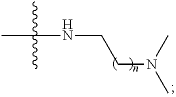

(e)

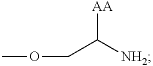

(f)

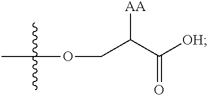

and

(g)

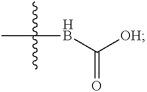

R⁴ and R⁵ are independently H, C₁-C₄ alkyl or an amino protecting group;
R⁶ and R⁷ are independently selected from the group consisting of:

(a) —H;

(b) C₁-C₄ alkyl;

(c) an amino protecting group; and

(d)

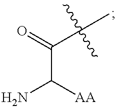

R⁸ and R⁹ are independently selected from the group consisting of:

(a) H;

(b) C₁-C₄ alkyl;

(c) unsubstituted or substituted aryl, heteroaryl, cycloalkyl or heterocyclyl; and

(d) —(CH₂)ₚ—COOH;

R¹⁰ is H or a C₁-C₄ alkyl;
m, n and p are each independently selected from 0, 1, 2, 3, 4, 5 and 6; and
AA represents an amino acid side chain;
with the proviso that:

(a) when Y is O; Z is OH and R¹ is H:
(i) A and B are not both H or OH;
(ii) when one of A and B is H, the other one is not OH;
(iii) when A is

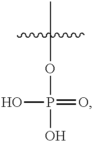

### B is not H or OH;

(iv) when B is

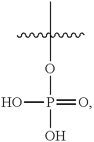

### A is not OH;

(v) when A is

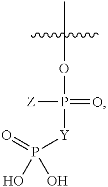

then B is not H or OH;
(vi) when B is

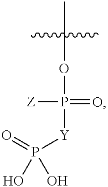

then A is not OH;
(vii) A and B together are not

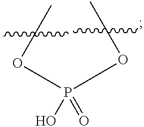

(viii) when A is NH₂, B is not OH; and
(ix) when A is N₃, B is not H;

(b) when Y is CH₂; Z is OH and R¹ is H:
(i) when one of A and B is

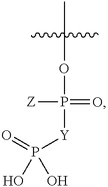

the other is not OH;
(ii) A and B together are not

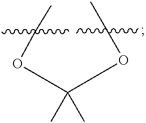

(iii) A and B are not both OH; and
(iv) when B is OH, A is not OCH₃;

including salts, hydrates, solvates, polymorphs, optical isomers, geometrical isomers, enantiomers, diastereomers, complexes and mixtures thereof.

In one preferred embodiment, Y is CH₂. In another preferred embodiment, Y is O. In yet another preferred embodiment, R¹ is H.

Alternatively, in one embodiment, A and B are independently selected from the group consisting of:

(a) —H;

(b) —OR²;

(c)

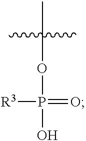

and

(d)

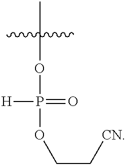

In accordance with this embodiment, the compound may be a compound of Group A (3′(2′) phosphate derivatives), for example a compound selected from the group consisting of any of formulae A2, A3a, A4, A5, A6, A7a, A7b, A7c, A8 and A9, as depicted below.

### Group A: 3′(2′) Phosphate Derivatives

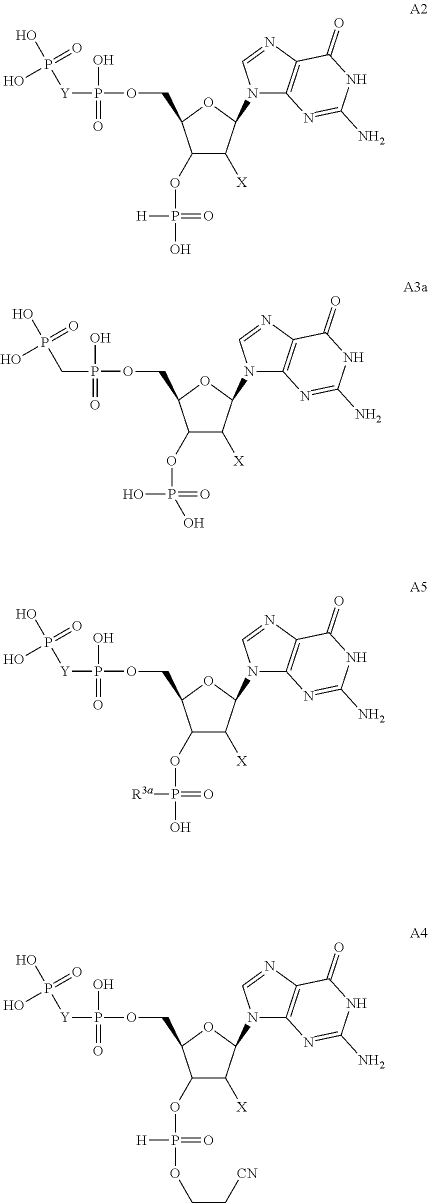

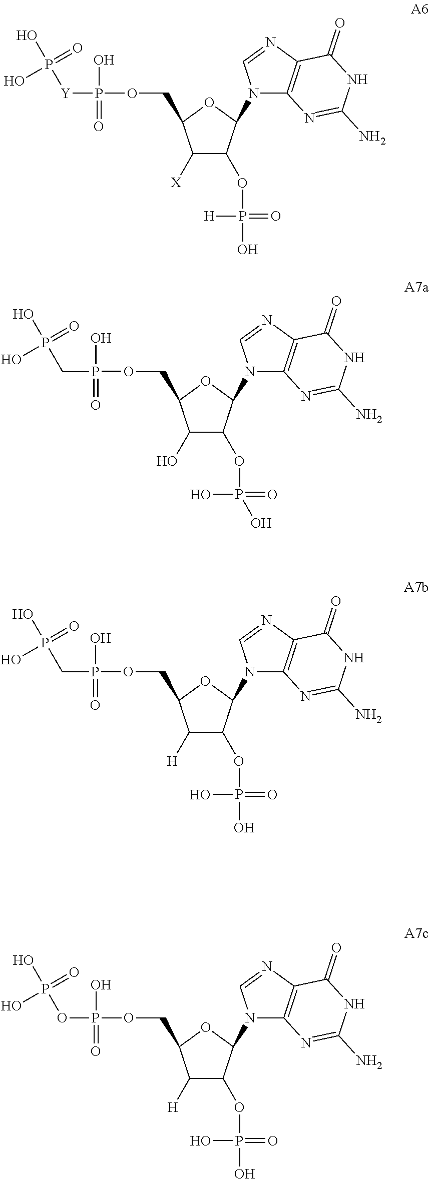

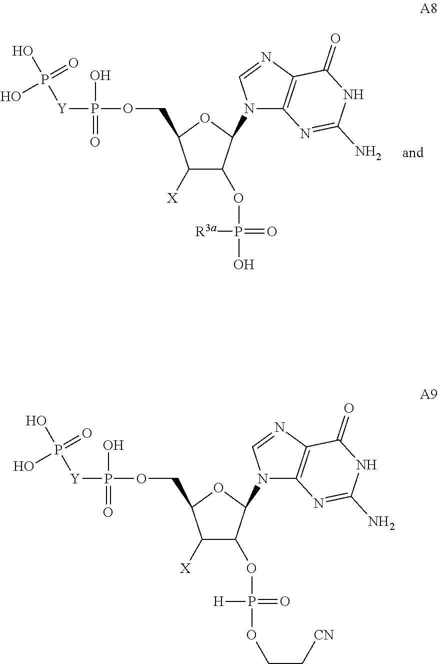

wherein Y is CH₂ or O; X is H or OH; and

R^{3a} is selected from the group consisting of

(a)

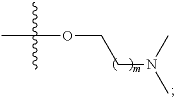

(b)

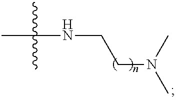

(c)

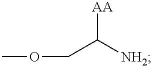

(d)

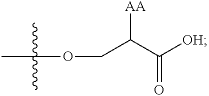

and

(e)

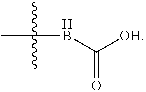

Alternatively, in another embodiment, A and B are independently selected from the group consisting of:

(a) H;

(b) OR²;

(c) —NR⁴R⁵;

(d) N₃; and

(e)

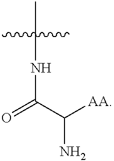

In accordance with this embodiment, the compound may be a compound of Group B (3′ (2′) amine/azide/amino acid derivatives), for example a compound selected from the group consisting of any of formulae B1a, B1b, B1c, B2, B3a, B3b, B3c, B4, B5 and B6, as depicted below.

### Group B: 3′ (2′) Amine/Azide/Amino Acid Derivatives

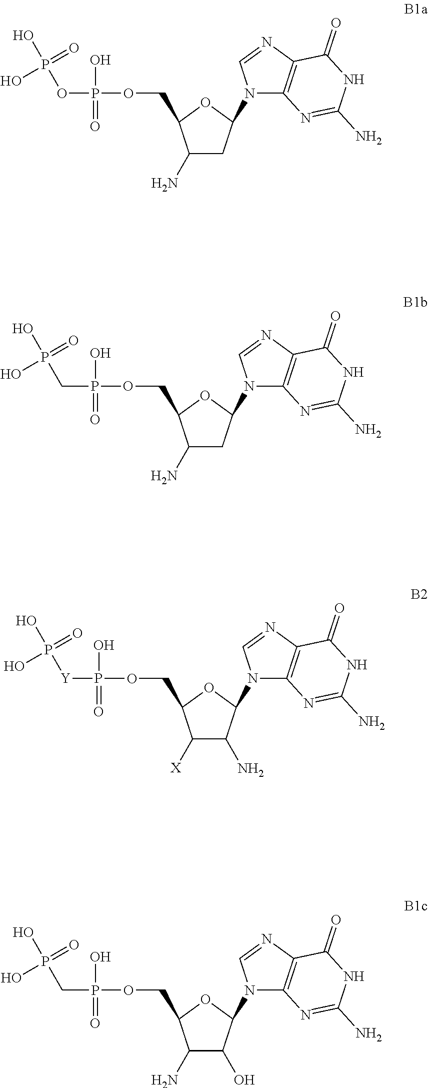

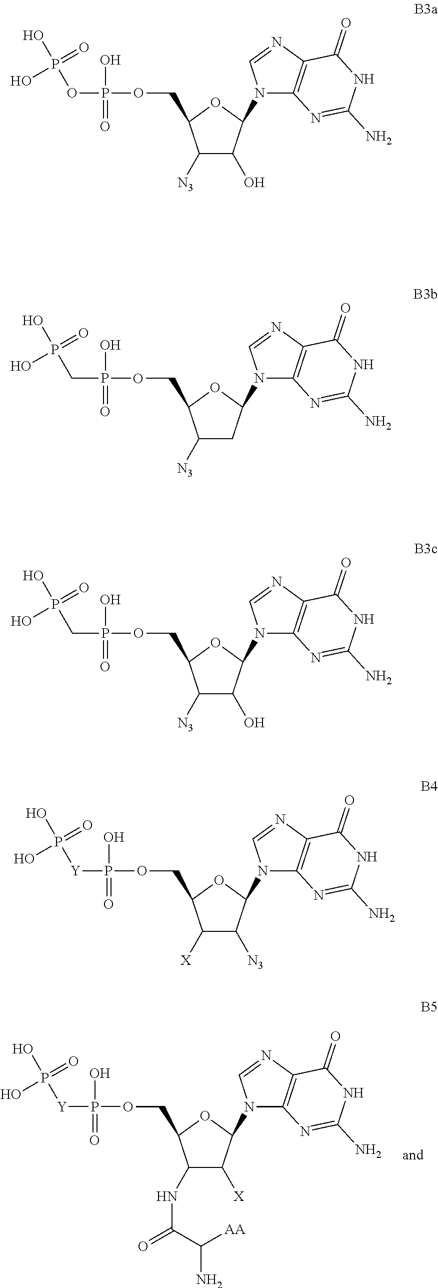

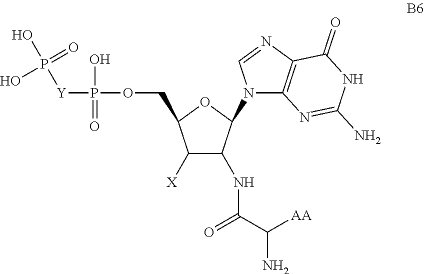

wherein Y is CH₂ or O; X is H or OH; and AA represents an amino acid side chain.

Alternatively, in another embodiment, A and B are independently selected from the group consisting of:

(a) H;

(b) OR²; and

(c)

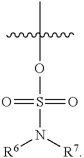

In accordance with this embodiment, the compound may be a compound of Group C (2′/3′ sulfamic acid derivatives), for example a compound selected from the group consisting of any of formulae C1, C2, C3, C4, C5 and C6 as depicted below.

### Group C: 2′/3′ Sulfamic Acid Derivatives

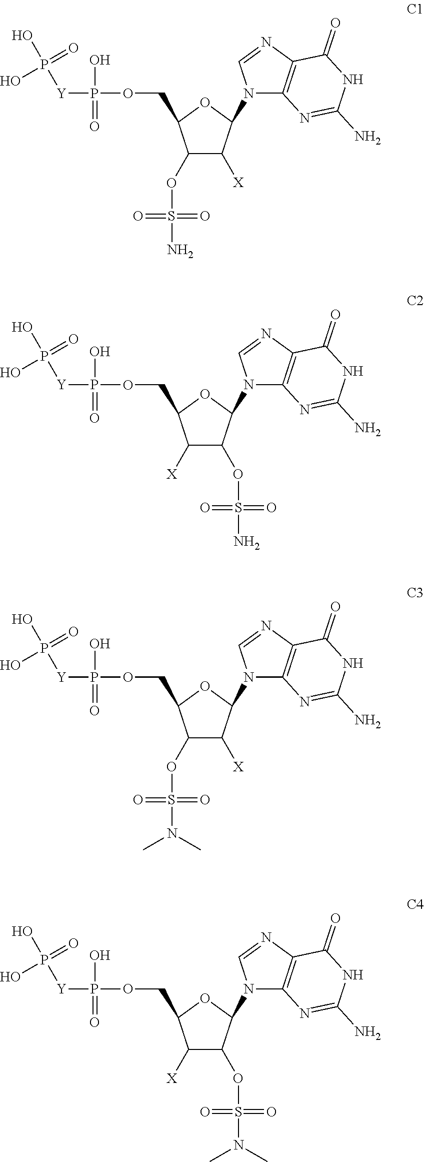

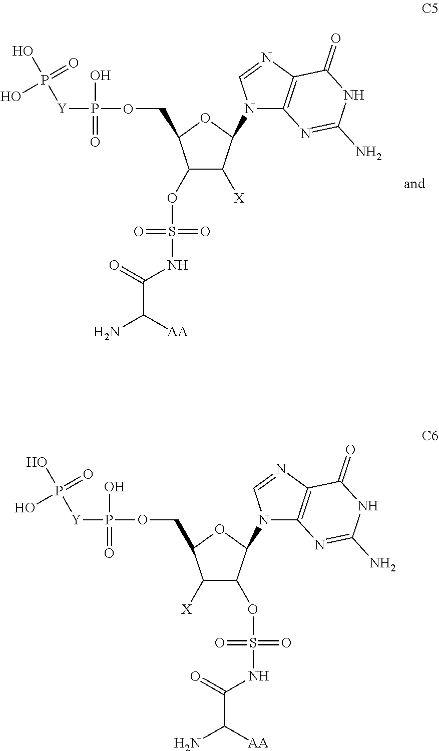

wherein Y is CH₂ or O; X is H or OH; and AA represents an amino acid side chain.

Alternatively, in another embodiment, A and B are independently selected from the group consisting of:

(a) H;

(b) OR²; and

(c)

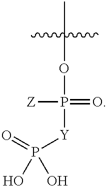

In accordance with this embodiment, the compound may be a compound of Group D (ppGpp analogs), for example a compound selected from the group consisting of any of formulae D1, D2, D3, D4, D5, D6, D7 and D8 as depicted below.

Group D: ppGpp Analogs:

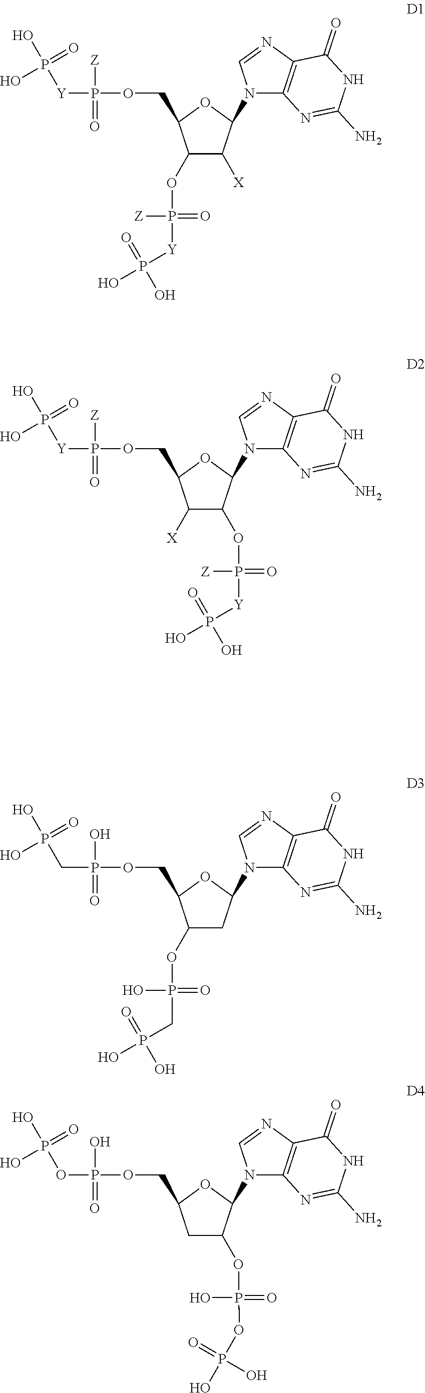

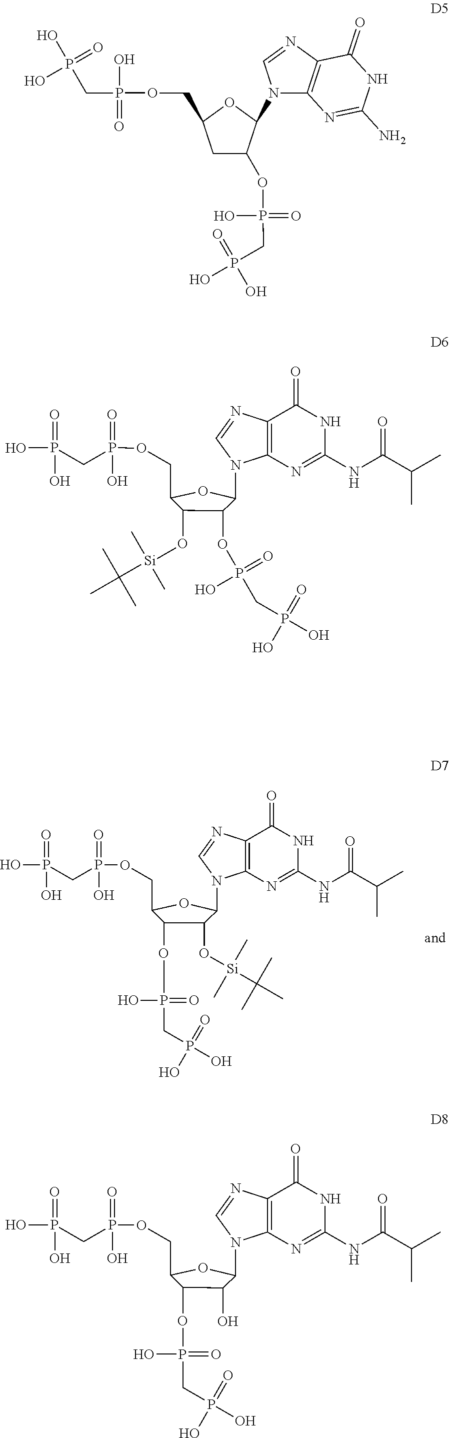

wherein Y is CH₂ or O; X is H or OH; and
Z is selected from the group consisting of:

(a)

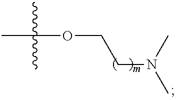

(b)

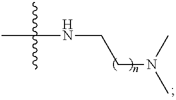

(c)

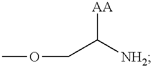

(d)

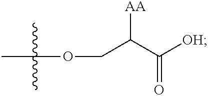

and

(e)

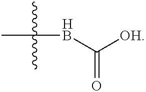

Alternatively, in another embodiment, A and B are independently selected from the group consisting of:

(a)

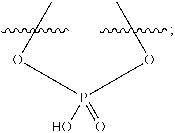

(b)

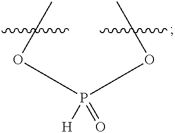

and

(C)

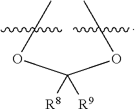

In accordance with this embodiment, the compound may be a compound of Group E (2′3′ cyclic derivatives), for example a compound selected from the group consisting of any of formulae E1, E2, E3 a, E4, E5 and E6 as depicted below.

### Group E: Cyclic Derivatives

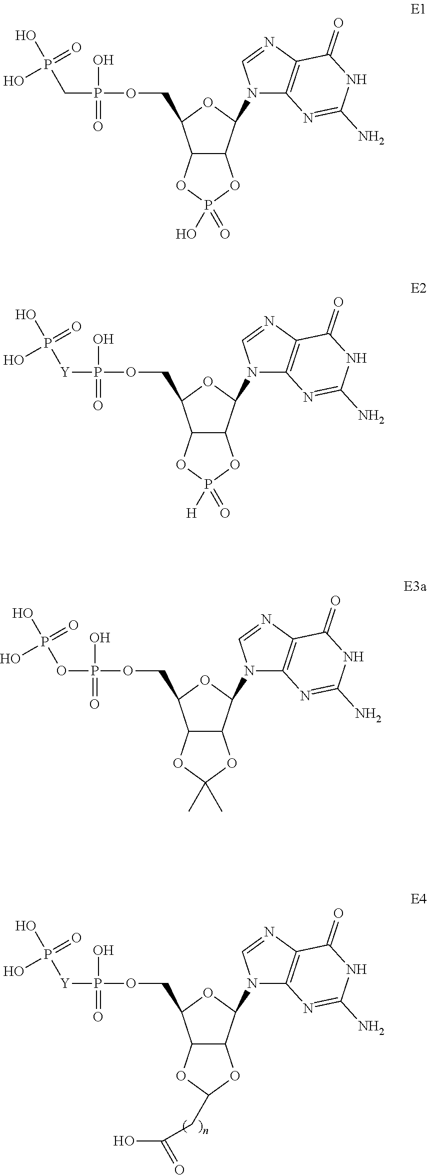

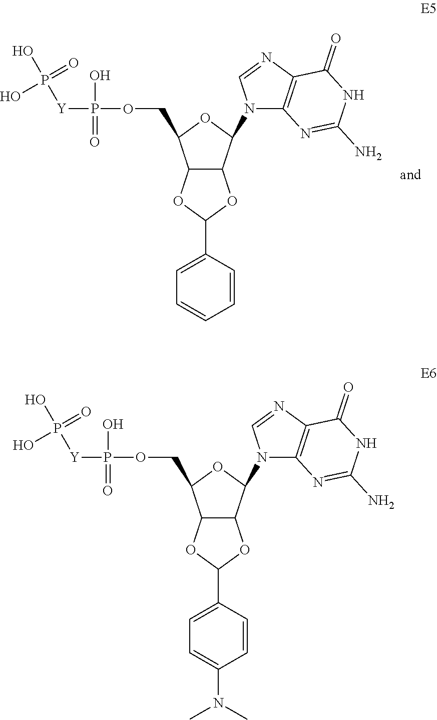

wherein Y is CH₂ or O.

Advantageously, the compound may be selected from the group consisting of:

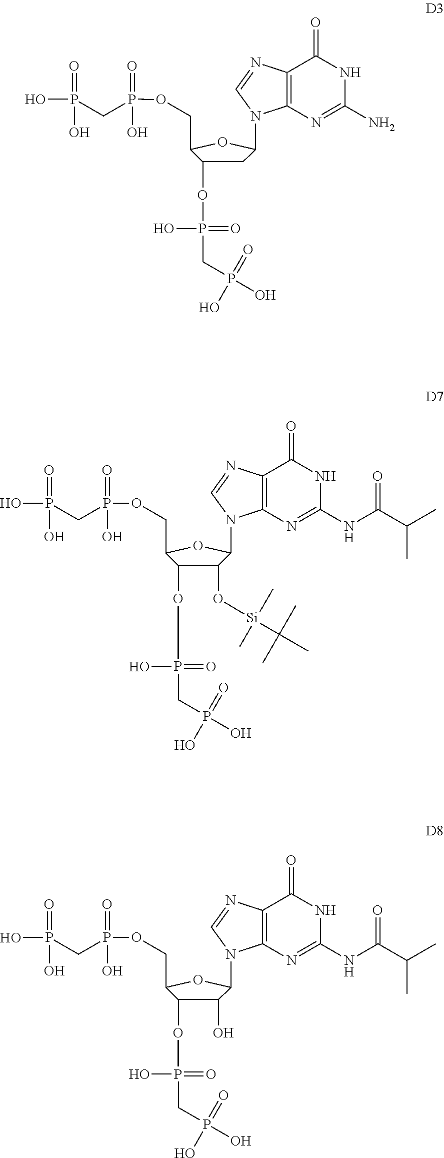

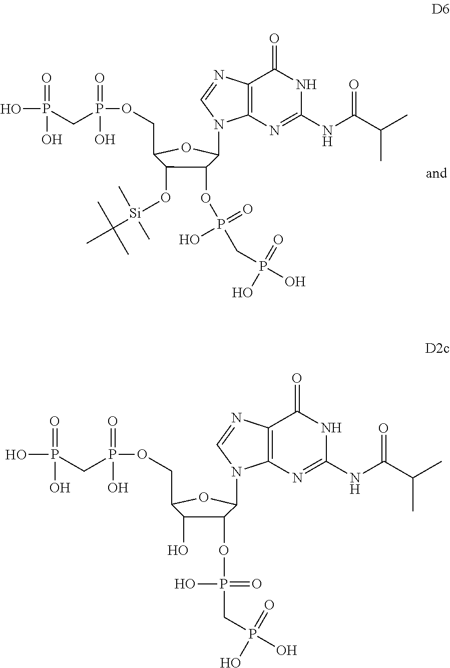

In another aspect, the present invention is based on the finding and realization that compounds of formula (I), and in particular of Groups A-E as described above, can be active as antibacterial agents. Therefore, in another embodiment, the present invention encompasses a pharmaceutical composition comprising a pharmaceutically acceptable carrier or excipient and as an active ingredient a therapeutically effective amount of a compound of formula (I) or of any one of Groups A-E, for example compounds of formulae A2, A3 a, A4, A5, A6, A7a, A1b, A7c, A8, A9, B1a, B1b, B1c, B2, B3a, B3b, B3c, B4, B5, B6, C1, C2, C3, C4, C5, C6, D1, D2, D3, D4, D5, D6, D7, D8, E1, E2, E3a, E4, E5 and E6 as described herein, or complexes of the aforementioned compounds with negative charge neutralizing agents. Preferably the above compositions are anti-bacterial compositions.

Alternatively, the anti-bacterial pharmaceutical compositions of the present invention comprise a therapeutically effective amount of a compound of formula (I-a), and a pharmaceutically acceptable carrier or excipient

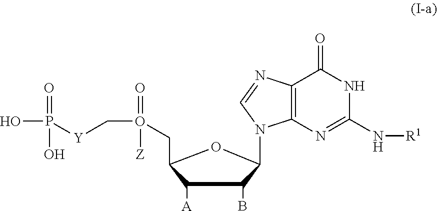

wherein:
A and B are independently selected from the group consisting of:

(a) —H;

(b) —OR²;

(c)

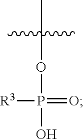

(d)

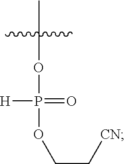

(e) —NR⁴R⁵;

(f) N₃;

(g)

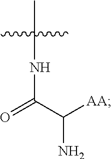

(h)

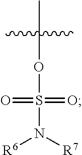

and

(i)

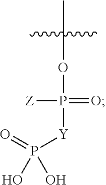

or A and B together represent a moiety selected from:

(a)

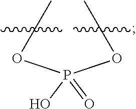

(b)

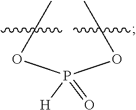

and

(c)

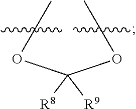

### Y is CH₂ or O;

Z is selected from the group consisting of:

(a) OH;

(b)

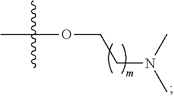

(c)

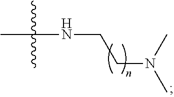

(d)

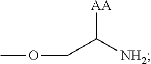

(e)

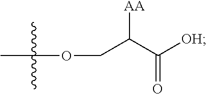

and

(f)

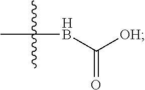

### R¹ is H or —COR¹⁰;

R² is H, C₁-C₄ alkyl or a hydroxyl protecting group;
R³ is selected from the group consisting of:

(a) —H;

(b) —OH;

(c)

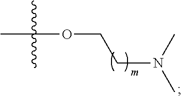

(d)

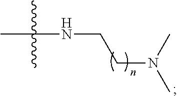

(e)

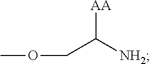

(f)

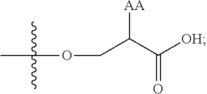

and

(g)

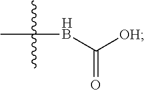

R⁴ and R⁵ are independently H, C₁-C₄ alkyl or an amino protecting group;
R⁶ and R⁷ are independently selected from the group consisting of:

(a) —H;

(b) C₁-C₄ alkyl; and

(c)

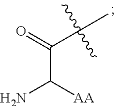

R⁸ and R⁹ are independently selected from the group consisting of:

(a) H;

(b) C₁-C₄ alkyl;

(c) unsubstituted or substituted aryl, heteroaryl, cycloalkyl or heterocyclyl; and

(d) —(CH₂)ₚ—COOH

R¹⁰ is a C₁-C₄ alkyl;
m, n and p are each independently selected from 0, 1, 2, 3, 4, 5 and 6; and
AA represents an amino acid side chain;
including salts, hydrates, solvates, polymorphs, optical isomers, geometrical isomers, enantiomers, diastereomers, complexes and mixtures thereof.

In one embodiment, compound may be a compound of Group A′ (3′(2′) phosphates), such as a compound selected from the group consisting of:

Group A′: 3′(2′) Phosphate Derivatives

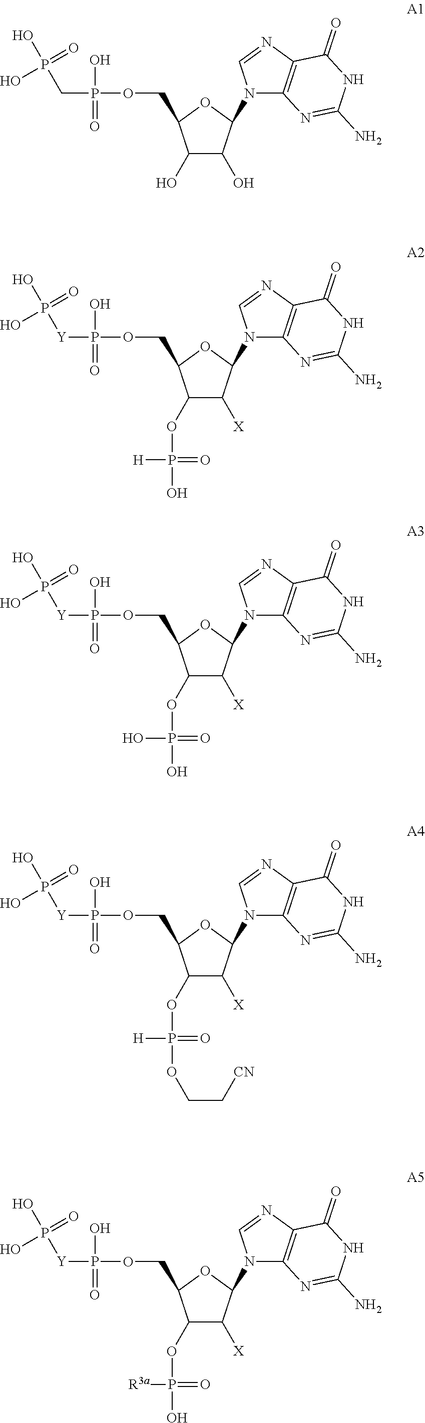

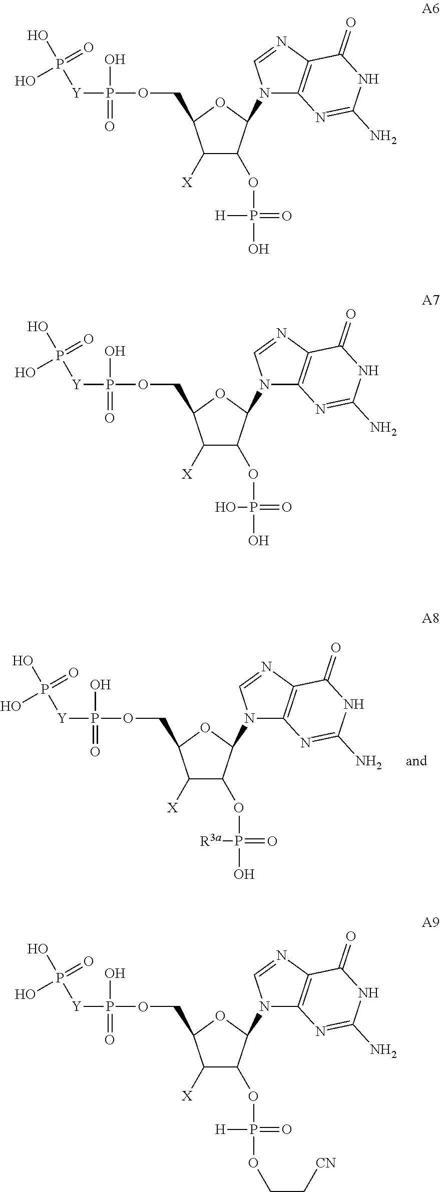

wherein Y is CH₂ or O; X is H or OH; and

R^{3a} is selected from the group consisting of:

(a)

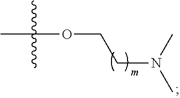

(b)

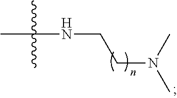

(c)

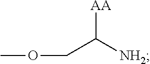

(d)

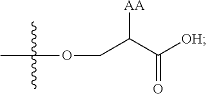

and

(e)

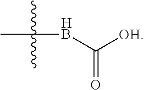

In another embodiment, compound may be a compound of Group B′ (3′(2′) amine/azide/amino acid derivatives), such as a compound selected from the group consisting of:

Group B′: 3′(2′) Amine/Azide/Amino Acid Derivatives

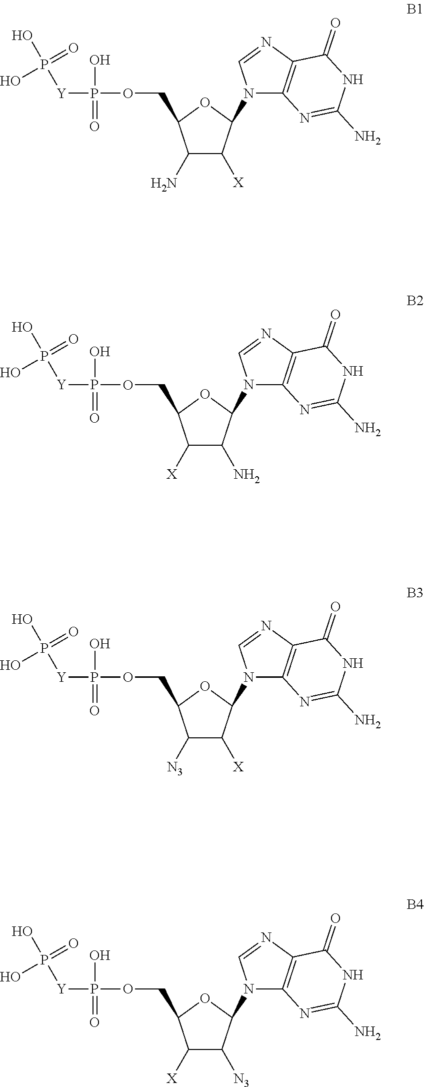

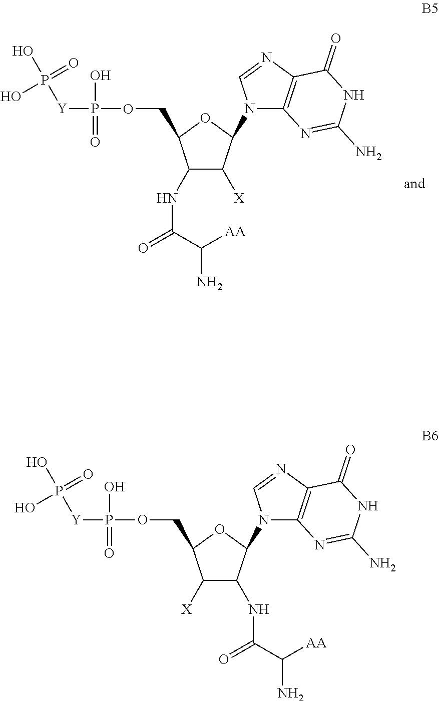

wherein Y is CH₂ or O; X is H or OH; and AA represents an amino acid side chain.

In another embodiment, compound may be a compound of Group C′ (3′(2′) sulfamic acid derivatives), such as a compound selected from the group consisting of:

Group C′: 3′(2′) Sulfamic Acid Derivatives

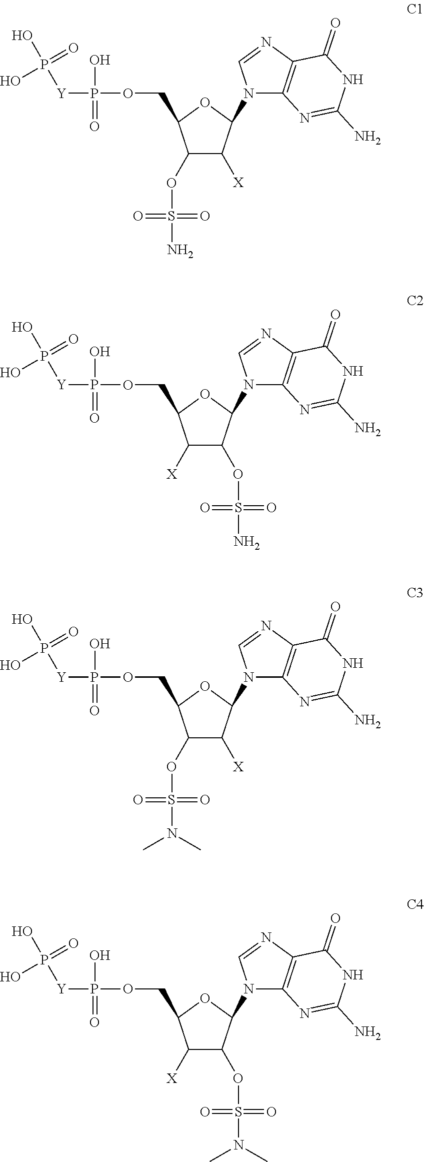

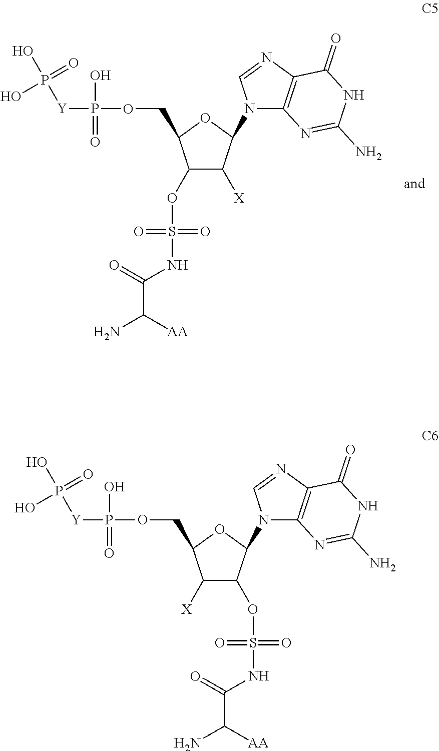

wherein Y is CH₂ or O; X is H or OH; and AA represents an amino acid side chain.

In another embodiment, compound may be a compound of Group D′ (ppGpp analogs), such as a compound selected from the group consisting of:

Group D′: ppGpp Analogs

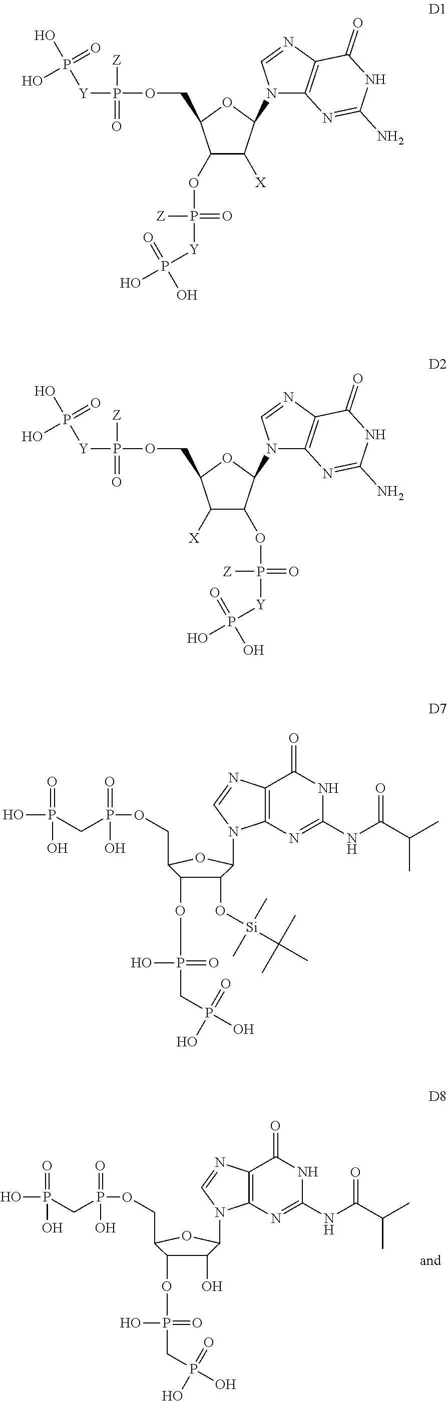

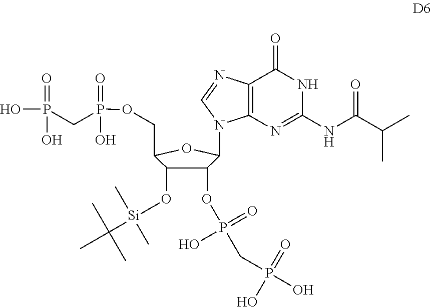

herein Y is CH₂ or O; X is H or OH; and
Z is selected from the group consisting of:

(a) OH;

(b)

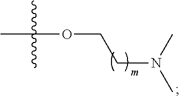

(c)

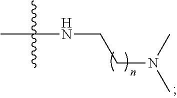

(d)

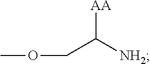

(e)

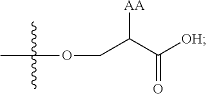

and

(f)

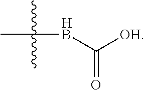

In another embodiment, compound may be a compound of Group E′ (cyclic derivatives), such as a compound selected from the group consisting of Group E′: Cyclic derivatives

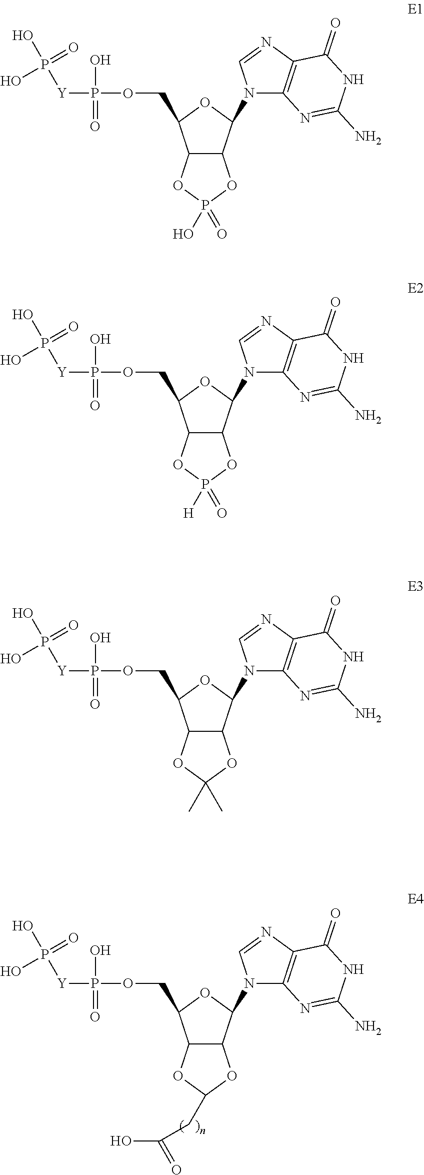

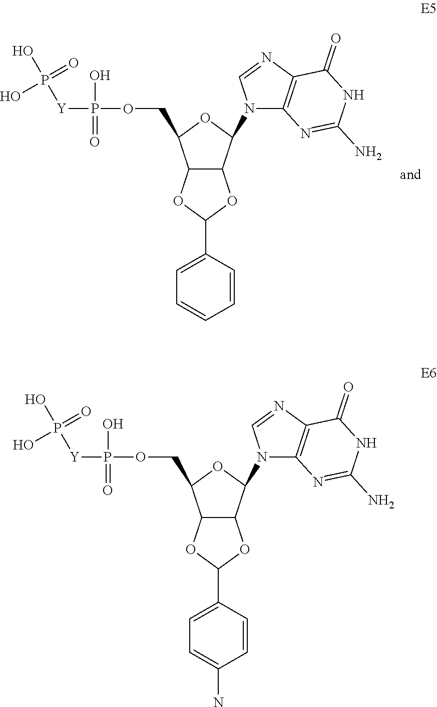

wherein Y is CH₂ or O.

In another aspect, the present invention concerns complexes of the compounds of the present invention with “negative charge neutralizing agents”—i.e., agents that when in association with the compounds of formula (I) or (I-a), or compounds of Groups A-E or A′-E′ results in either a neutral or a positively charged complex that can easily penetrate through the bacterial membrane. Non-limiting examples of such agents being polyamines, esterifying agents, phosphoramidating agents, phosphoboronating agents

Advantageously, the present invention relates to a pharmaceutical composition comprising a pharmaceutically acceptable carrier or excipient, and a therapeutically effective amount of a compound of a formula selected from the group consisting of:

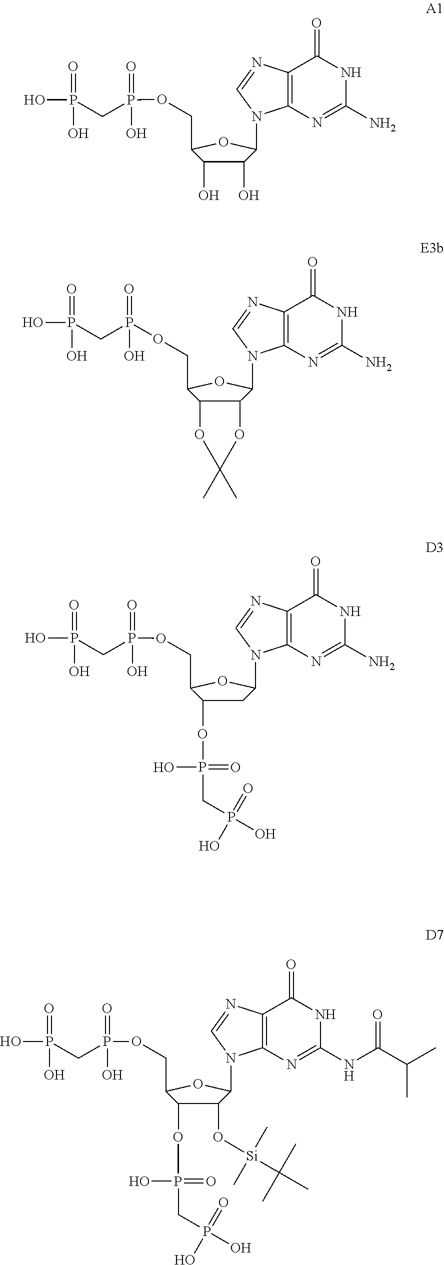

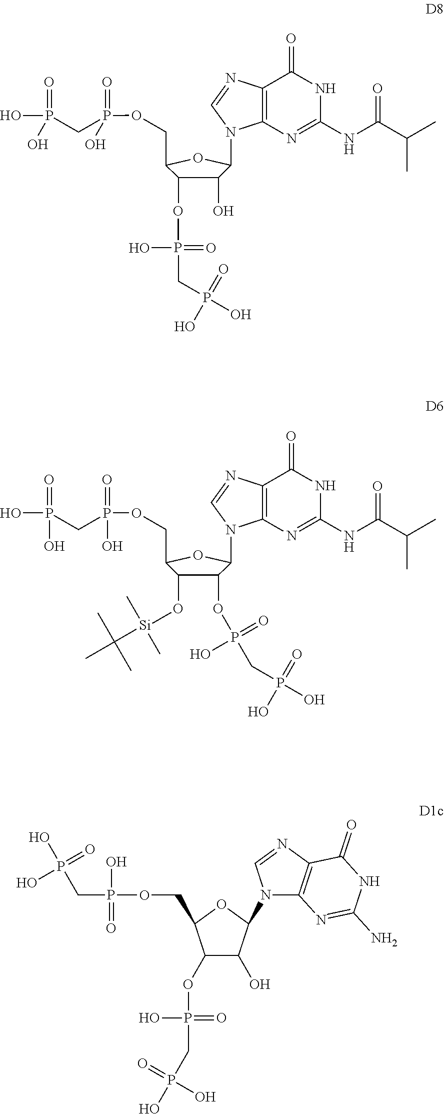

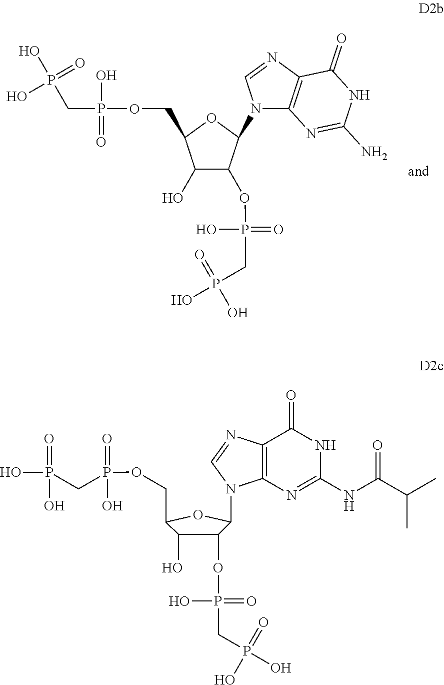

In another aspect, the present invention relates to a method of combating bacteria, or treating bacterial infections, comprising the step of administering to a subject in need thereof a compound of formula (I) or a compound of any of Groups A-E as described herein, or a pharmaceutical composition comprising such compound. In another aspect, the method comprises administering a pharmaceutical composition comprising a compound according to formula (I-a) or a compound of any of Groups A′-E′ as described herein.

In another aspect, the present invention relates to a method of combating bacteria, comprising the step of contacting the bacteria with a compound of formula (I) or a compound of any of Groups A-E as described herein, or a composition comprising such compound. In another aspect, the method comprises administering a composition comprising a compound according to formula (I-a) or a compound of any of Groups A′-E′ as described herein.

In another aspect the present invention relates to the use of a compound of formula (I) or a compound any of Groups A-E as described herein, or a pharmaceutical composition comprising such compound, for the manufacture of a medicament for combating bacteria or treating bacterial infections. In another aspect the pharmaceutical composition comprises a compound of formula (I-a) or a compound according to any of Groups A′-E′ as described herein.

In another aspect, the present invention relates to a compound of formula (I) or a compound of any of Groups A-E as described herein, or to a pharmaceutical composition comprising such compound, or to a compound of formula (I-a) or of Groups A′-E′ as described herein, for use in combating bacteria or treating bacterial infections.

### BRIEF DESCRIPTION OF THE DRAWINGS

FIG. 1: shows the inhibitory effect of EW01 (Compound A1) on RelA synthetic activity in vitro. Results are presented as pmol (p)ppGpp per mg RelA vs. A1 concentration.

FIG. 2: shows the inhibitory effect of EW02 (Compound E3b) on RelA synthetic activity in vitro. Results are presented as pmol (p)ppGpp per mg RelA vs. E3b concentration.

FIG. 3: shows the inhibitory effect of EW03 (Compound D3) on RelA synthetic activity in vitro. Results are presented as pmol (p)ppGpp per mg RelA vs. D3 concentration.

FIG. 4: shows the inhibitory effect of EW04 (Compound D7) on RelA synthetic activity in vitro. Results are presented as pmol (p)ppGpp per mg RelA vs. D7 concentration.

FIG. 5: shows the inhibitory effect of EW05 (Compound D8) on RelA synthetic activity in vitro. Results are presented as pmol (p)ppGpp per mg RelA vs. D8 concentration.

FIG. 6: shows the inhibitory effect of EW07 (Compound D6) on RelA synthetic activity in vitro. Results are presented as pmol (p)ppGpp per mg RelA vs. D6 concentration.

FIG. 7: shows the inhibitory effect of EW03 (Compound D3) on Relseq synthetic activity in vitro. Results are presented as pmol (p)ppGpp per mg Relseq vs. D3 concentration.

FIG. 8: shows the inhibitory effect of Compound A1 on RelA synthetic activity in vitro. Results are presented as % inhibition vs. A1 concentration.

FIG. 9: shows the inhibitory effect of Compound E3b on RelA synthetic activity in vitro. Results are presented as % inhibition vs. E3b concentration.

FIG. 10: shows the inhibitory effect of Compound D3 on RelA synthetic activity in vitro. Results are presented as % inhibition vs. D3 concentration.

FIG. 11: shows the inhibitory effect of Compound D7 on RelA synthetic activity in vitro. Results are presented as % inhibition vs. D7 concentration.

FIG. 12: shows the inhibitory effect of Compound D8 on RelA synthetic activity in vitro. Results are presented as % inhibition vs. D8 concentration.

FIG. 13: shows the inhibitory effect of Compound D6 on RelA synthetic activity in vitro. Results are presented as % inhibition vs. D6 concentration.

FIG. 14: shows the inhibitory effect of Compound D1c on RelA synthetic activity in vitro. Results are presented as % inhibition vs. D1c concentration.

FIG. 15: shows the inhibitory effect of Compound D2b on RelA synthetic activity in vitro. Results are presented as % inhibition vs. D2b concentration.

FIG. 16: shows the inhibitory effect of Compound D2c on RelA synthetic activity in vitro. Results are presented as % inhibition vs. D2c concentration.

FIG. 17: shows the inhibitory effect of Compound D3 on Relseq synthetic activity in vitro. Results are presented as % inhibition vs. D3 concentration.

FIG. 18: shows the inhibitory effect of Compound A1 on Relseq synthetic activity in vitro. Results are presented as % inhibition vs. A1 concentration.

FIG. 19: shows the inhibitory effect of Compound D1c on Relseq synthetic activity in vitro. Results are presented as % inhibition vs. D1c concentration.

FIG. 20: shows the inhibitory effect of Compound D2b on Relseq synthetic activity in vitro. Results are presented as % inhibition vs. D2b concentration.

FIG. 21: shows the inhibitory effect of Compound E3b on Relseq synthetic activity in vitro. Results are presented as % inhibition vs. E3b concentration.

### DETAILED DESCRIPTION OF THE INVENTION

The present invention is based on the discovery of a novel class of compounds which display activity against a wide range of bacteria. The compounds are guanine nucleotide analogs, which inhibit RelA and Relseq synthetic activity and possess anti-bacterial activity. The present invention also relates to pharmaceutical compositions comprising such compounds, and to methods of use thereof for combating bacteria and treating bacterial infections.

In one embodiment, the compounds of the present invention are represented by formula (I), as defined herein.

In an alternative embodiment, the compound may be a compound of Group A (3′(2′) phosphates), for example a compound selected from the group consisting of any of formulae A2, A3 a, A4, A5, A6, A7a, A7b, A7c, A8 and A9, as depicted herein.

In an alternative embodiment, the compound may be a compound of Group B (3′ (2′) amine/azide/amino acid, for example a compound selected from the group consisting of any of formulae B1a, B1b, B1c, B2, B3a, B3b, B3c, B4, B5 and B6, as depicted herein.

In an alternative embodiment, the compound may be a compound of Group C (2′/3′ Sulfamic acid derivatives), for example a compound selected from the group consisting of any of formulae C1, C2, C3, C4, C5 and C6 as depicted herein.

In an alternative embodiment, the compound may be a compound of Group D (ppGpp analogs), for example a compound selected from the group consisting of any of formulae D1, D2, D3, D4, D5, D6, D7 and D8 as depicted herein.

In an alternative embodiment, the compound may be a compound of Group E (2′3′ Cyclic derivatives), for example a compound selected from the group consisting of any of formulae E1, E2, E3a, E4, ES and E6 as depicted herein.

In another aspect, the present invention is based on the finding and realization that the compounds of the invention (i.e., compounds of formula (I) or compounds of Groups A-E), can be active as antibacterial agents. Therefore, in another embodiment, the present invention concerns an pharmaceutical composition comprising a pharmaceutically acceptable carrier or excipient and as an active ingredient a therapeutically effective amount of a compound of formula (I) or a compound any one of Groups A-E, for example compounds of formulae A2, A3a, A4, A5, A6, A7a, A7b, A7c, A8, A9, B1a, B1b, B1c, B2, B3a, B3b, B3c, B4, B5, B6, C1, C2, C3, C4, CS, C6, D1, D2, D3, D4, D5, D6, D7, D8, E1, E2, E3a, E4, E5 and E6 as described hereinabove, or complexes of the aforementioned compounds with negative charge neutralizing agents as described above. Preferably the above compositions are anti-bacterial compositions.

Alternatively, the anti-bacterial pharmaceutical compositions of the present invention comprise a therapeutically effective amount of a compound of formula (I-a), as depicted herein, and a pharmaceutically acceptable carrier or excipient.

In one embodiment, the compound of formula (I-a) may be a compound of Group A′ (3′(2′) phosphate derivatives), such as a compound selected from the group consisting of any of formulae A1, A2, A3, A4, AS, A6, A7, A8 and A9, as depicted herein.

In an alternative embodiment, the compound of formula (I-a) may be a compound of Group B′ (3′(2′) amine/azide/amino acid derivatives), for example a compound selected from the group consisting of any of formulae B1, B2, B3, B4, BS and B6, as depicted herein.

wherein Y is CH₂ or O; X is H or OH; and AA represents an amino acid side chain.

In an alternative embodiment, the compound of formula (I-a) may be a compound of Group C′ (3′(2′) sulfamic acid derivatives), such as a compound selected from the group consisting of any of formulae C1, C2, C3, C4, C5 and C6, as depicted herein.

In an alternative embodiment, the compound of formula (I-a) may be a compound of Group D′ (ppGpp analogs), such as a compound selected from the group consisting of any of formulae D1, D2, D6, D7 and D8, as depicted herein.

In an alternative embodiment, the compound of formula (I-a) may be a compound of Group E′ (cyclic derivatives), such as a compound selected from the group consisting of any of formulae E1, E2, E3, E4, E5 and E6.

Advantageously, the present invention relates to a pharmaceutical composition comprising a pharmaceutically acceptable carrier or excipient, and a therapeutically effective amount of a compound of formula selected from the group consisting of:

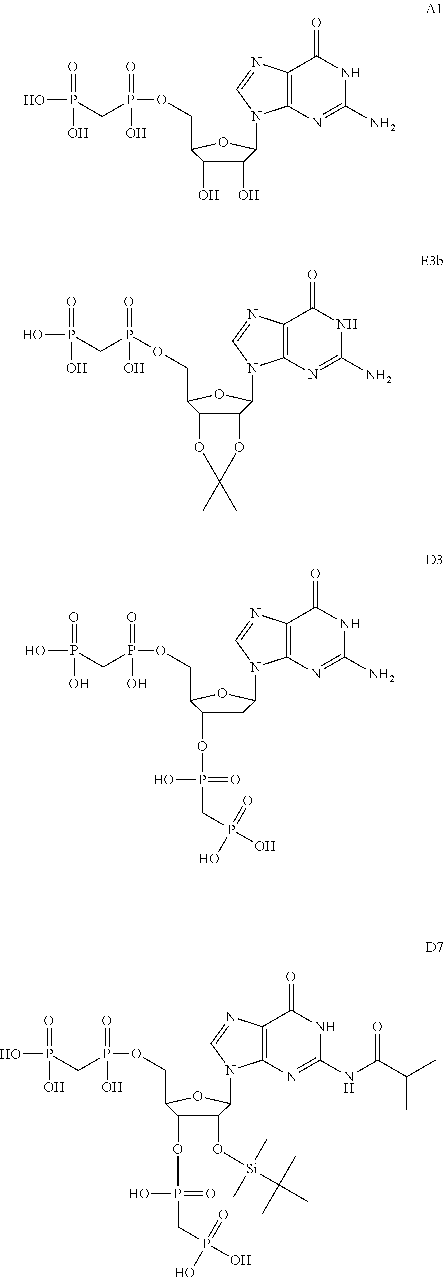

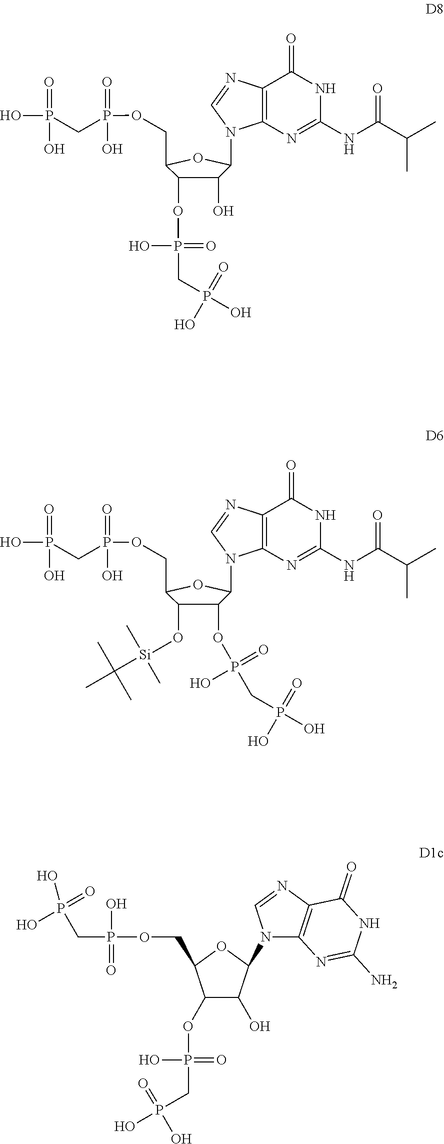

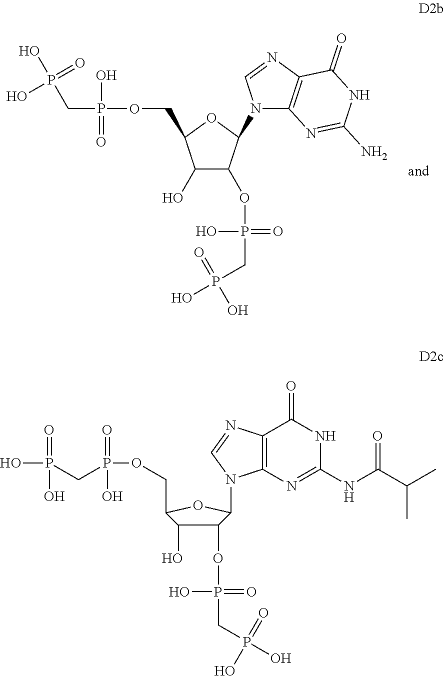

In another aspect, the present invention concerns a pharmaceutical composition comprising as an active ingredient the following compounds or complexes of the following compounds A3 c, D1c, D1b, D2b with negative charge neutralizing agents

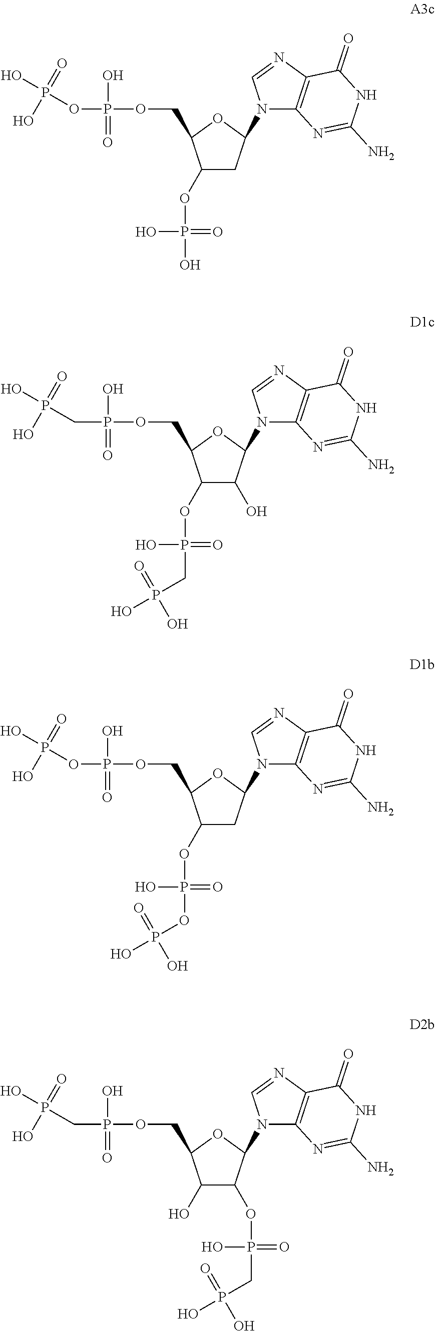

In another aspect, the present invention concerns complexes of compounds formula (I) or (I-a), or compounds of Groups A-E or A′-E′ with “negative charge neutralizing agents”—i.e., agents that when in association with the compounds of formula (I) or (I-a), or compounds of Groups A-E or A′-E′ result in either a neutral or a positively charged complex that can easily penetrate through the bacterial membrane. Non-limiting examples of such agents are polyamines, esterifying agents, phosphoramidating agents, phosphoboronating agents

The term “complex” may refer to electrostatic interaction between the charged compounds of the invention and the opposite charge “negative charge neutralizing agents”. This term may also refer to covalent binding between the charged compounds of the invention and the opposite charge “negative charge neutralizing agents” preferably by bonds that can be cleaved once inside the bacterial cell.

### CHEMICAL DEFINITIONS

The term “C₁ to C₄ alkyl” or “C₁₋₄ alkyl”, used herein alone or as part of another group denotes linear and branched, saturated or unsaturated (e.g., alkenyl, alkynyl) groups, the latter only when the number of carbon atoms in the alkyl chain is greater than or equal to two, and can contain mixed structures. Examples of saturated alkyl groups include but are not limited to methyl, ethyl, n-propyl, isopropyl, n-butyl, iso-butyl, sec-butyl, and tert-butyl. Examples of alkenyl groups include vinyl, allyl, butenyl and the like. Examples of alkynyl groups include ethynyl, propynyl and the like. Similarly, the term “C₁ to C₄ alkylene” or “C₁₋₄ alkylene” denotes a bivalent radical of 1 to 4 carbons.

The C₁ to C₄ alkyl group can be unsubstituted, or substituted with one or more substituents selected from the group consisting of halogen, hydroxy, alkoxy, aryloxy, alkylaryloxy, heteroaryloxy, oxo, cycloalkyl, phenyl, heteroaryl, heterocyclyl, naphthyl, amino, alkylamino, arylamino, heteroarylamino, dialkylamino, diarylamino, alkylarylamino, alkylheteroarylamino, arylheteroarylamino, acyl, acyloxy, nitro, carboxy, carbamoyl, carboxamide, cyano, sulfonyl, sulfonylamino, sulfinyl, sulfinylamino, thiol, C₁ to C₁₀ alkylthio, arylthio, or C₁ to C₁₀ alkylsulfonyl groups. Any substituent can be unsubstituted or further substituted with any one of these aforementioned substituents.

The term “cycloalkyl” generally refers to a C₃ to C₈ cycloalkyl which alone or as part of another group denotes any unsaturated or unsaturated (e.g., cycloalkenyl, cycloalkynyl) monocyclic or polycyclic group. Nonlimiting examples of cycloalkyl groups are cyclopropyl, cyclobutyl, cyclopentyl, cyclohexyl or cycloheptyl. Examples or cycloalkenyl groups include cyclopentenyl, cyclohexenyl and the like. The cycloalkyl group can be unsubstituted or substituted with any one or more of the substituents defined above for alkyl. Similarly, the term “cycloalkylene” means a bivalent cycloalkyl, as defined above, where the cycloalkyl radical is bonded at two positions connecting together two separate additional groups.

The term “aryl” used herein alone or as part of another group denotes an aromatic ring system containing from 6-14 ring carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can be unsubstituted or substituted through available carbon atoms with one or more groups defined hereinabove for alkyl.

The term “heteroaryl” used herein alone or as part of another group denotes a heteroaromatic system containing at least one heteroatom ring atom selected from nitrogen, sulfur and oxygen. The heteroaryl generally contains 5 or more ring atoms. The heteroaryl group can be monocyclic, bicyclic, tricyclic and the like. Also included in this expression are the benzoheterocyclic rings. If nitrogen is a ring atom, the present invention also contemplates the N-oxides of the nitrogen containing heteroaryls. Nonlimiting examples of heteroaryls include thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolyl, quinolyl, naphthyridinyl, quinoxalinyl, quinazolinyl, cinnolinyl, pteridinyl, carbolinyl, thiazolyl, oxazolyl, isothiazolyl, isoxazolyl and the like. The heteroaryl group can optionally be substituted through available atoms with one or more groups defined hereinabove for alkyl. The heteroaryl group can be unsubstituted or substituted through available atoms with one or more groups defined hereinabove for alkyl.

The term “heterocyclic ring” or “heterocyclyl” used herein alone or as part of another group denotes a five-membered to eight-membered rings that have 1 to 4 heteroatoms, such as oxygen, sulfur and/or nitrogen, in particular nitrogen, either alone or in conjunction with sulfur or oxygen ring atoms. These five-membered to eight-membered rings can be saturated, fully unsaturated or partially unsaturated, with fully saturated rings being preferred. Preferred heterocyclic rings include piperidinyl, piperidinyl, pyrrolidinyl pyrrolinyl, pyrazolinyl, pyrazolidinyl, piperidinyl, morpholinyl, thiomorpholinyl, pyranyl, thiopyranyl, piperazinyl, indolinyl, dihydrofuranyl, tetrahydrofuranyl, dihydrothiophenyl, tetrahydrothiophenyl, dihydropyranyl, tetrahydropyranyl, and the like. The heterocyclyl group can be unsubstituted or substituted through available atoms with one or more groups defined hereinabove for alkyl.

The term “hydroxy protecting group” as used herein refers to a readily cleavable group bonded to a hydroxyl (i.e., OH) group. The nature of the hydroxy-protecting group is not critical so long as the derivatized hydroxyl group is stable. Suitable examples of a hydroxy protecting group include a silyl group, which can be substituted with alkyl (trialkylsilyl), with an aryl (triarylsilyl) or a combination thereof (e.g., dialkylphenylsilyl). A preferred example of a silyl protecting group is trimethylsilyl (TMS) or t-butyldimethyl silyl (TBDMS). Other examples of hydroxy protecting groups include, for example, C₁-C₄ alkyl, —CO—(C₁-C₆ alkyl), —SO₂—(C₁-C₆ alkyl), —SO₂-aryl, —CO—Ar in which Ar is an aryl group as defined above, and —CO—(C₁-C₆ alkyl)Ar (e.g., a carboxybenzyl group). Other examples of hydroxy-protecting groups are described by C. B. Reese and E. Haslam, “Protective Groups in Organic Chemistry,” J. G. W. McOmie, Ed., Plenum Press, New York, N.Y., 1973, Chapters 3 and 4, respectively, and T. W. Greene and P. G. M. Wuts, “Protective Groups in Organic Synthesis,” 2nd ed., John Wiley and Sons, New York, N.Y., 1991, Chapters 2 and 3, each of which is incorporated herein by reference.

The term “amino protecting group” as used herein refers to a readily cleavable group bonded to an amino group. The nature of the amino protecting group is not critical so long as the derivatized amino group is stable. Exemplary amino-protecting groups include t-butoxycarbonyl, benzyloxycarbonyl, acetyl, phenylcarbonyl, or a silyl group, which can be substituted with alkyl (trialkylsilyl), with an aryl (triarylsilyl) or a combination thereof (e.g., dialkylphenylsilyl), e.g., trimethylsilyl (TMS) or t-butyldimethyl silyl (TBDMS). Other suitable amino-protecting agents and amino-protecting groups, as well as methods of protection and deprotection, have been described in, e.g., T. W. Greene and P. G. M. Wuts, Protective Groups in Organic Synthesis, 2^{nd} Ed., John Wiley and Sons (1991) and A. J. Pearson and W. R. Roush, Activating Agents and Protecting Groups, John Wiley and Sons (1999), each of which is incorporated herein by reference.

Several of the compounds of the present invention contain side chains of amino acids. The invention encompasses compounds having side chains of natural and unnatural amino acids, meaning both the naturally occurring amino acids and other unnaturally amino acids including both optically active (D and L) forms as well as racemic derivatives. The naturally occurring amino acids are, e.g., glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine and lysine. Examples of unnatural α-amino acids include, but are not limited to, α-aminoisobutyric acid, α-aminobutyric acid, γ-aminobutyric acid, citrulline, homocitrulline, homoproline, homoserine, hydroxyproline, norleucine, 4-aminophenylalanine, 4-halo phenyl alanine (e.g., 4-fluoro, bromo, chloro or iodo phenylalanine wherein), 4-nitro phenylalanine, statine, hydroxylysine, kynurenine, 3-(2′-naphthyl)alanine, 3-(1′-naphthyl)alanine, methionine sulfone, (t-butyl)alanine, (t-butyl)glycine, 4-hydroxyphenylglycine, aminoalanine, phenylglycine, vinylalanine, propargyl-gylcine, 1,2,4-triazolo-3-alanine, thyronine, 6-hydroxytryptophan, 5-hydroxytryptophan, 3-hydroxykynurenine, 3-aminotyrosine, trifluoromethyl-alanine, 2-thienylalanine, (2-(4-pyridyl)ethyl)cysteine, 3,4-dimethoxy-phenylalanine, 3-(2′-thiazolyl)alanine, ibotenic acid, 1-amino-1-cyclopentane-carboxylic acid, 1-amino-1-cyclohexanecarboxylic acid, quisqualic acid, 3-(trifluoromethylphenyl)alanine, (cyclohexyl)glycine, thiohistidine, 3-methoxytyrosine, elastatinal, norleucine, norvaline, alloisoleucine, homoarginine, thioproline, dehydroproline, hydroxyproline, homoproline, α-amino-n-butyric acid, cyclohexylalanine, 2-amino-3-phenylbutyric acid, β-2- and 3-thienylalanine, β-2- and 3-furanylalanine, β-2-, 3- and 4-pyridylalanine, β-(benzothienyl-2- and 3-yl)alanine, β-(1- and 2-naphthyl)alanine, O-alkylated derivatives of serine, threonine or tyrosine, S-alkylated cysteine, S-alkylated homocysteine, O-sulfate, O-phosphate and O-carboxylate esters of tyrosine, 3-(sulfo)tyrosine, 3-(carboxy)tyrosine, 3-(phospho)tyrosine, the 4-methane sulfonic acid ester of tyrosine, 4-methane phosphonic acid ester of tyrosine, 3,5-diiodotyrosine, 3-nitrotyrosine, ε-alkyl lysine, and δ-alkyl ornithine, Dap (i.e., the side chain is CH₂NH₂), dimethyl Dap (i.e., the side chain is CH₂N(CH₃)₂), dimethylamino lysine (i.e., the side chain is (CH₂)₄—N(CH₃)₂), Dab (i.e., the side chain is CH₂CH₂NH₂), Abu (i.e., the side chain is CH₂CH₃), Apn (i.e., the side chain is CH₂CH₂CH₃), and Ahx (i.e., the side chain is (CH₂)₃—CH₃).

All stereoisomers, optical and geometrical isomers of the compounds of the instant invention are contemplated, either in admixture or in pure or substantially pure form. The compounds of the present invention can have asymmetric centers at any of the atoms. Consequently, the compounds can exist in enantiomeric or diastereomeric forms or in mixtures thereof. The present invention contemplates the use of any racemates (i.e. mixtures containing equal amounts of each enantiomers), enantiomerically enriched mixtures (i.e., mixtures enriched for one enantiomer), pure enantiomers or diastereomers, or any mixtures thereof. The chiral centers can be designated as R or S or R,S or d,D, l,L or d,l, D,L. Compounds comprising amino acid residues include residues of D-amino acids, L-amino acids, or racemic derivatives of amino acids. In addition, several of the compounds of the invention contain one or more double bonds. The present invention intends to encompass all structural and geometrical isomers including cis, trans, E and Z isomers.

One or more of the compounds of the invention, may be present as a salt. The term “salt” encompasses both basic and acid addition salts, including but not limited to phosphate, dihydrogen phosphate, hydrogen phosphate and phosphonate salts, and include salts formed with organic and inorganic anions and cations. Furthermore, the term includes salts that form by standard acid-base reactions of basic groups and organic or inorganic acids. Such acids include hydrochloric, hydrofluoric, hydrobromic, trifluoroacetic, sulfuric, phosphoric, acetic, succinic, citric, lactic, maleic, fumaric, cholic, pamoic, mucic, D-camphoric, phthalic, tartaric, salicyclic, methanesulfonic, benzenesulfonic, p-toluenesulfonic, sorbic, picric, benzoic, cinnamic, and like acids.

The term “organic or inorganic cation” refers to counter-ions for an acid, for example the counter-ions for phosphates or phosphonates. The counter-ions can be chosen from the alkali and alkaline earth metals, (such as lithium, sodium, potassium, barium, aluminum and calcium); ammonium and mono-, di- and tri-alkyl amines such as trimethylamine, cyclohexylamine; and the organic cations, such as dibenzylammonium, benzylammonium, 2-hydroxyethylammonium, bis(2-hydroxyethyl)ammonium, phenylethylbenzylammonium, dibenzylethylene diammonium, and like cations. See, for example, “Pharmaceutical Salts,” Berge et al., J. Pharm. Sci., 66:1-19 (1977), which is incorporated herein by reference. Furthermore, any zwitterionic form of the instant compounds formed by a carboxylic acid and an amino group are also contemplated.

The present invention also includes solvates of the compounds of the present invention and salts thereof. “Solvate” means a physical association of a compound of the invention with one or more solvent molecules. This physical association involves varying degrees of ionic and covalent bonding, including hydrogen bonding. In certain instances the solvate will be capable of isolation. “Solvate” encompasses both solution-phase and isolatable solvates. Non-limiting examples of suitable solvates include ethanolates, methanolates and the like. “Hydrate” is a solvate wherein the solvent molecule is water.

The present invention also includes polymorphs of the compounds of the present invention and salts thereof. The term “polymorph” refers to a particular crystalline state of a substance, which can be characterized by particular physical properties such as X-ray diffraction, IR spectra, melting point, and the like.

### Therapeutic Uses:

As contemplated herein, the present invention is based on the finding that compounds of formula (I), (I-a) or compounds of groups A-E or A′-E′ as described above are active as antibacterial agents. It is further contemplated that the compounds of the present invention act by inhibiting the synthetic pathways of RelA and/or Relseq. It is apparent to a person of skill in the art that the purported mechanism by which the compounds of the present invention act does not limit the broad scope of the invention.

The antibacterial compositions of the invention may be used for medicinal purposes and in such a case the composition is a pharmaceutical composition for the treatment of bacterial infections.

Thus, another aspect, the present invention relates to a method of combating bacteria, or treating bacterial infections, comprising the step of administering to a subject in need thereof a compound of formula (I) or a compound of any of Groups A-E as described herein, or a pharmaceutical composition comprising such compound. In another aspect, the method comprises administering a pharmaceutical composition comprising a compound according to formula (I-a) or a compound of any of Groups A′-E′ as described herein. In another embodiment, the method comprises administering a pharmaceutical composition comprising an effective amount of any a complex of a compound of the present invention with a negative charge neutralizing agent.

In another aspect the present invention relates to the use of a compound of formula (I) or a compound any of Groups A-E as described herein, or a pharmaceutical composition comprising such compound, for the manufacture of a medicament for combating bacteria or treating bacterial infections. In another aspect the pharmaceutical composition comprises a compound of formula (I-a) or a compound according to any of Groups A′-E′ as described herein, or a complex of such compound with a negative charge neutralizing agent.

In another aspect, the present invention relates to a compound of formula (I) or a compound of any of Groups A-E as described herein, or to a pharmaceutical composition comprising such compound, or to a compound of formula (I-a) or of Groups A′-E′ as described herein, or a complex of such compound with a negative charge neutralizing agent for use in combating bacteria or treating bacterial infections.

The anti bacterial composition may also be used for disinfecting purposes for example of surfaces, devices (including medical devices), cultures of eukaryotic cells or tissue, water pipes and water filters, food and agricultural products. The present invention further concerns a method for combating bacteria the method comprising contacting the bacteria with an effective amount of compound of formula (I) or a compound of any of Groups A-E as described herein, or with a complex of these compounds with a negative charge neutralizing agent, or a pharmaceutical composition comprising such compound. In another aspect, the method comprises administering a pharmaceutical composition comprising a compound according to formula (I-a) or a compound of any of Groups A′-E′ as described herein, or with a complex of these compounds with a negative charge neutralizing agent. The contact may be ex vivo on a surface, on a device, in cell/tissue culture dish, in food, water, agricultural product as described above. Alternatively the contact may be in the body of a human or non human subject.

The term “anti-bacterial” may refer to one or more of the following effects: killing the bacteria (bacteriocide), causing halt of growth of bacteria (bacteriostatic), prevention of bacterial infection, prevention of bio-film formation and disintegration of a formed biofilm, and decrease in bacterial virulence.

Examples of bacterial strain that can be treated/disinfected by the composition of the invention (both as a disinfecting composition and as a pharmaceutical composition) are all gram negative and gram positive bacteria and in particular pathogenic gram negative and gram positive bacteria.

The term “combating bacteria” or “treating bacterial infection” may refer to one of the following: decrease in the number of bacteria, killing or eliminating the bacteria, inhibition of bacterial growth (stasis), inhibition of bacterial infestation, inhibition of biofilm formation, disintegration of existing biofilm, or decrease in bacterial virulence.

The methods of the invention both ex-vivo and in the body of the subject may further comprise co administration of at least one additional anti-bacterial agent such as state of the art antibiotics.

### Pharmaceutical Compositions

The pharmaceutical compositions of the present invention can be formulated for administration by a variety of routes including oral, vaginal, rectal, ocular, transdermal, parenteral (subcutaneous, intraperitoneal, intravenous, intraarterial, transdermal and intramuscular), mucosal, topical, intranasal, via a suppository or by inhalation. Such compositions are prepared in a manner well known in the pharmaceutical art and comprise as an active ingredient at least one compound of the present invention as described herein, and a pharmaceutically acceptable excipient or a carrier. The term “pharmaceutically acceptable” means approved by a regulatory agency of the Federal or a state government or listed in the U.S. Pharmacopeia or other generally recognized pharmacopeia for use in animals and, more particularly, in humans.

During the preparation of the pharmaceutical compositions according to the present invention the active ingredient is usually mixed with a carrier or excipient, which may be a solid, semi-solid, or liquid material. The compositions can be in the fond of tablets, pills, capsules, pellets, granules, powders, lozenges, sachets, cachets, elixirs, suspensions, dispersions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In another embodiment, the compounds of the present invention can be added to a person's diet by mixing them with food or drink.

The carriers may be any of those conventionally used and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

The amount of a compound of the invention that will be effective in the treatment of a particular anti-bacterial infection, will depend on the nature of the infection, and can be determined by standard clinical techniques. In addition, in vitro assays may optionally be employed to help identify optimal dosage ranges. The precise dose to be employed in the formulation will also depend on the route of administration, and the seriousness of the condition, and should be decided according to the judgment of the practitioner and each patient's circumstances. A preferred dosage will be within the range of 0.01-1000 mg/kg of body weight, more preferably, 0.1 mg/kg to 100 mg/kg and even more preferably 1 mg/kg to 10 mg/kg. Effective doses may be extrapolated from dose-response curves derived from in vitro or animal model test bioassays or systems.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLE 1

### Synthesis Procedures

### Group A—3′(2′) (Phosphate)

### Preparation of A4 where Y═CH₂ and X═H

### Step 1—²N-Isobutyryl-3′-O-(2-Cyanoethyl)H-Phosphonate-5′O-Dimetoxytrityl deoxyguanosine (A4-I)

²N-Isobutyryl-50-Dimetoxytrityl deoxyguanosine (0.5 g, 0.78 mmol) was dried by co-evaporation with dry toluene and suspended in dry pyridine (10 mL) under inert atmosphere. Diphenyl phosphite (250 μL, 1.3 mmol) was added and stirred for 2 h. 3-hydroxypropionitrile (150 μL, 2.16 mmol) was added. After stirring for 2 hr, the solvent was evaporated. The oily crude was used without further purification.

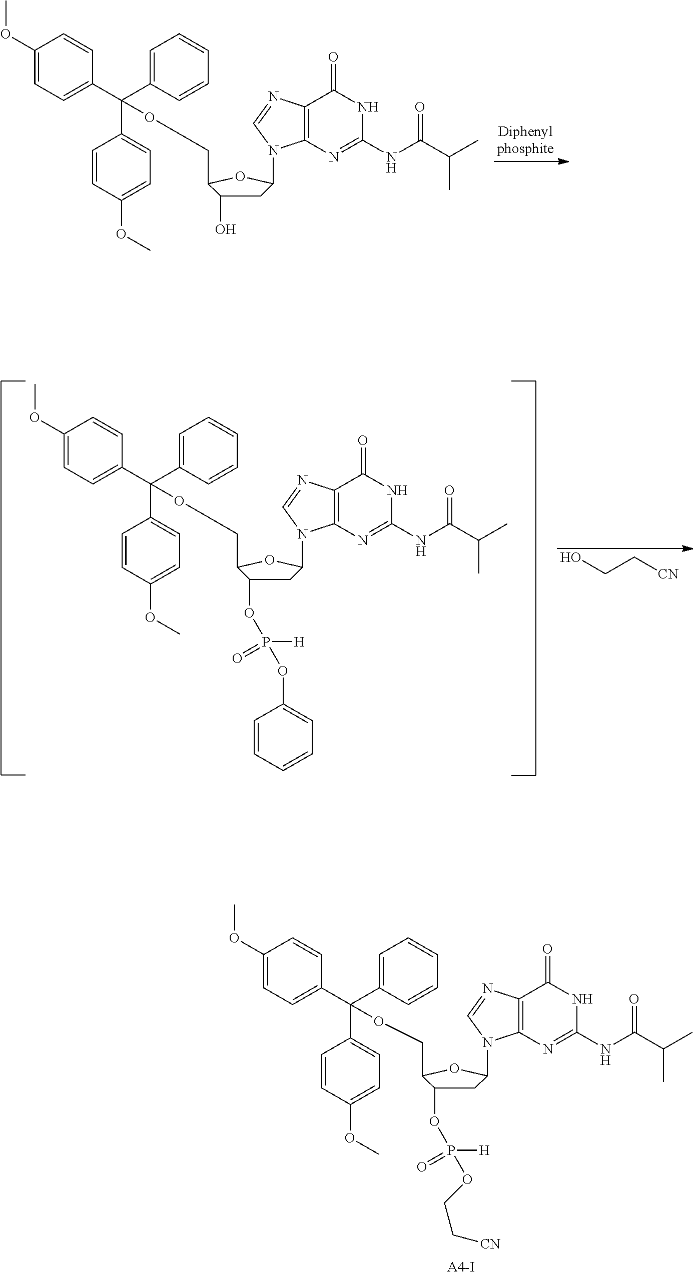

### Step 2—²N-Isobutyryl-3′-O-(2-Cyanoethyl)H-Phosphonate-deoxyguanosine (A4-II)

400 mg of A4-I were dissolved in 20 mL of 3% TCA in DCM and stirred for 20 minutes. The solvent was evaporated and the crude partitioned between DCM and aq. NH₄HCO₃ (20 mL each). The organic phase was washed twice with ammonium bicarbonate and twice with water. Then it was dried over anhydrous Na₂SO₄, filtered and concentrated under vacuum. The oily crude was used without further purification.

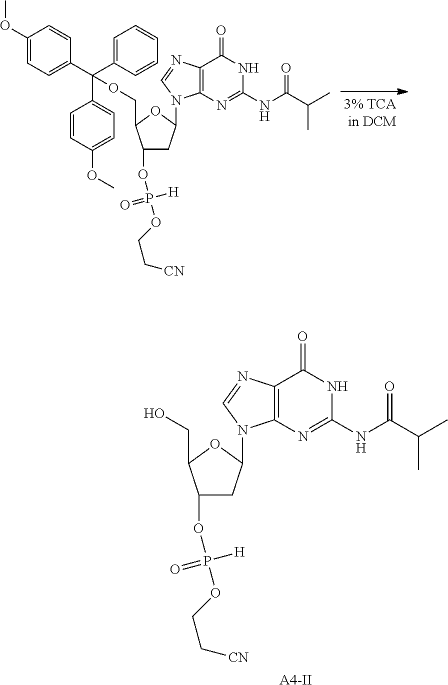

### Step 3—²N-Isobutyryl-5′-O-methylene-(bisphosphonate)-3′-O-(2-Cyanoethyl)H-Phosphonate-2′ deoxyguanosine (A4)

A solution of methylenebis (phosphonic dichloride) (240 mg, 0.96 mmol) in trimethyl phosphate (10 mL) cooled to 0° C. was added to a suspension of A4-II (250 mg, 0.55 mmol) in trimethyl phosphate (10 mL) at 0° C. The reaction mixture was stirred at 0° C. After 1 h, 0.7 M aqueous ammonium bicarbonate (pH 7.0) was added. Chromatographic purification on HPLC DEAE-Sephadex, using a 0-1 M gradient of ammonium bicarbonate, gave a glassy solid (180 mg, 75%).

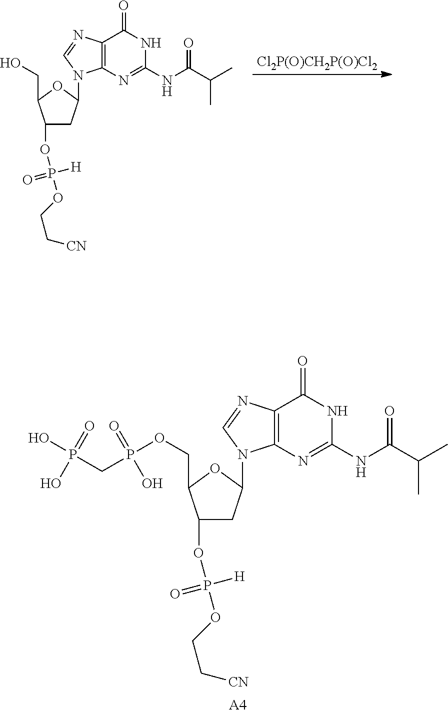

### Preparation of A5 where Y═CH₂; X═H and R═O(CH₂)₃N(CH₃)₂

### 5′-O-methylene-(bisphosphonate)-3′-O-(N,N-dimethyl-3-aminopropyl)Phosphate-T-deoxyguanosine

A4 was dissolved in 10 mL of a solution of CCl₄ in pyridine (50%) containing 10% (v/v) of N,N-dimethyl-3-aminopropanol. The mixture was stirred for 30 minutes and the solvents were evaporated. The presence of the desired compound was detected by ESI-MS (m/z: calc. 713.5, found: 714.9). The crude was subjected to hydrolysis overnight at 60° C. in 25% ammonium hydroxide. After lyophilization, the crude product was applied to a SAX HPLC semipreparative column. The desired product was obtained as a white powder.

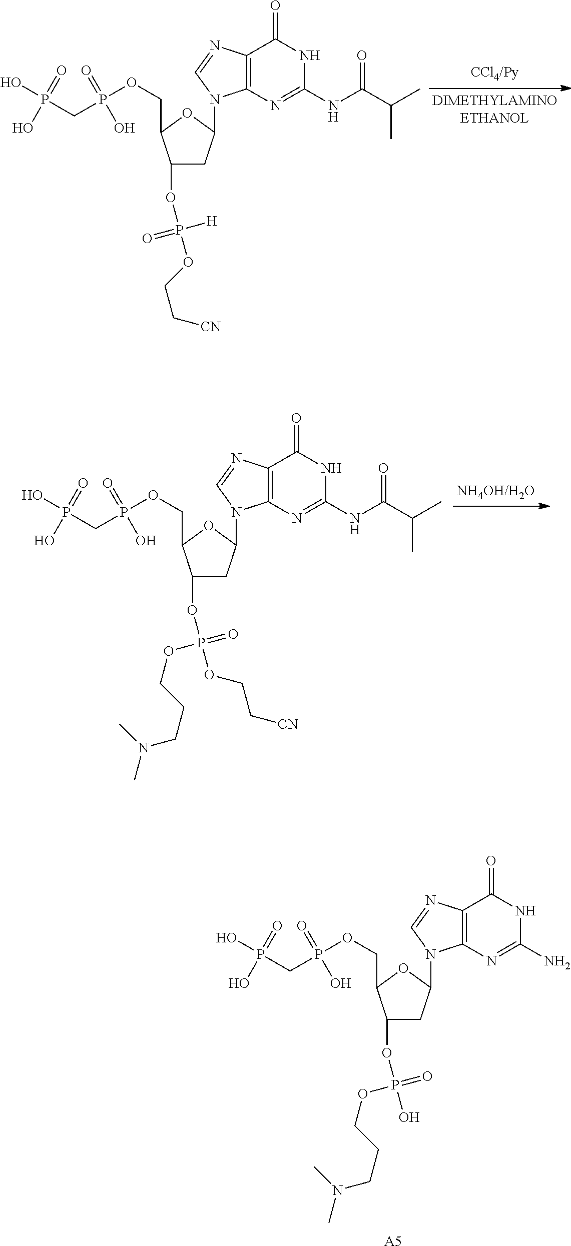

### Preparation of A2 where Y═CH₂ and X═H

### 5′-O-methylene-(bisphosphonate)-3′-O—(H-Phosphonate)-2′-deoxyguanosine

Compound A4 was subjected to hydrolysis overnight at 60° C. in 25% ammonium hydroxide. After lyophilized, the crude was applied to a SAX HPLC semipreparative column. The desired product was obtained as a white powder.

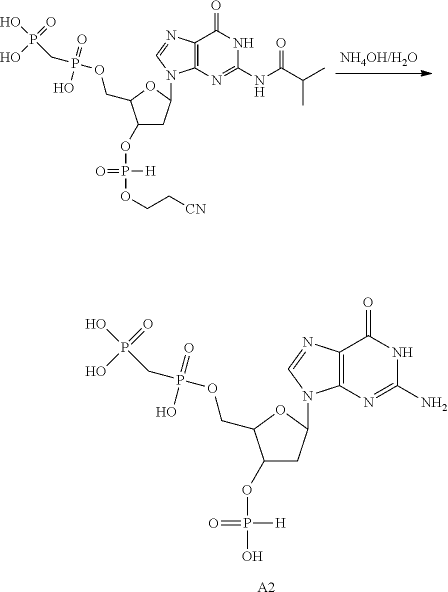

### Preparation of A3 where Y═CH₂ and X═H

### 5′-O-methylene-(bisphosphonate)-3′-(Phosphate)-2′-deoxyguanosine

Compound A2 was oxidized with a mixture of CCl₄/Pyridine/water (5/5/1) for 30 minutes. The solvents were then evaporated and the crude applied to a SAX HPLC semipreparative column. The desired product was obtained as a white powder.

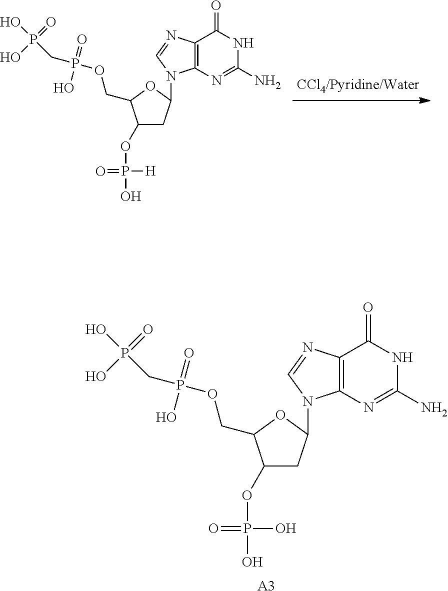

### Preparation of A9 where Y═CH₂, X═OH

### Step 1—N²-Isobutyrylguanosine (A9-I)

Guanosine hydrate (10 g, 35.3 mmol) was dried by co-evaporation of its suspension in dry pyridine (3×100 mL) in vacuum. The residue was suspended in dry pyridine (250 mL) under a nitrogen atmosphere, and chlorotrimethylsilane (28.8 g, 265 mmol) was added. The reaction mixture was stirred at ambient temperature for 2 h, cooled to 0° C., and isobutyryl chloride (11.3 g, 106 mmol) was added dropwise over 20 min. The mixture was allowed to warm to room temperature and stirred for 3 h. The reaction mixture was cooled to 0° C., and the reaction was quenched by addition of H₂O (30 mL). After stirring for 5 min at 0° C. and then 5 min at room temperature, concentrated aqueous NH₄OH (65 mL) was added. After stirring for an additional 15 min at room temperature, the mixture was diluted with H₂O (500 mL) and washed with CH₂Cl₂ (200 mL). The aqueous layer was concentrated by evaporation in vacuum. The residue was recrystallized from hot H₂O to obtain N²-isobutyrylguanosine (9.90 g, 79%) as a white solid.

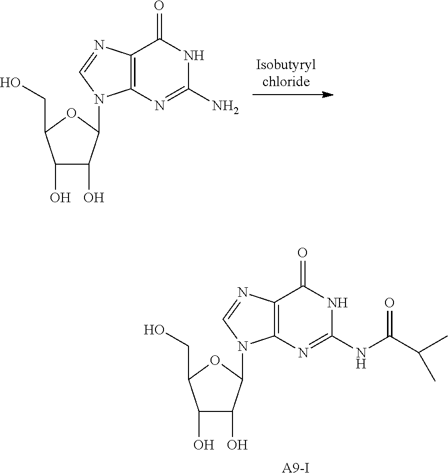

Step 2—²N-Isobutyryl-5′O-Dimetoxytrityl Guanosine (A9-II)

²N-Isobutyryl guanosine was dried by co-evaporation with dry pyridine three times. To a stirred suspension of dry ²N-Isobutyryl guanosine (5 g; 14.15 mmol) in pyridine (100 mL), a solution of dimetoxytrityl chloride (5 g; 14.8 mmol) in pyridine (30 mL) was added dropwise over a period of 60 min. The reaction mixture was left for 4 h at room temperature, cooled to 0° C. by immersion in an ice water bath, quenched with 5% NaHCO₃ (100 mL), and extracted with ethyl acetate (3×100 mL). The organic fractions were pooled, dried over magnesium sulfate, concentrated in vacuum and the residue was co-evaporated with toluene. The gum oil residue was dissolved in a minimum amount of methylene chloride and added dropwise to a mixture of ethyl ether and petroleum ether (2000 mL 75/25) with stirring. After 20 min, pure ²N-Isobutyryl-5′O-Dimetoxytrityl Guanosine precipitated from the solution and was filtered off and dried.

Yield: 75%

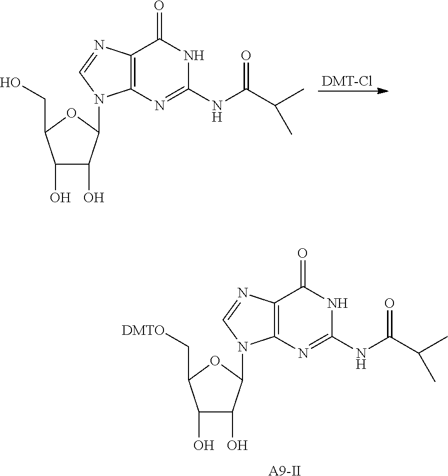

### Step 3—²N-Isobutyryl-5′O-Dimetoxytrityl-2′O-tertbutyldimethylsilyl Guanosine (A9-III) and ²N-Isobutyryl-5′O-Dimetoxytrityl-3′O-tertbutyldimethylsilyl Guanosine (A9-IV)

Compound (A9-II) was dried by co-evaporation with toluene three times. To a stirred solution of (A9-II) (4 g; 6.1 mmol) in 100 mL of dry CH₂Cl₂ under nitrogen atmosphere, imidazol (1.25 g; 18.3 mmol) and tertbutyldimethylsilyl chloride (2.75 g; 18.3 mmol) were added. The reaction mixture was stirred overnight and quenched with 5% NaHCO₃ (100 mL). The organic layer was concentrated in vacuum and the resulting residue co-evaporated with toluene. The obtained isomers (2′O TBDMS and 3′O TBDMS) were separated using preparative reversed phase HPLC.

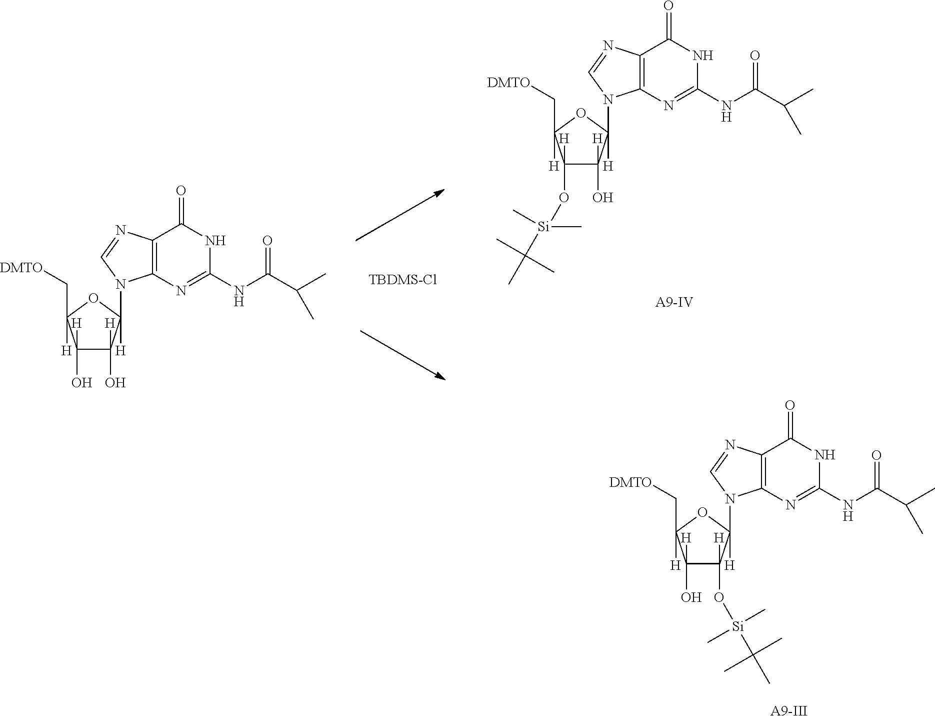

### Step 4—²N-Isobutyryl-5′O-Dimetoxytrityl-3′O-tertbutyldimethylsilyl-2′-O-(2-cyanoethyl)H-Phosphonate Guanosine (A9-V)

Compound (A9-IV) (0.78 mmol) was dried by co-evaporation with dry toluene and suspended in dry pyridine (10 mL) under inert atmosphere. Diphenyl phosphite (250 μL, 1.3 mmol) was added and stirred for 2 h. 3-hydroxypropionitrile (150 μL, 2.16 mmol) was added. After stirring for 2 hr, the solvent was evaporated. The oily crude was used without further purification.

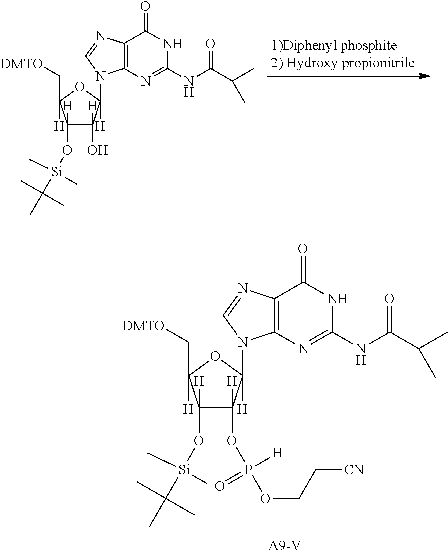

### Step 5—²N-Isobutyryl-3′O-tertbutyldimethylsilyl-2′-O-(2-cyanoethyl)-H-Phosphonate-guanosine (A9-VI)

Same procedure as used for the preparation of compound (A4-II), using compound (A9-V) as starting material.

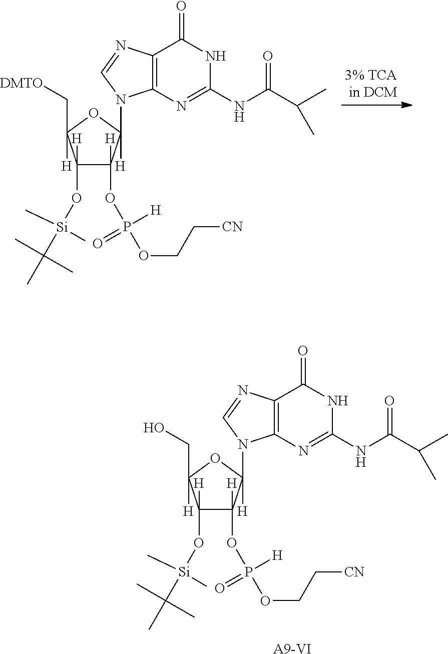

### Step 6—²N-Isobutyryl-5′-O-methylene-(bisphosphonate)-2′-O-(2-cyanoethyl)-H-Phosphonate-guanosine (A9)

Same procedure as used in the last step of the preparation of compound (A4), using compound (A8-VI) as starting material.

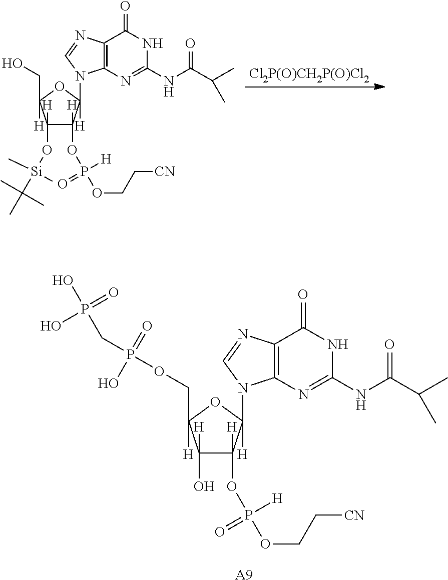

### Preparation of A8 where Y═CH₂, X═OH; R═R₃

Same procedure as used for the preparation of compound number (A5), using compound number (A9) as starting material and Fmoc-protected amino alcohols bearing amino acid's side chains.

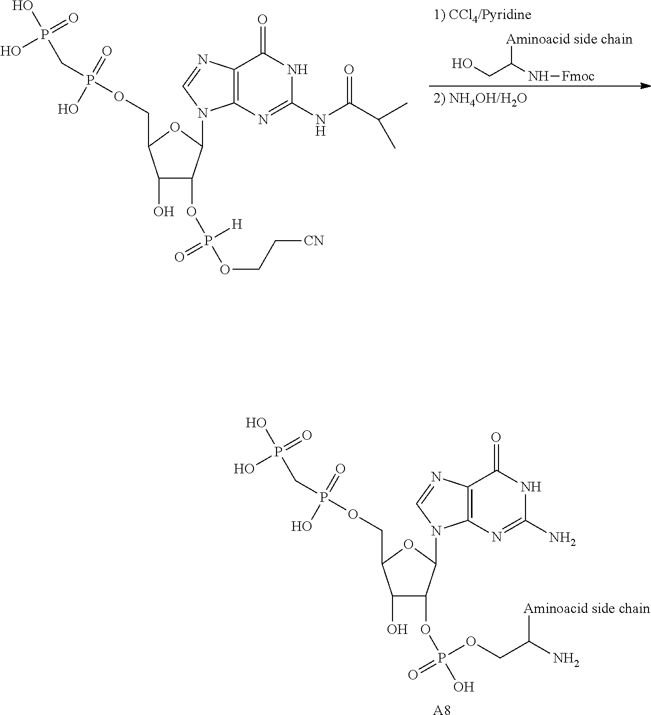

### Preparation of A6 where Y═CH₂ and X═OH

Same procedure as used for the preparation of compound A2, using compound (A9) as starting material.

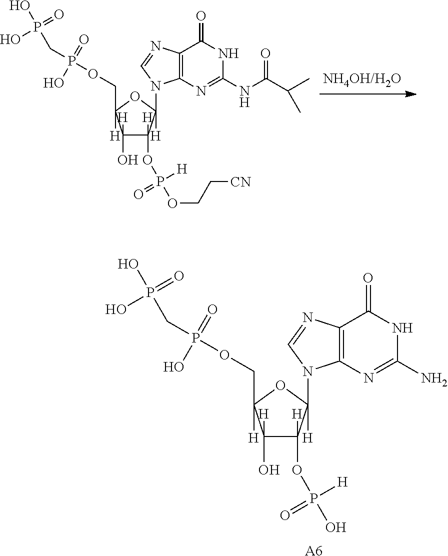

### Group B: 3′ (2′) Amine/Azide/Amino Acid

### Preparation of B5 where Y═CH₂ and X═H

### Step 1—N²-Isobutyryl-2′-deoxyguanosine (B5-I)

2′-Deoxyguanosine (5.705 g, 20 mmol) was co-evaporated with anhydrous pyridine and then suspended in 200 mL anhydrous pyridine. Trimethylchlorosilane (13 mL, 100 mmol) was added slowly to the suspension cooled in an ice-bath. After 30 min, isobutyric anhydride (17 mL, 100 mmol) was added dropwise and the reaction mixture was stirred for 2.5 h at room temperature. The reaction mixture was then chilled in an ice-bath, 40 mL of cold water was added and let stir for 15 min. Concentrated aqueous NH₄OH was added, let stir for another 30 min and rotovapped to give oil with salts. Water was added until all salts dissolved and washed once with equal volume of ether. The product crystallizes immediately in the aqueous layer, which was filtered and dried on vacuum until constant weight to give 5.575 g of 1 (82% yield) as a white solid.

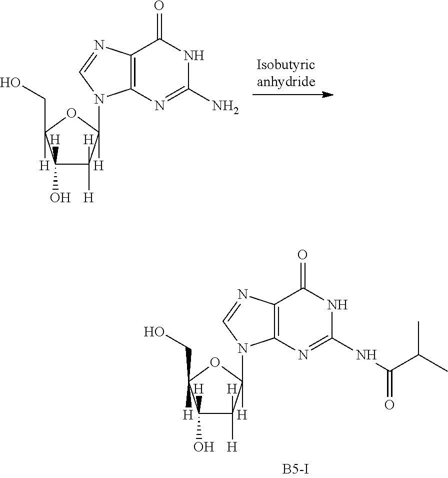

### Step 2—²N-Isobutyryl-5′O-Dimetoxytrityl deoxyguanosine (B5-II)

²N-Isobutyryl deoxyguanosine (B5-I) was dried by co-evaporation with dry pyridine three times. To a stirred suspension of dry (B5-I) (14.15 mmol) in pyridine (100 mL), a solution of dimetoxytrityl chloride (14.8 mmol) in pyridine (30 mL) was added dropwise over a period of 60 min. The reaction mixture was left for 4 h at room temperature, cooled to 0° C. by immersion in an ice water bath, quenched with 5% NaHCO₃ (100 mL), and extracted with ethyl acetate (3×100 mL). The organic fractions were pooled, dried over magnesium sulfate, concentrated in vacuum and the residue was co-evaporated with toluene. The gum oil residue was dissolved in a minimum amount of methylenechloride and added dropwise to a mixture of ethyl ether and petroleum ether (2000 mL 75/25) with stirring. After 20 min, pure ²N-Isobutyryl-5′O-Dimetoxytrityl deoxyguanosine precipitated from the solution and was filtered off and dried.

Yield: 75%

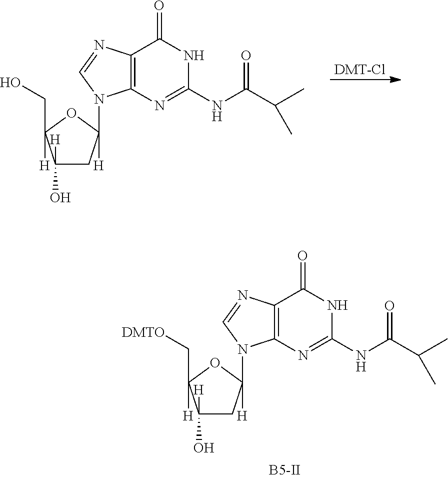

### Step 3—3′-O(R)-(methylsulfonyl)²N-Isobutyryl-5′O-Dimetoxytrityl deoxyguanosine (B5-III)

Methanesulfonyl chloride (0.12 mol) was added dropwise over a period of 15 min to a cooled (ice-water-bath) solution of (B5-II) (0.06 mol) in dry pyridine (80 mL). The cooled reactants were then stirred at ˜0° C. for 15 h. The products were then poured into a vigorously stirred ice water mixture (1500 g). The precipitated solid was collected by filtration, washed with water (4×100 mL) and dried in vacuo over P₂O₅

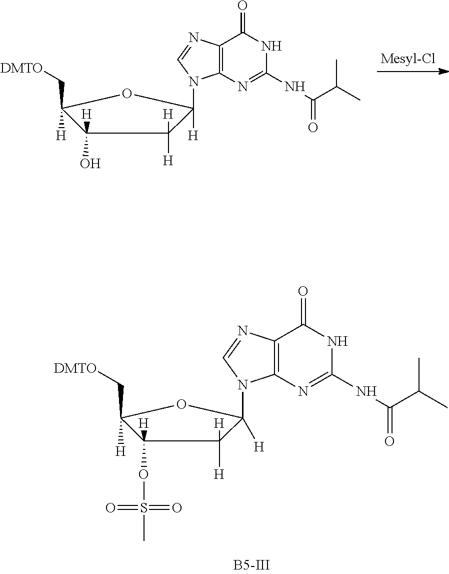

### Step 4—3′-O(S)-(Benzoate)²N-Isobutyryl-5′O-Dimetoxytrityl deoxyguanosine (B5-IV)

A mixture of compound (B5-III) (100 mmol) and sodium benzoate (200 mmol) in dimethylsulfoxide (320 mL) was stirred at 90° C. overnight and cooled to room temperature. The mixture was diluted with ethyl acetate (600 mL) and washed successively with water and brine. The organic phase was dried over magnesium sulfate and the solvent was evaporated in vacuo to give an oil. The oil was crystallized from n-hexane to give 31.0 g (88.7%)

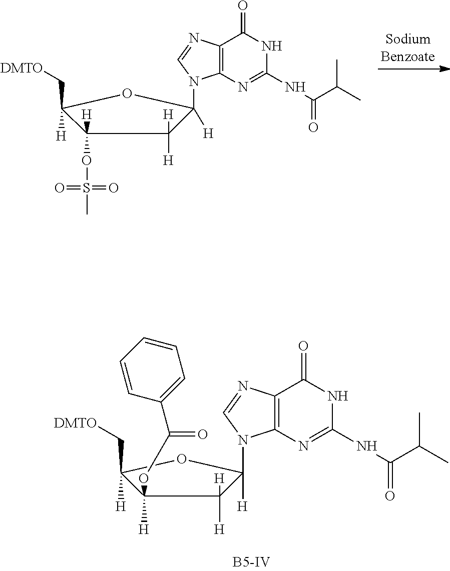

### Step 5—3′-OH(S)-²N-Isobutyryl-5′O-Dimetoxytrityl deoxyguanosine (B5-V)

To a solution of (B5-IV) (30 g, 85.9 mmol) in methanol (600 mL) was added potassium carbonate (11.9 g, 86.1 mmol) and the mixture was stirred at room temperature for one hour. The solution was diluted with ethyl acetate (1 L) and washed with water. The organic phase was washed with brine. The aqueous phase was saturated with sodium chloride, extracted with chloroform and washed with brine. The combined organic extracts were dried over magnesium sulfate and evaporated in vacuo to give an oil. The oil was chromatographed on a silica gel (500 g) column with a mixture of n-hexane and ethyl acetate (1:1) as an eluent to give 20.7 g (97%) of (B5-V) as colorless crystals

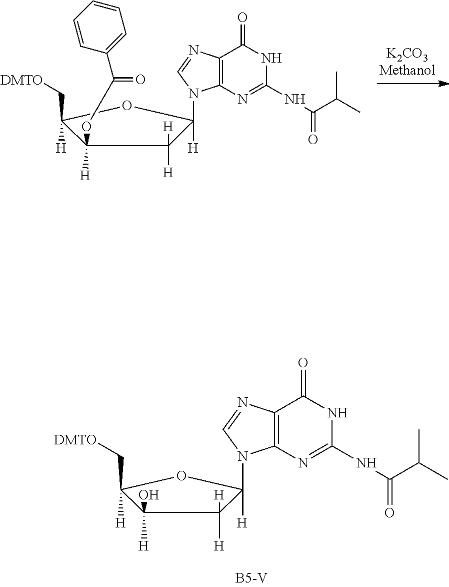

### Step 6—3′-O(S)-(methylsulfonyl)²N-Isobutyryl-5′O-Dimetoxytrityl deoxyguanosine (B5-VI)

The procedure is as described above for compound (B5-III) using compound (B5-V) as starting material.

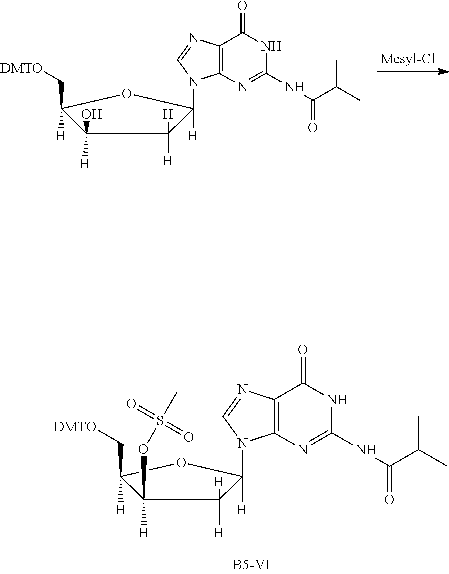

### Step 7—3′-(R)-(azido)²N-Isobutyryl-5′O-Dimetoxytrityl deoxyguanosine (B5-VII)

A mixture of (B5-VI) (81.5 mmol) and sodium azide (163 mmol) in dimethyl sulfoxide (350 mL) was stirred at 90° C. overnight and the solution was diluted with ethyl acetate (600 mL). The solution was washed successively with water and brine. The organic phase was dried over magnesium sulfate and the solvent was evaporated in vacuo to give the desired product (90%) as a pale brown oil.

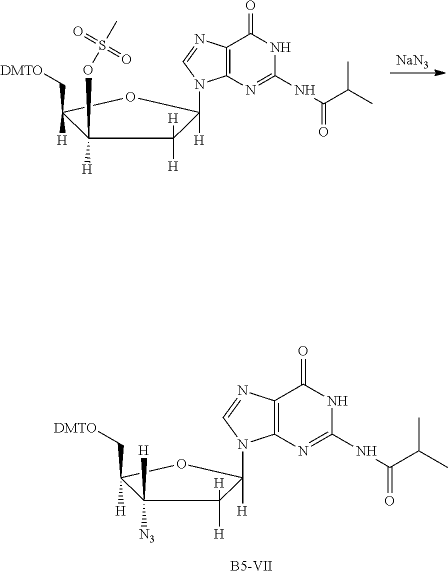

### Step 8—3′-(R)-(amino)²N-Isobutyryl-543-Dimetoxytrityl deoxyguanosine (B5-VIII)

A solution of compound (B5-VII) (0.12 mmol) in EtOH was exposed to a positive pressure of hydrogen gas at room temperature for 4 h in the presence of Pd black. The catalyst was removed by filtration on Celite and the filtrate was evaporated to dryness. The crude was purified by flash chromatography [1% NH₃(aq.)/MeOH] to afford after vacuum drying a white solid corresponding to the desired compound (81%).

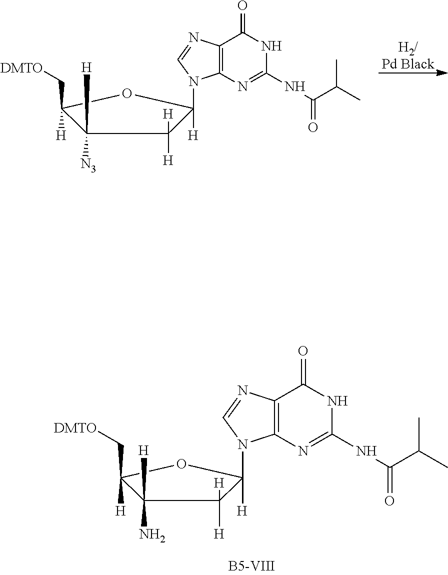

### Step 9—3′-(R)-[amino-(fmoc-aminoacid)]-²N-Isobutyryl-5′O-Dimetoxytrityl deoxyguanosine (B5-IX)

1 equivalent of the desired Fmoc-protected amino acid was suspended in 100 ml DMF and the mixture was cooled in an ice bath. To the suspension, 1.5 equivalent of dicyclohexyl carbodiimide (DCC) and 1.1 equivalent of 1-hydroxy benztriazole (HOBT) were added. The reaction mixture was stirred at 0° C. for 30 minutes and then 1 equivalent compound (B5-VIII) dissolved in 50 ml DMF was added in portions. The reaction temperature was elevated to r.t. and the mixture was stirred for 48 hours. The solvents were evaporated and the residue partitioned between DCM and DDW (50 mL each). The organic phase was washed three times with water, dried over sodium sulfate and evaporated to dryness. The crude was purified by flash chromatography using a gradient of petroleum ether to 20% ethyl acetate/petroleum ether.

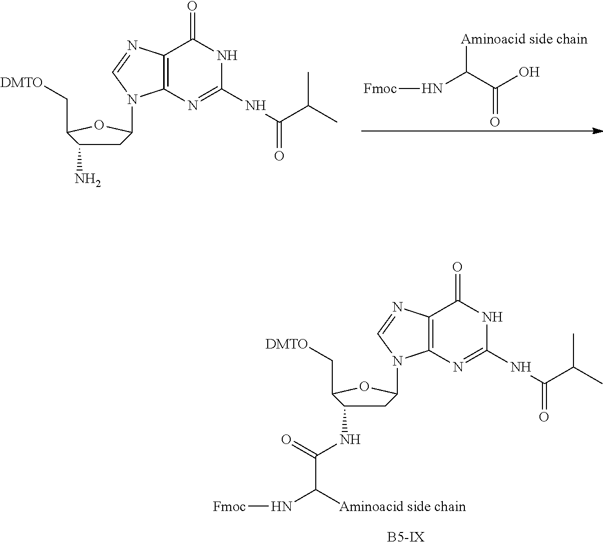

### Step 10—3′-(R)-[amino-(fmoc-aminoacid)]-²N-Isobutyryl-deoxyguanosine (B5-X)

Compound (B5-IX) was dissolved by the addition of 3% trichloroacetic acid in dry CH₂Cl₂. After stirring for ten minutes the reaction mixture was applied to a column of silica gel packed in CH₂Cl₂. The elution was performed with CH₂Cl₂/MeOH (93:7 v/v). The appropriate fractions were pooled and concentrated to give the pure desired compound as white powder.

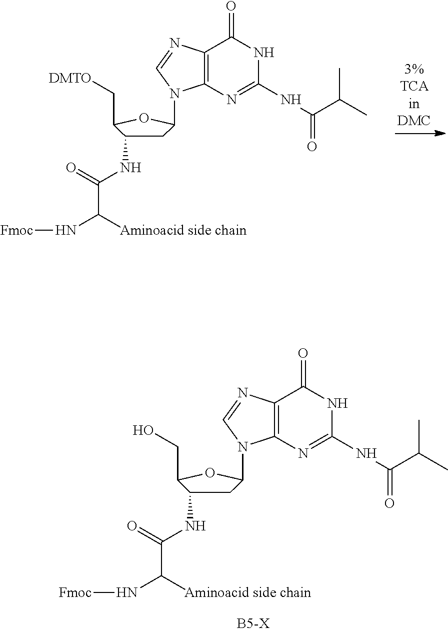

### Step 11—3′-(R)-[amino-(fmoc-aminoacid)]-²N-Isobutyryl-5′-O-methylene-(bisphosphonate)-deoxyguanosine (B5-XI)

A solution of methylenebis (phosphonic dichloride) (300 mg, 1.2 mmol) in trimethyl phosphate (10 mL) cooled to 0° C. was added to a suspension of compound (B5-X) (0.7 mmol) in trimethyl phosphate (10 mL) at 0° C. The reaction mixture was stirred at 0° C. After 1 h, 0.7 M aqueous TEAB (pH 7.0) was added. Chromatographic purification on DEAE-Sephadex, using a 0-1 M gradient of TEAB, gave a glassy solid.

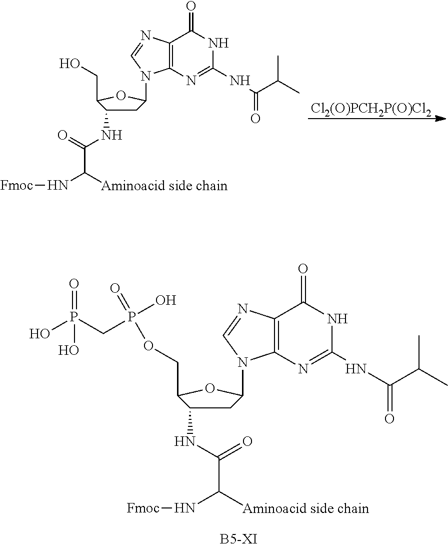

### Step 12—3′-(R)-[amino-(amino acid)]-5′-O-methylene-(bisphosphonate)-deoxyguanosine (B5)

Compound (B5-XI) was subjected to hydrolysis overnight at 60° C. in 25% ammonium hydroxide. After lyophilized, the crude was applied to a SAX HPLC semipreparative column. The desired product was obtained as a white powder.

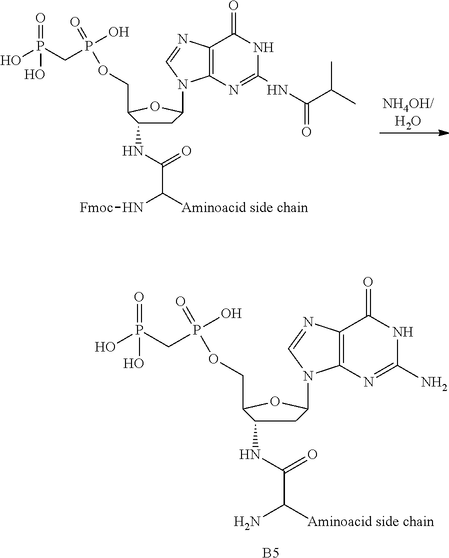

### Preparation of B3b

### Step 1—3′-(R)-(azido)²N-Isobutyryl-deoxyguanosine (B3b-I)

The procedure is the same as used for compound (B5-IX), using compound (B5-VII) as starting material.

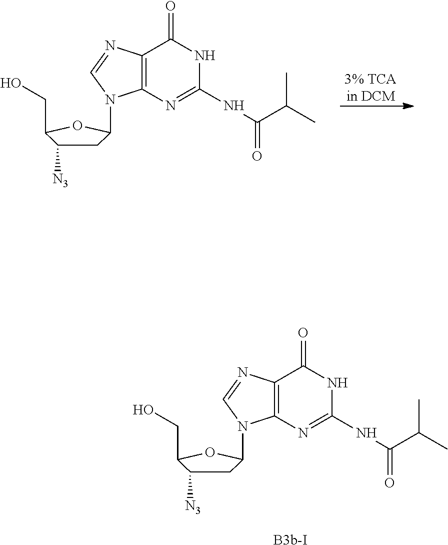

### Step 2—3′-(R)-(azido)²N-Isobutyryl-5′-O-methylene-(bisphosphonate)-deoxyguanosine (B3b-II)

The procedure is the same as used for compound (B5-XI), using compound (B3b-I) as starting material.

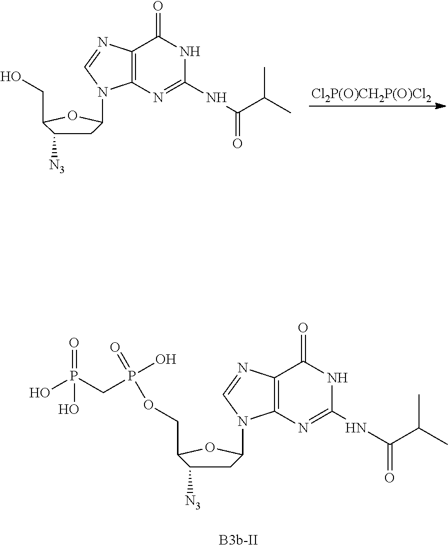

### Step 3—3′-(R)-(azido)-5′-O-methylene-(bisphosphonate)-deoxyguanosine (B3-b)

The procedure is the same as used in the last step of compound (B5), using compound (B3b-II) as starting material.

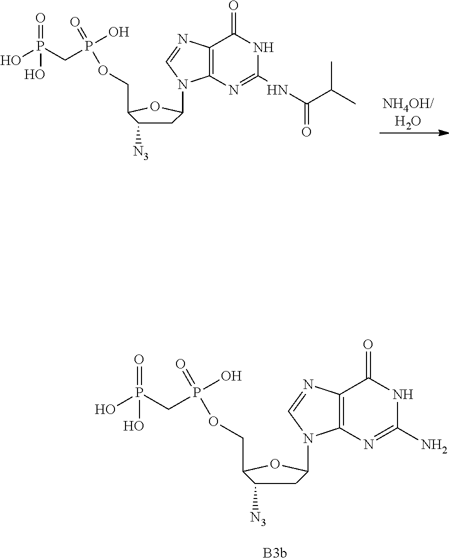

### Group C: 2′/3′ Sulfamic Acid Derivatives

### Preparation of C2 where Y═O and X═OH

### Step 1—²N-Isobutyryl-5′O-Dimetoxytrityl-3′O-tertbutyldimethylsilyl-2′-sulfamoyl-Guanosine (C₂-I)

A solution of (A9-IV) (0.47 mmol) in chloroform (5 mL) was treated with 2,6-di-t-butyl-4-methyl pyridine (DBMP) (1.42 mmol) and then sulfamoyl chloride (2.37 mmol). The reaction mixture was stirred for 19 h and then diluted with ethyl acetate (50 mL) and water (40 mL). The separated organic layer was then washed with brine (3×40 mL), dried, and evaporated. Column chromatography (3:1 hexane/ethyl acetate) gave a white solid.

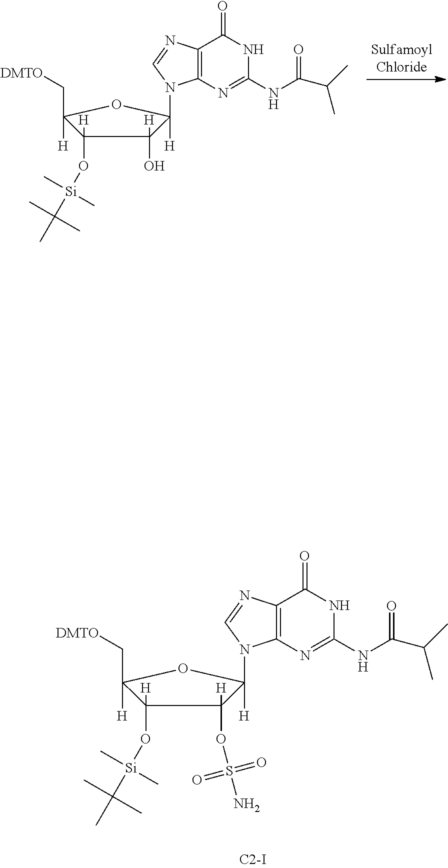

### Step 2—²N-Isobutyryl-3′-O-tertbutyldimethylsilyl-2′-sulfamoyl-Guanosine (C2-II)

Compound (C2-II) was dissolved by the addition of 3% dichloroacetic acid in dry CH₂Cl₂. After stirring for ten minutes the reaction mixture was applied to a column of silica gel packed in CH₂Cl₂. The elution was performed with CH₂Cl₂/MeOH (93:7 v/v). The appropriate fractions were pooled and concentrated to give the pure desired compound as white powder.

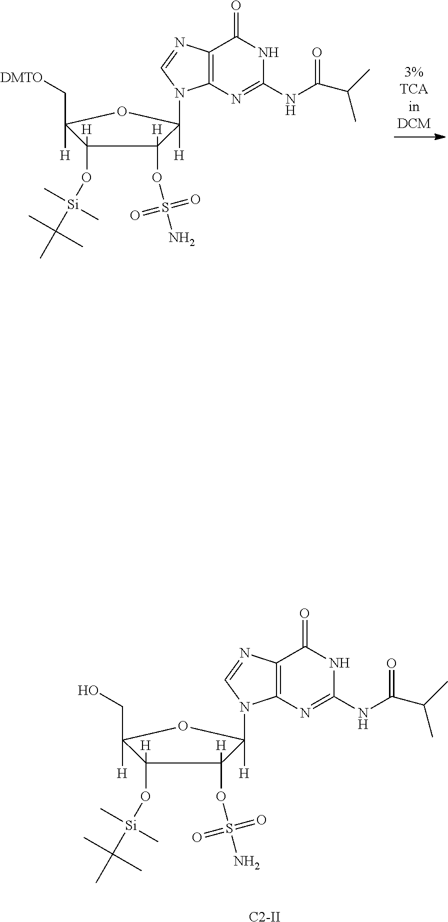

### Step 3—²N-Isobutyryl-5′ diphosphate-2′-sulfamoyl-Guanosine (C2-III)

A solution of pyrophosphoryl tetrachloride (1.2 mmol) in trimethyl phosphate (10 mL) cooled to 0° C. was added to a suspension of compound (C2-II) (0.7 mmol) in trimethyl phosphate (10 mL) at 0° C. The reaction mixture was stirred at 0° C. After 1 h, 0.7 M aqueous TEAB (pH 7.0) was added. Chromatographic purification on DEAE-Sephadex, using a 0-1 M gradient of TEAB, gave a white solid.

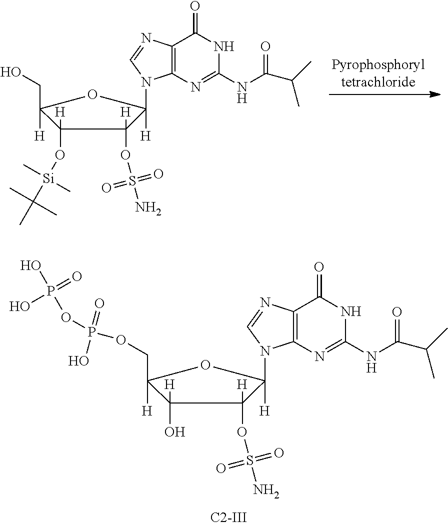

### Step 4—5′-diphosphate-2′-sulfamoyl-Guanosine (C2)

Compound (C2-III) was subjected to hydrolysis overnight at 60° C. in 25% ammonium hydroxide. After lyophilized, the crude was applied to a SAX HPLC semipreparative column. The desired product was obtained as a white powder.

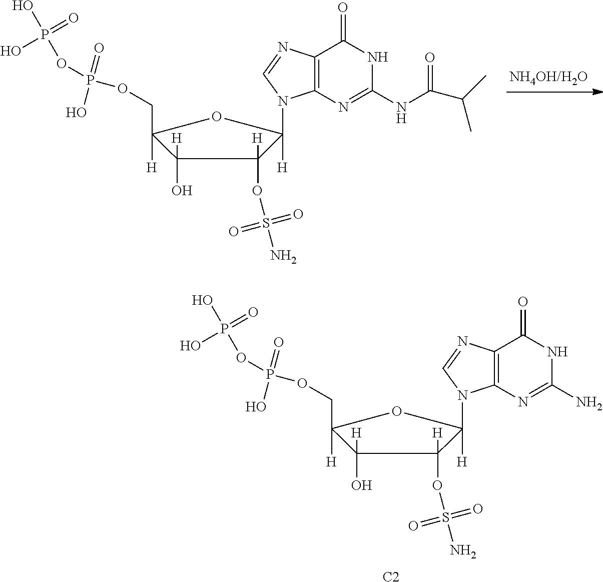

### Preparation of C6 where Y═O and X═OH

### Step 1—²N-Isobutyryl-5′-O-Dimetoxytrityl-3′-O-tertbutyldimethylsilyl-2′-O-[N-(Fmoc-amino acid)]-sulfamoyl-Guanosine (C6-I)

N-Fmoc protected amino acid (1.30 mmol) was added to a solution of compound (C2-I) (1.30 mmol), DCC (1.30 mmol), and DMAP (1.30 mmol) in dry dichloromethane (20 mL), and the mixture was stirred at room temperature for 2 h. The reaction mixture was diluted with ethyl acetate (150 mL), washed with sat. aqueous NaHCO₃, water, brine, dried (MgSO₄), and evaporated. The crude product was dissolved in MeOH/n-butylamine (10 mL/10 mL) and stirred at room temperature for 3 h. The solvents were evaporated and the crude product was purified by flash chromatography (EtOAc to 10% MeOH/EtOAc) to give (C6-I) as a white solid:

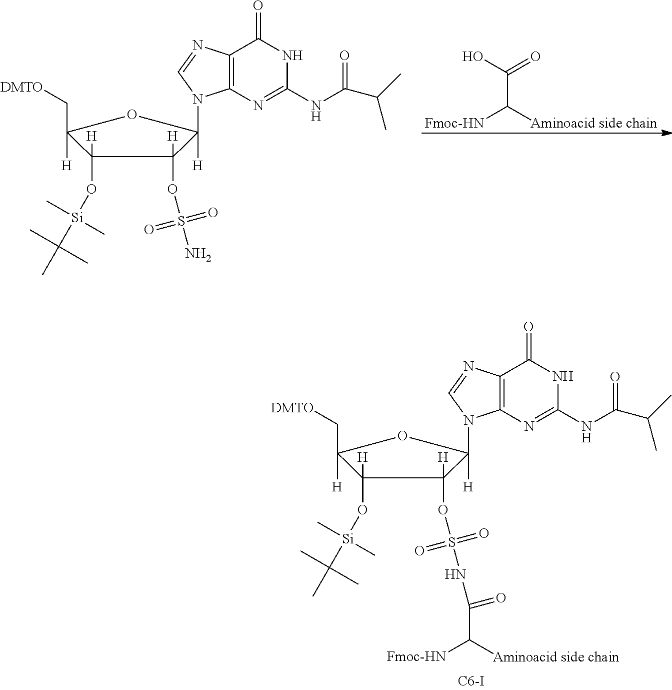

### Step 2—²N-Isobutyryl-3′-O-tertbutyldimethylsilyl-2′-O-[N-(Fmoc-amino acid)]-sulfamoyl-Guanosine (C6-II)

Same procedure as used for the preparation of compound (C2-II), using compound number (C6-I) as starting material.

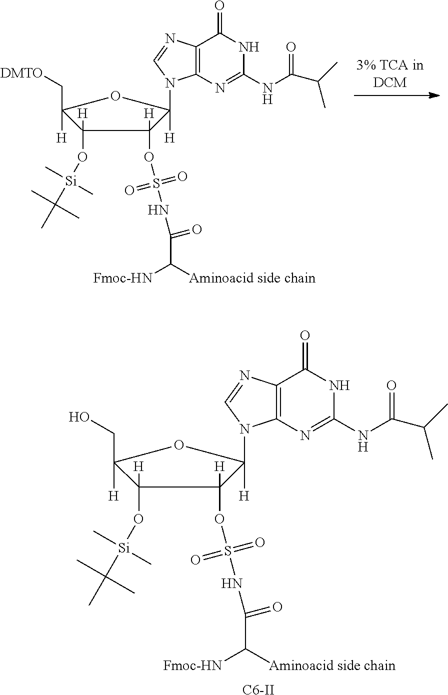

### Step 3—²N-Isobutyryl-5′-diphosphate-2′-O-[N-(Fmoc-amino acid)]-sulfamoyl-Guanosine (C6-III)

Same procedure as used for the preparation of compound number (C2-III), using compound number (C6-II) as starting material.

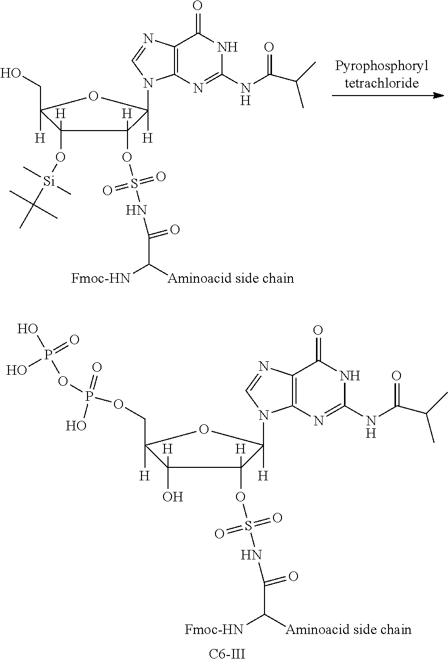

### Step 4—5′-diphosphate-2′-O-[N-(amino acid)]sulfamoyl-Guanosine (C6)

Same procedure as used in the last step of the preparation of compound (C2), using compound number (C6-III) as starting material.

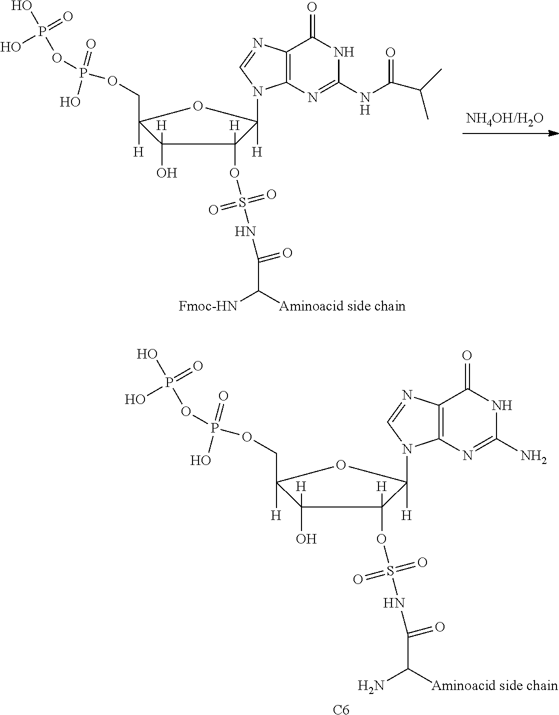

### Group D: ppGpp Analogs

### Preparation of D1 where Y═O, X═OH and Z═BHCN

### Step 1—²N-Isobutyryl-2′O-tertbutyldimethylsilyl Guanosine (D1a-I)

One gram of compound (A9-III) was dissolved by the addition of 3% dichloroacetic acid in dry CH₂Cl₂ (20 mL). After stirring for ten minutes the reaction mixture was applied to a column of silica gel packed in CH₂Cl₂. The elution was performed with CH₂Cl₂/MeOH (93:7 v/v). The appropriate fractions were pooled and concentrated to give pure ²N-Isobutyryl-2′O-tertbutyldimethylsilyl Guanosine (D1a-I) as white powder.

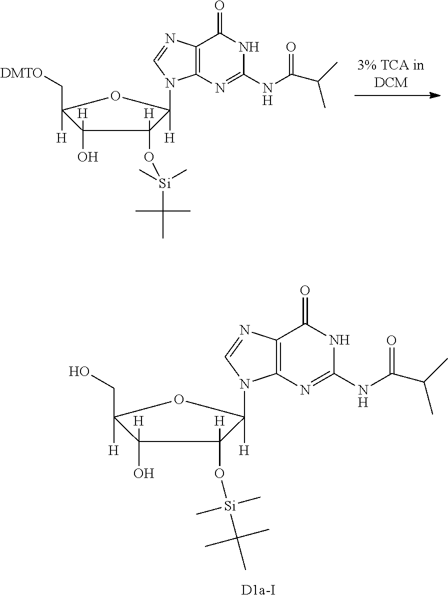

### Step 2—²N-Isobutyryl-2′O-tertbutyldimethylsilyl-3′,5′-O-di-(hydrogen phosphonate) guanosine (D1a-II)

To a stirred solution of phosphorus trichloride (0.4 mL, 4.53 mmol) in dry CH₂Cl₂ (10 mL) at room temperature under N₂, a solution of imidazol (0.92 g, 13.6 mmol) in dry CH₂Cl₂ (10 mL) was added. The solution was stirred for 30 min.

A solution of (D1a-I) (0.95 g, 2 mmol) and tetrazole (0.3 g, 4 mmol) in dry CH₂Cl₂ (10 mL) was added dropwise over a period of 10 min and the reaction mixture was stirred for an additional hour, followed by hydrolysis by 20 mL H₂O and extraction. The aqueous layer was concentrated and purified by semi-preparative strong anion exchange HPLC. The crude was eluted using a linear gradient of buffer (ammonium acetate 0<0.5 M). The appropriate fractions were collected and lyophilized, to obtain the desired compound.

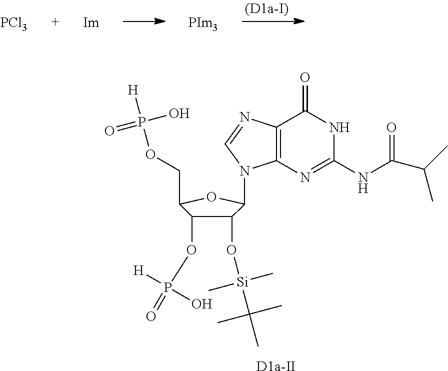

### Step 3 ²N-Isobutyryl-2′O-tertbutyldimethylsilyl-3′,5′-O-di-(α-cyanoboro-phosphate)-guanosine (D1a-III)

To 1 mmol of compound (D1a-II) dissolved in dry THF, a solution of sodium cyanoborohydride (1 mmol) was added and stirred for 48 hours. The precipitated product was filtered and dried under vacuum.

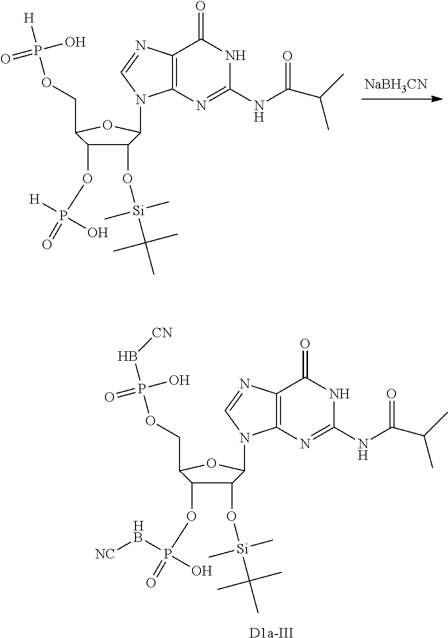

### Step 4—²N-Isobutyryl-2′O-tertbutyldimethylsilyl-3′,5′-O-di-[(α-cyanoboro-phosphate),(β-hydrogen phosphonate)]-guanosine (D1a-IV)

Same procedure as used for the preparation of compound (D1a-II), using compound (D1a-III) as starting material.

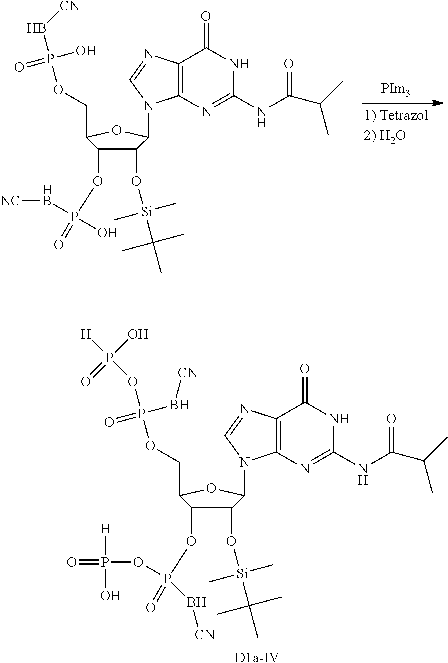

### Step 5—²N-Isobutyryl-2′O-tertbutyldimethylsilyl-3′,5′-O-di-[(α-cyanoboro-phosphate),(β-phosphate)]-guanosine (D1a-V)

Compound (D1a-IV) was dissolved in 20 mL of 2% I₂ in pyridine:H₂O (9:1) and stirred at room temperature for 36 hours. When ³¹P NMR showed the disappearance of ¹H-³¹P coupling the mixture was evaporated, dissolved in water and washed with CH₂Cl₂. The aqueous layer was concentrated and purified by semi-preparative strong anion exchange HPLC. The crude was eluted using a linear gradient of buffer (ammonium acetate 0<0.5 M). The appropriate fractions were collected and lyophilized, to afford (D1a-V)

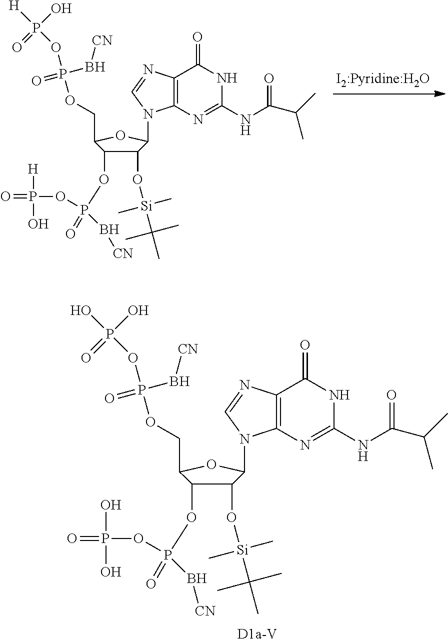

### Step 6—2′O-tertbutyldimethylsilyl-3′,5′-O-di-[(α-cyanoboro-phosphate),(β-phosphate)]-guanosine (D1a-VI)

Same procedure as used in the last step of the preparation of compound number (C2), using compound number (D1a-V) as starting material.

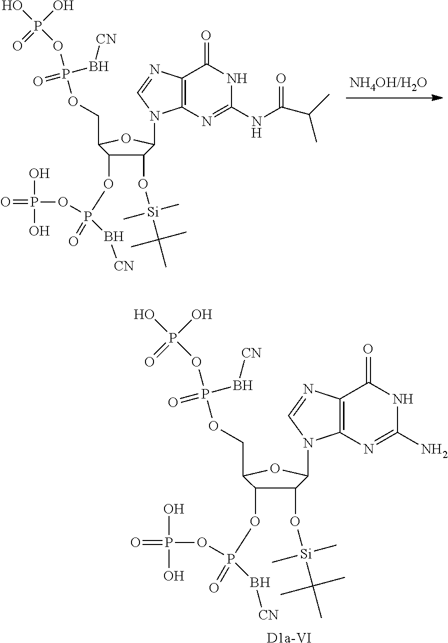

### Step 7—3′,5′-O-di-[(α-cyanoboro-phosphate),(β-phosphate)]-guanosine (D1a)

Compound (D1a-VI) was dissolved in THF. A 1M solution of tetrabutyl ammonium fluoride in THF was added. The mixture was stirred for one hour and the solvents evaporated to dryness. The crude was applied to a SAX HPLC semipreparative column. The desired product was obtained as a white powder.

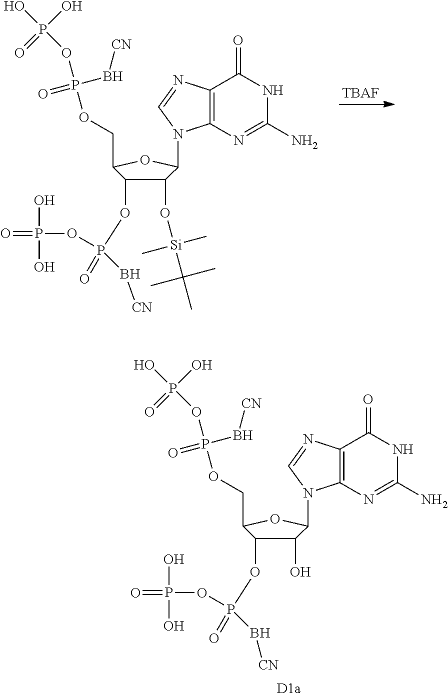

### Preparation of D1 where Y═O, X═OH and Z═O(CH₂)₃N(CH₃)₂ (D1b)

### Step 1—²N-Isobutyryl-2′-O-tertbutyldimethylsilyl-3′,5′-O-di-(α-cyanoethyl-hydrogen phosphonate)-guanosine (D1b-1)

Compound (D1a-I) was dried by co-evaporation with dry toluene and suspended in dry pyridine (10 mL) under inert atmosphere. Diphenyl phosphite was added and stirred for 2 h. 3-hydroxypropionitrile was added. After stirring for 2 hr, the solvent was evaporated. The oily crude was used without further purification.

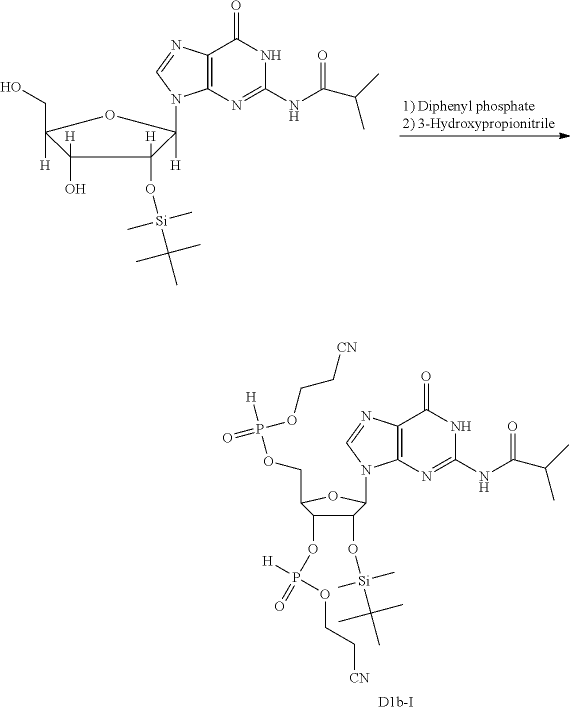

### Step 2—2′-O-tertbutyldimethylsilyl-3′,5′-O-di-[α-O-(N,N-dimethyl)-propylamino-phosphate]-guanosine (D1b-II)

Compound (D1b-I) was dissolved in 10 mL of a solution of CCl₄ in pyridine (50%) containing 10% (v/v) of N,N-dimethyl-3-aminopropanol. The mixture was stirred for 30 minutes and the solvents were evaporated.

The presence of the desired compound was detected by ESI-MS. The crude was subjected to hydrolysis overnight at 60° C. in 25% ammonium hydroxide. After lyophilized, the crude was applied to a SAX HPLC semipreparative column. The desired product was obtained as a white powder.

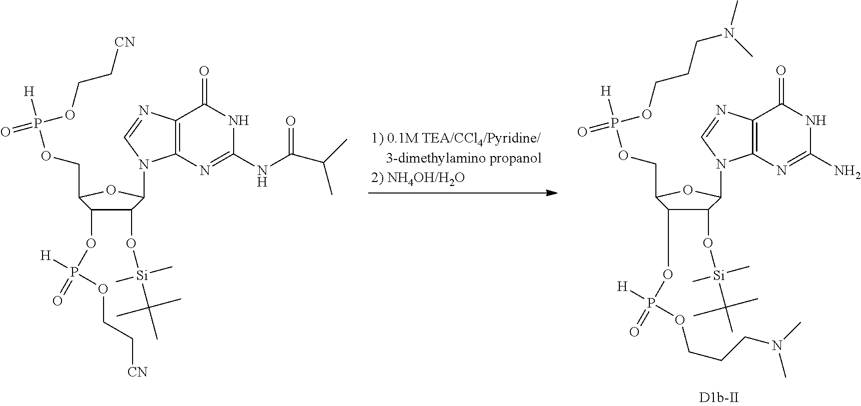

### Step 3—2′-O-tertbutyldimethylsilyl-3′,5′-O-di-{[α-O-(N,N-dimethyl)-propylamino-phosphate],(β-hydrogen phosphonate)}-guanosine (D1b-III)

Same procedure as used for the preparation of compound (D1a-II), using compound (D1b-II) as starting material.

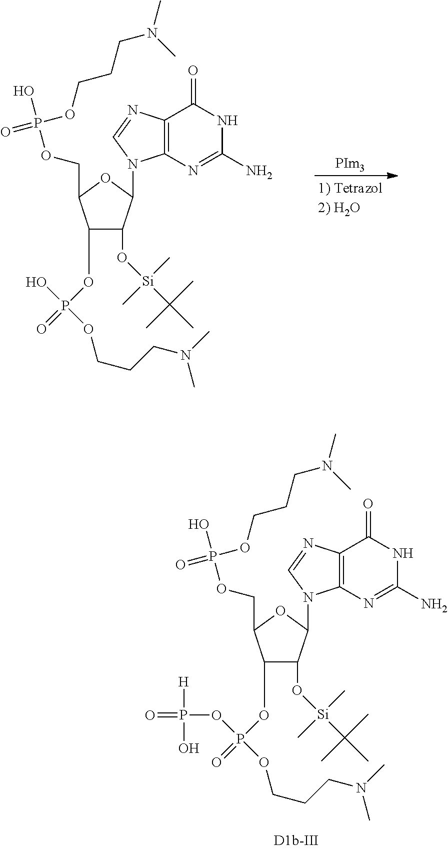

### Step 4—2′-O-tertbutyldimethylsilyl-3′,5′-O-di-{[α-O-(N,N-dimethyl)-propylamino-phosphate],(β-phosphate)}-guanosine (D1b-IV)

Same procedure as used for the preparation of compound (D1a-V), using compound (D1b-III) as starting material.

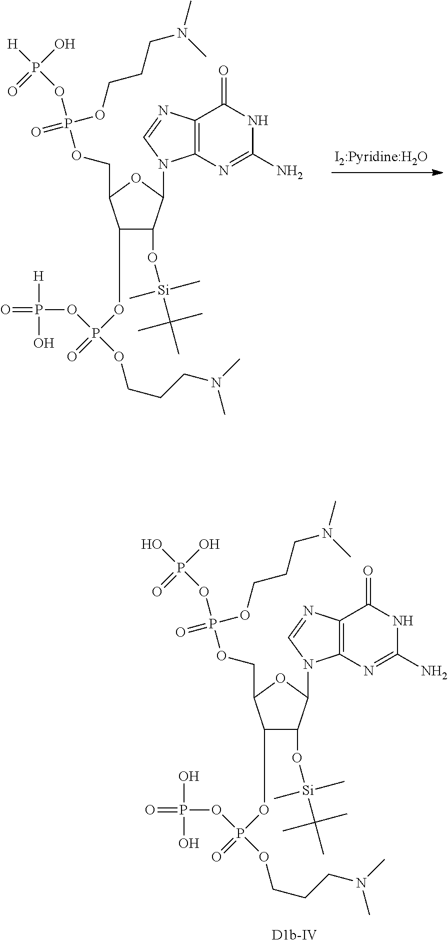

### Step 5—3′,5′-O-di-{[α-O-(N,N-dimethyl)-propylamino-phosphate],(β-phosphate)}-guanosine (D1b)

Same procedure as used in the last step of the preparation of compound (D1a), using compound number (D1b-IV) as starting material.

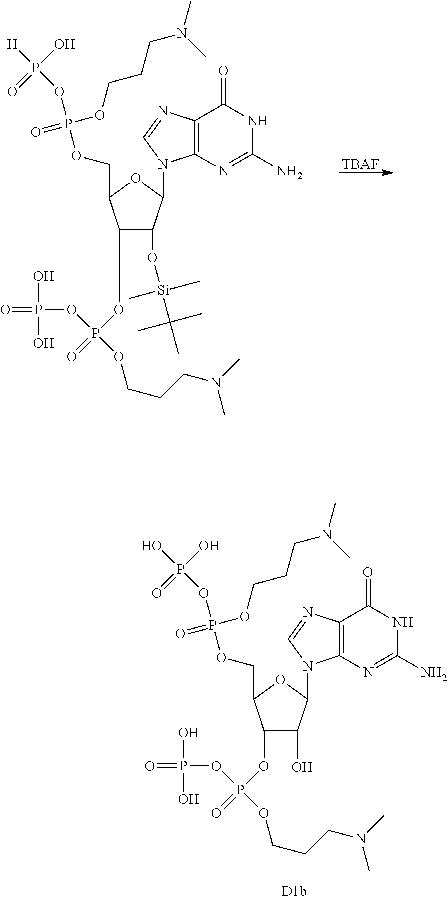

### Preparation of D3

### 5′,3′-O-di-[methylene-(bisphosphonate)]-2′-deoxyguanosine

A solution of methylenebis (phosphonic dichloride) (1.6 mmol) in trimethyl phosphate (10 mL) cooled to 0° C. was added to a suspension of dried T-deoxyguanosine (0.7 mmol) in trimethyl phosphate (10 mL) at 0° C. The reaction mixture was stirred at 0° C. overnight. Then 1M aqueous ammonium bicarbonate (pH 7.0) was added. Chromatographic purification on DEAE-Sephadex, using a 0-1 M gradient of ammonium bicarbonate, and then SAX HPLC gave a glassy solid.

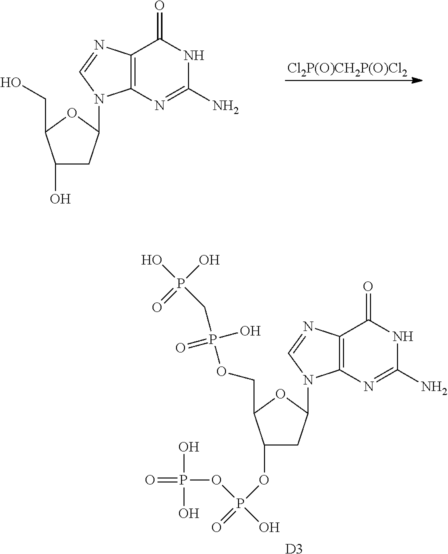

### Group E: 2′3′ Cyclic Derivatives

### Preparation of E3a

### Step 1=2′, 3′-Isopropylideneguanosine (E3a-I)

To a suspension of guanosine (10 g, 35.31 mmol) in 600 ml of acetone was added 70% perchloric acid (4.1 ml, 47.54 mmol). After 70 minutes, concentrated ammonium hydroxide (6.7 ml, 49.79 mmol) was added to the reaction mixture and cooled down with ice-water bath. The solid was filtered out and dried over vacuum, 9.5 g (83.2%).

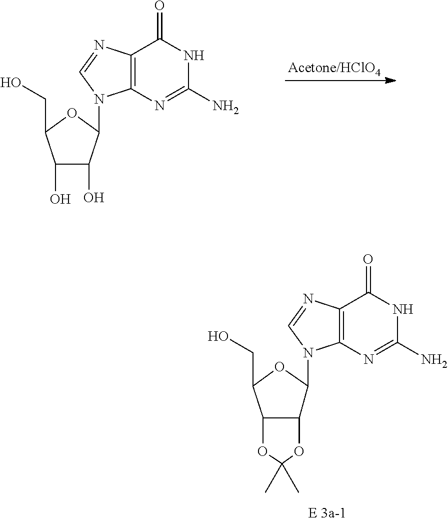

### Step 2—5′-pyrophosphate,2′,3′-Isopropylideneguanosine (E3a)

A solution of pyrophosphoryl tetrachloride (1.2 mmol) in trimethyl phosphate (10 mL) cooled to 0° C. was added to a suspension of compound (E3a-I) (0.7 mmol) in trimethyl phosphate (10 mL) at 0° C. The reaction mixture was stirred at 0° C. After 1 h, 0.7 M aqueous ammonium bicarbonate (pH 7.0) was added. Chromatographic purification on DEAE-Sephadex, using a 0-1 M gradient of TEAB, gave a glassy solid.

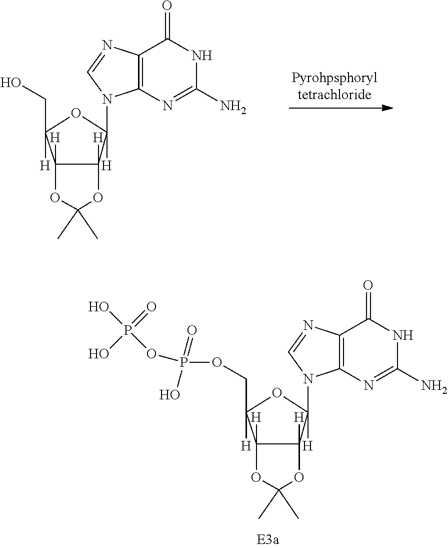

### Preparation of E5 where Y═CH₂

### Step 1—2′, 3′-Benzylideneguanosine (E5-I)

Guanosine was dissolved in benzaldehyde and stirred for three days in the presence of dry Zinc chloride. When the reaction was over, benzaldehyde was evaporated and the residue partitioned between dichloromethane and water.

The organic phase was washed three times with water, dried and evaporated to dryness. The crude was applied to a silica gel column and eluted with 5% methanol in chloroform.

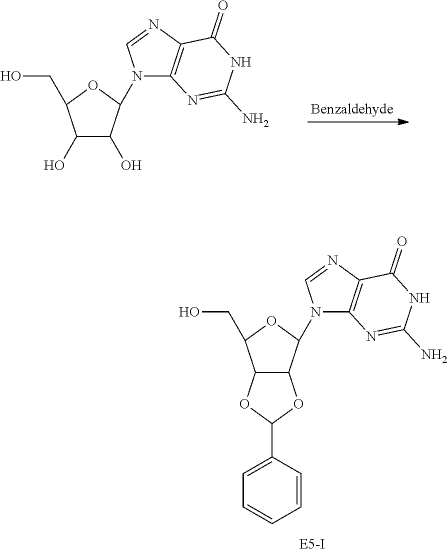

### Step 2—5′-O-methylene-(bisphosphonate)-2′,3′-Benzylideneguanosine (E5-II)

Same procedure as used for the preparation of compound (B5-IX) using compound (E5-I) as starting material.

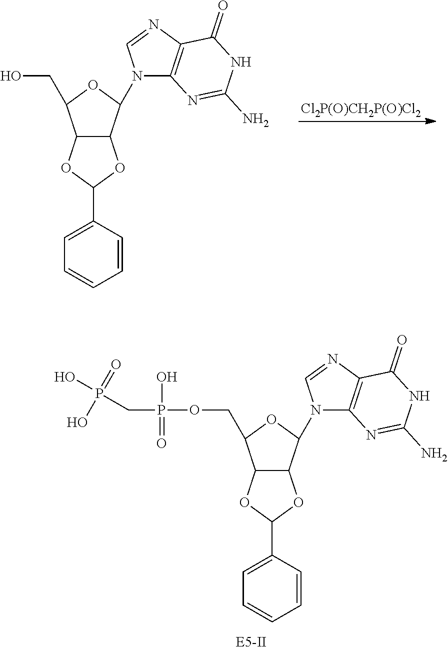

### Preparation of E2 where Y═CH₂

### Step 1—²N-Isobutyryl-5′O-Dimetoxytrityl-2′,3′[Cyclic-Hydrogen-phosphonate]Guanosine (E2-I)

To a stirred solution of 5′-O-dimethoxytrityl 2-isobutyril Guanosine (A9-II) (1 mmol; dried by repeated evaporation of added pyridine) in pyridine (10 mL) was added diphenyl H-phosphonate (1.5 molar equiv). After 20 min (31P NMR analysis), the solvent was evaporated and the crude containing the produced cyclic H-phosphonate was oxidized by dissolving it in 20 mL of 2% I2 in Py/H2O (9:1). After stirring for 30 minutes the desired compound was obtained.

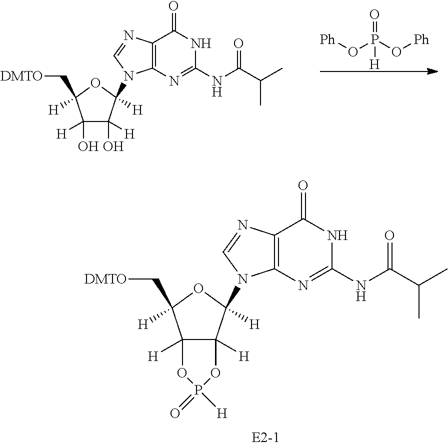

### Step 2—²N-Isobutyryl-2′,3′ [Cyclic-Hydrogen-phosphonate]Guanosine (E2-II)

Same procedure as used for the preparation of compound number (D1a-I), using compound number (E2-I) as starting material.

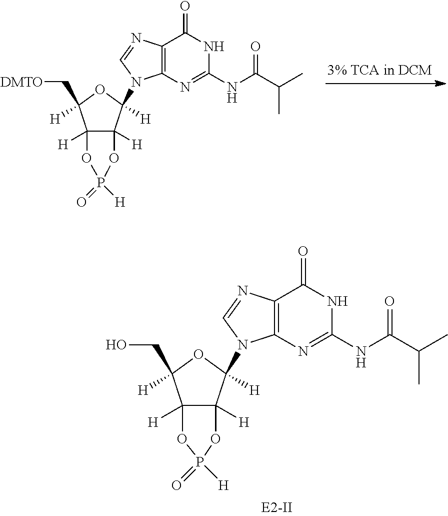

### Step 3—²N-Isobutyryl-5′-O-methylene-(bisphosphonate)-2′,3′[Cyclic-Hydrogen-phosphonate]Guanosine (E2-III)

Same procedure as used for the preparation of compound number (E5-II), using compound number (E2-II) as starting material.

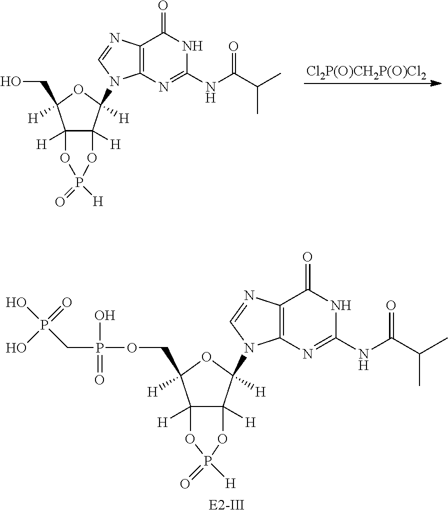

### Step 4—5′-O-methylene-(bisphosphonate)-2′,3′[Cyclic-Hydrogen-phosphonate]Guanosine (E2)

Same procedure as used for the preparation of compound number (D1a-VI), using compound number (E2-III) as starting material.

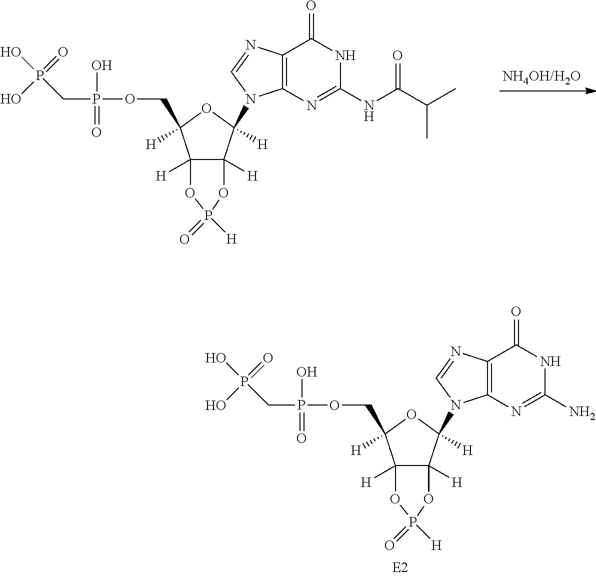

### Preparation of E1 where Y═CH₂

### 5′-O-methylene-(bisphosphonate)-2′,3′-[Cyclic-phosphate]-Guanosine (E1)

Same procedure as used for the preparation of compound number (D1b-IV), using compound number (E2) as starting material.

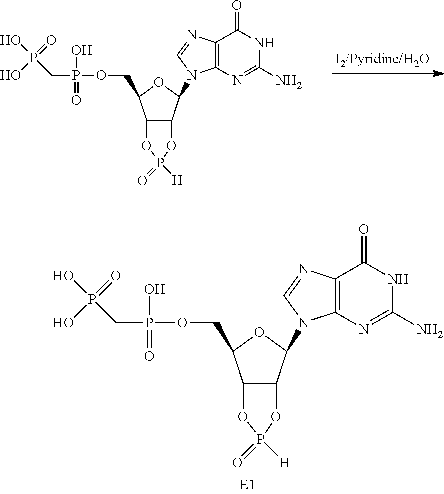

### EXAMPLE 2

### Experimental Procedures

### A. Cell Growing:

Starters of deltaRelA E. coli cells, expressing one of the following proteins (RelA, RelA638 or Relseq₃₈₅) in trans, were grown overnight at 37° C. The next day, the starters were diluted 1:50 in 400 ml LBamp and the cells have continued to grow at 37° C. until they reached O.D₆₀₀=˜0.6. Then, the cells were added with IPTG (1 mg/ml) and the cells were grown at the same conditions for additional 2-3 h. After that the cells were harvested for 10 min at 4000 rpm and the pellet was frozen at −80° C.

### Protein Purification:

Lysis of the pellet using “Lysis Buffer” containing Lysozyme (3 mg/ml) and ½ pill of Complete (EDTA-free).
Sonication on ice for 3.5 min.
Centrifuge the cells for 10 min at 10000 rpm.
Mix the supernatant with Ni-NTA bids for 1 h at 4° C.
Load the bids on a column and wash it with “Wash Buffer”.
Elute the protein using “Elution Buffer”.

The elution fractions were run on 12% Acryl/Bis gel and the fractions containing the most protein were taken to dialysis (two times overnight at 4° C.). After dialysis the concentration of the protein was determined using Bradford Reagent (O.D₅₉₅).

### Dialysis Buffer:

100 mM Tris-Ac pH8.5

10 mM EDTA

1 mM DTT

25% Glycerol

Final volume: 2 liter

In Vitro (p)ppGpp Accumulation:

### RMx5:

2.5 mM GTP, 20 mM ATP, 200 mM Tris HCl pH7.4, 5 mM DTT, 50 mM MgCl₂

50 mM KCl, 135 mM (NH₄)₂SO₄, α-³²P GTP (0.1 μl per reaction), DDW

Once the buffer is added together with 1 μg of protein, 0-10 mM of inhibitor and 30 μg of ribosomes (in a total volume of 20 μl) the following reaction occurs: GTP/GDP+ATP→(p)ppGpp.

The reactions are incubated at room temperature for a period of 10-90 minutes. Then, the reactions are stopped by the addition of 5 μl of Formic acid. Once the reactions were stopped, they were placed on ice until 5 μl of each reaction were loaded on Cellulose PEI (Merck) and ran for 2 hours in 1.5M KH₂PO₄. The PEI was read, using image reader-1000 V1.8 and the data was analyzed by TINA 2.0 software.

### EXAMPLE 3

### Results

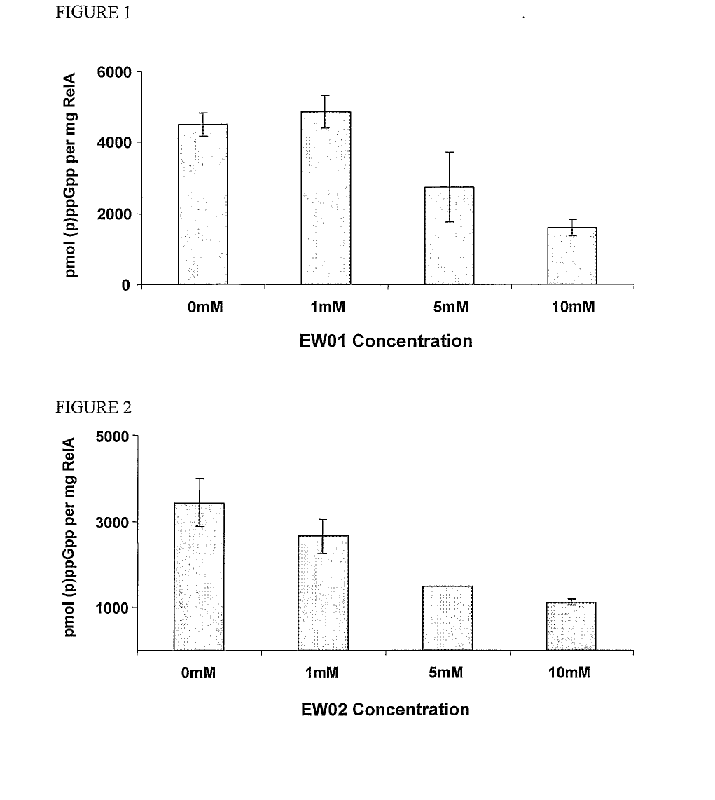

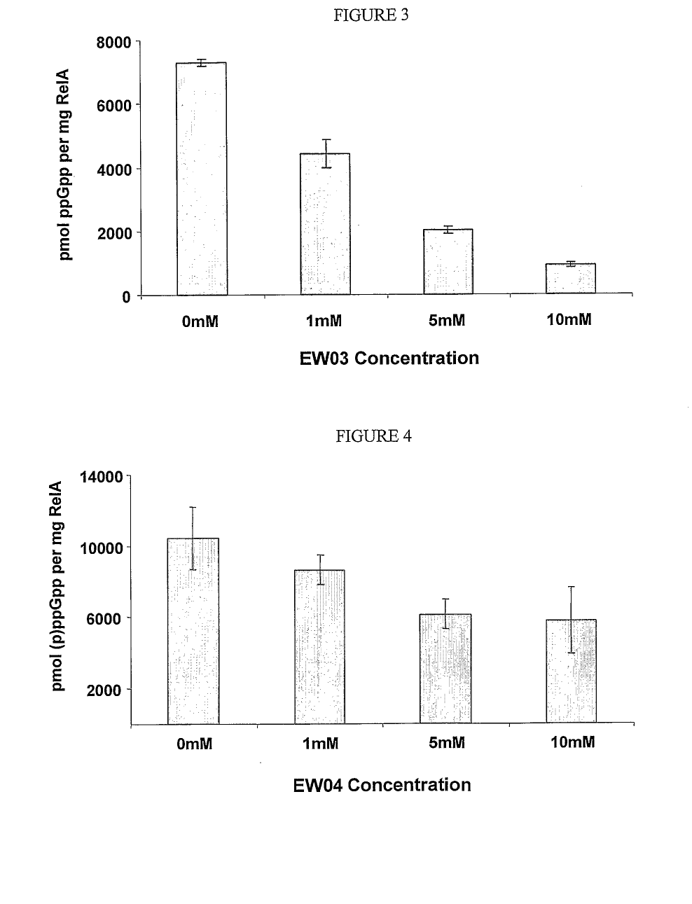

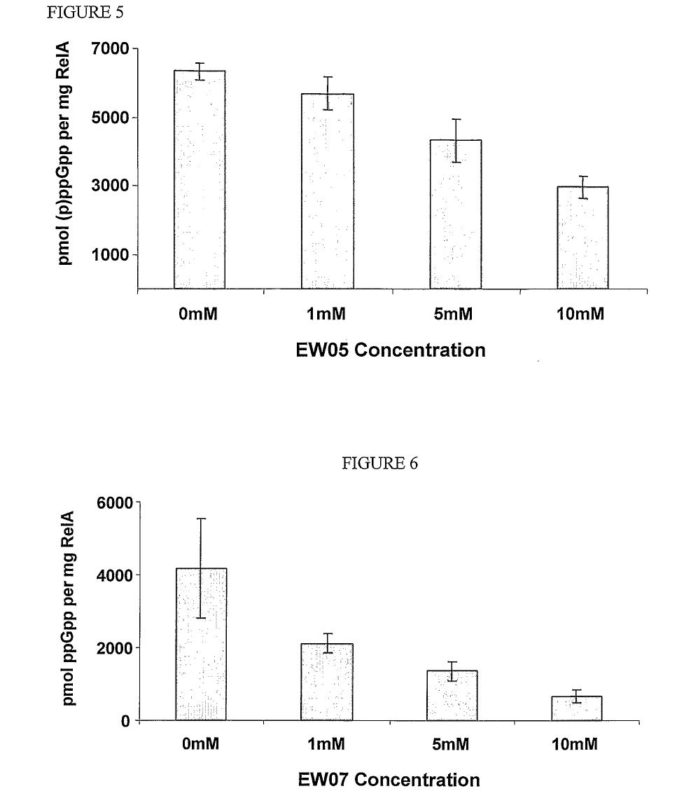

The effects of the newly purified (p)ppGpp analogues A1 (or EW01) (FIG. 1), E3b, i.e., E3 where Y═CH₂ (or EW02) (FIG. 2), D3 (or EW 03) (FIG. 3), D7 (or EW 04) (FIG. 4), D8 (or EW 05) (FIG. 5) or D6 (or EW 07) (FIG. 6) on Gram Negative E. coli RelA in vitro activity were examined. Each of the analogues were added and the effects of each compound on (p)ppGpp accumulation were measured. Results are presented as pmol (p)ppGpp per mg RelA vs. compound concentrations.

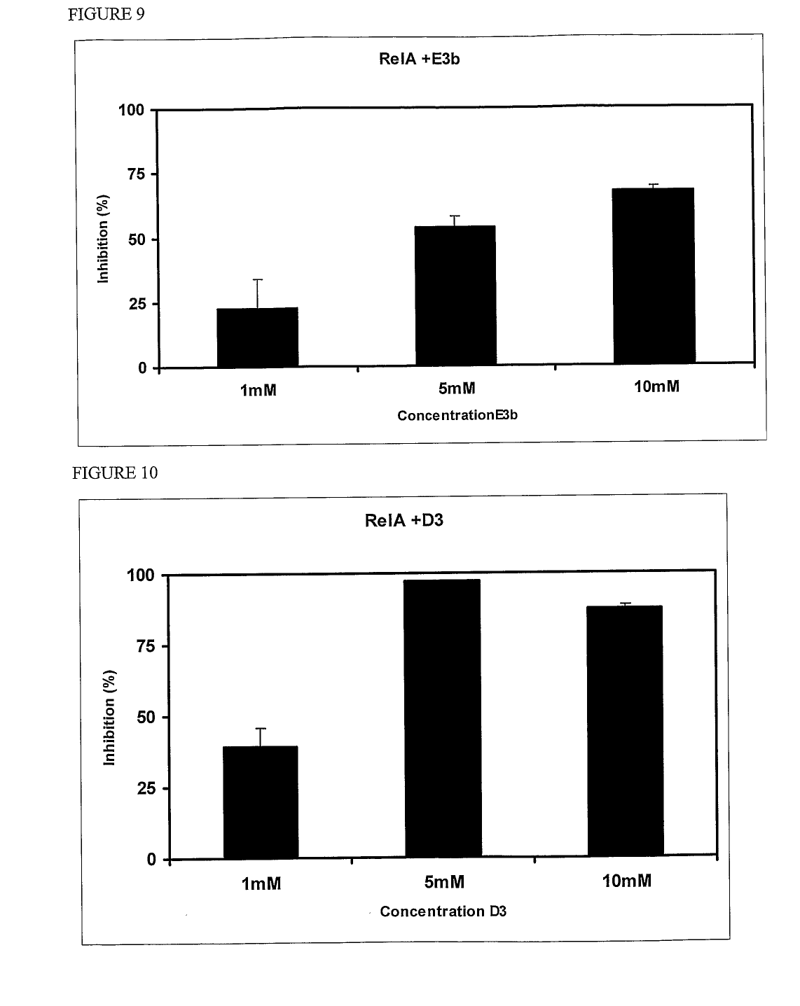

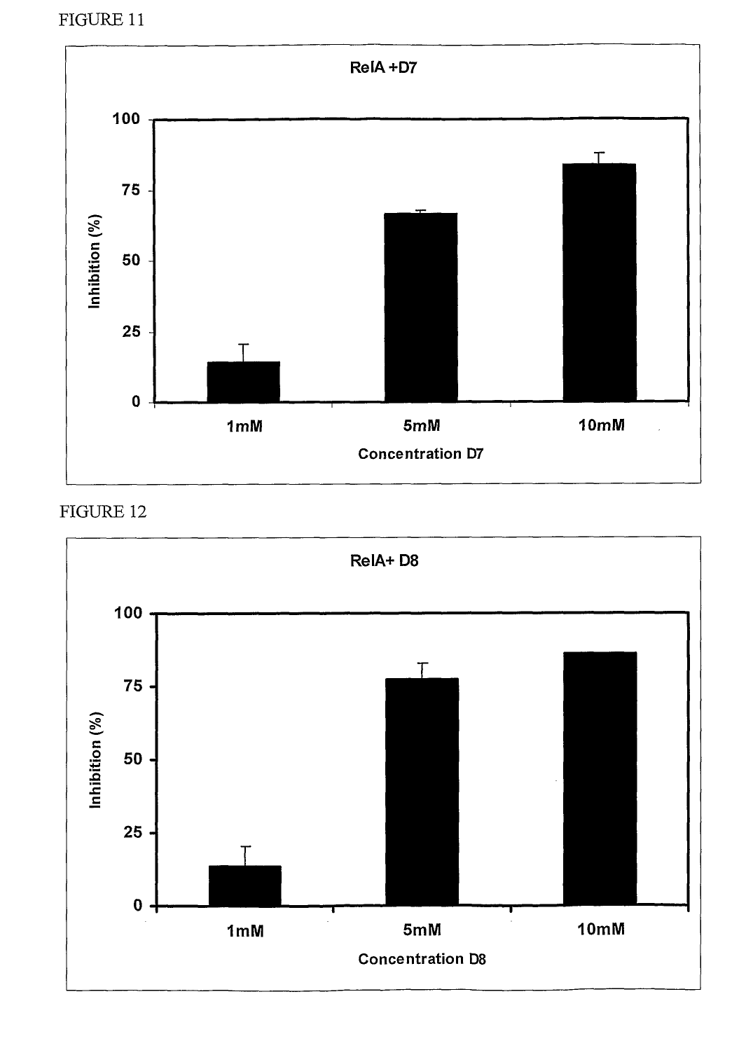

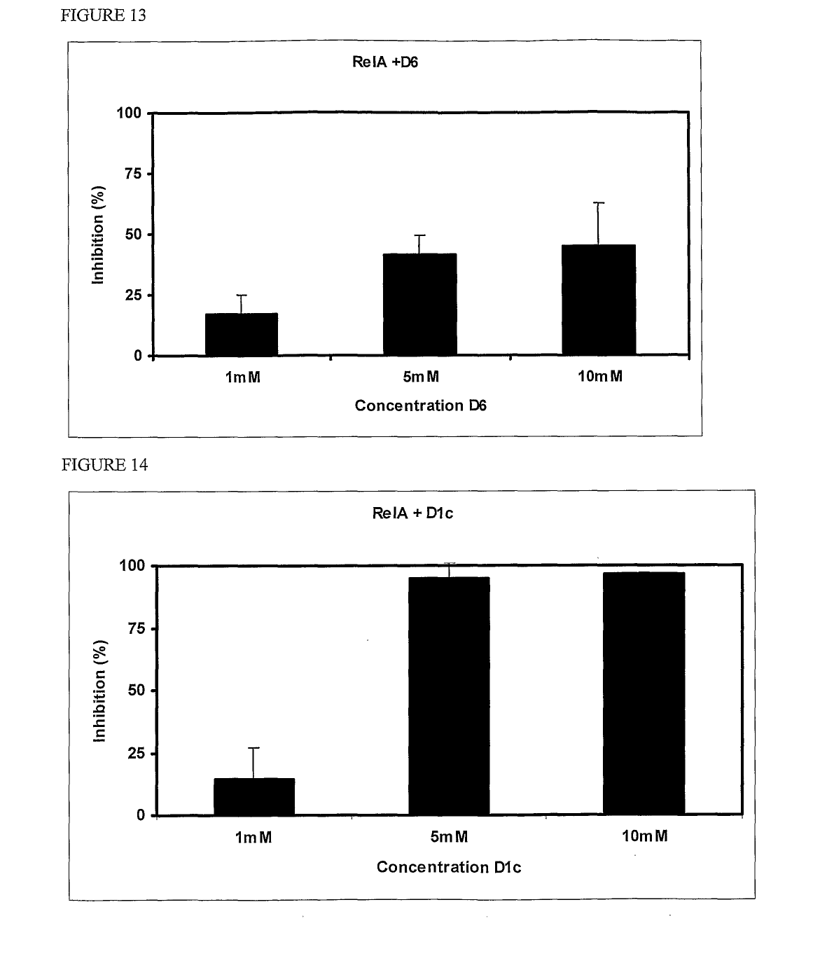

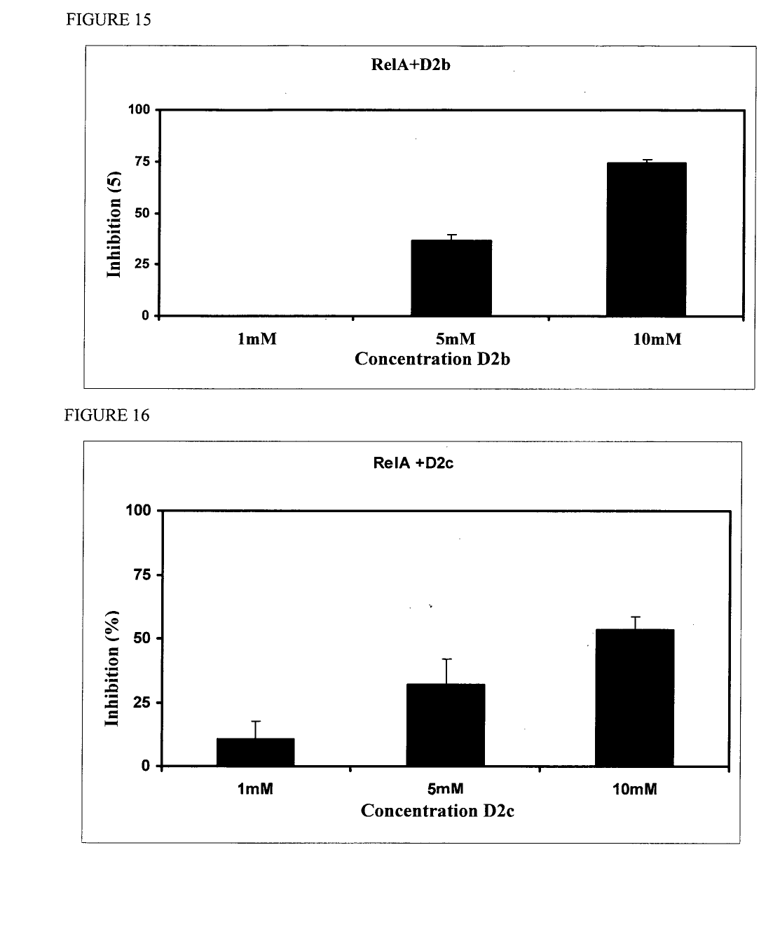

In a separate experiment, the effects of the (p)ppGpp analogues A1 (FIG. 8), E3b (FIG. 9), D3 (FIG. 10), D7 (FIG. 11), D8 (FIG. 12), D6 (FIG. 13), D1c (FIG. 14), D2b (FIG. 15) and D2c (FIG. 16) on Gram Negative E. coli RelA in vitro activity were examined. Each of the analogues were added and the effects of each compound on (p)ppGpp accumulation were measured. Results are presented % inhibition vs. compound concentration.

As seen, all of the tested compounds inhibited E. coli RelA in vitro activity at the concentrations tested.

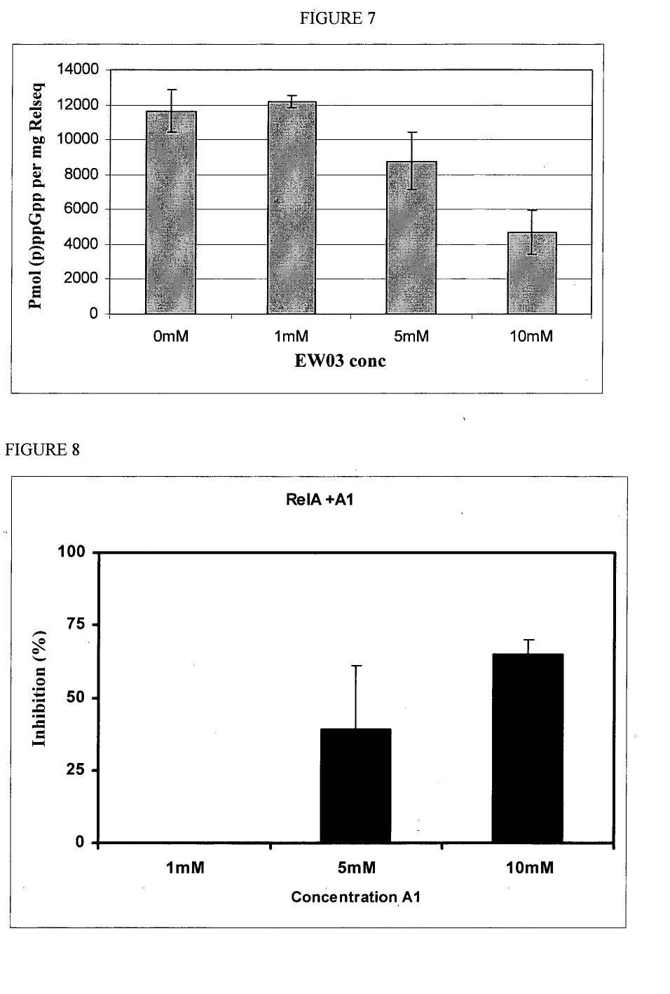

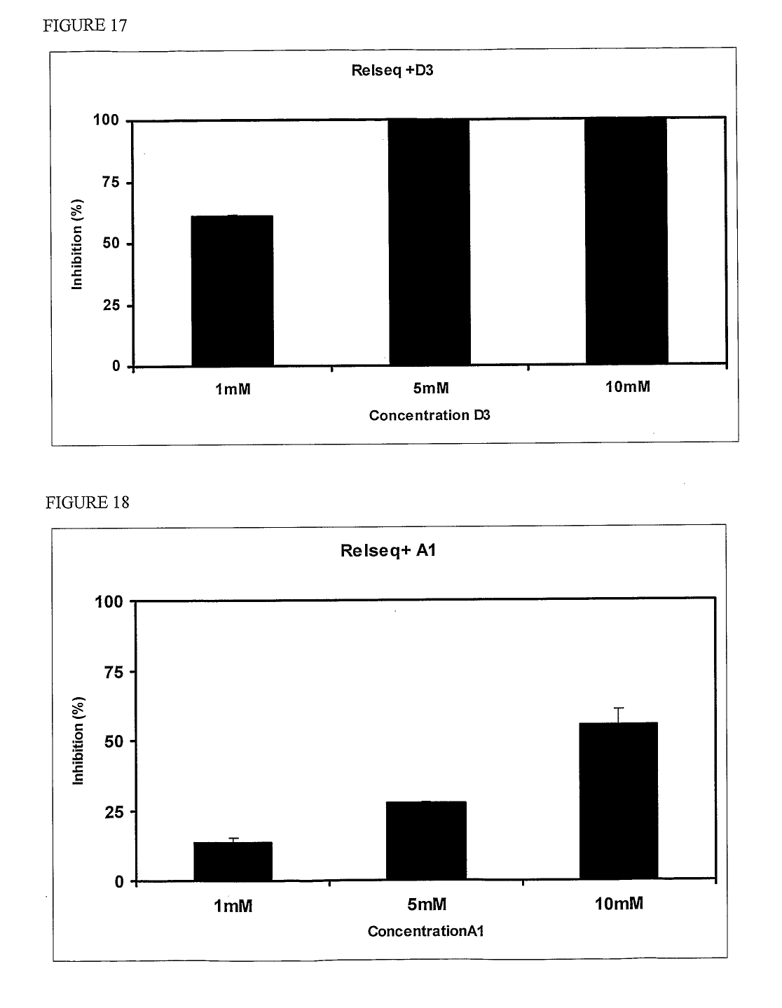

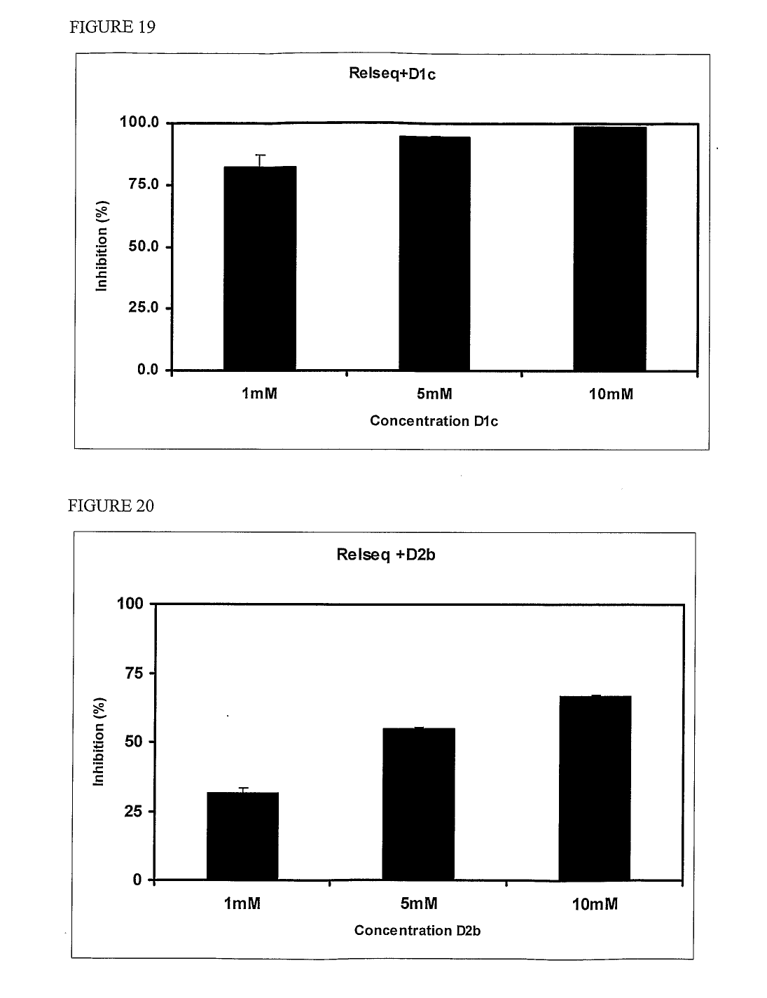

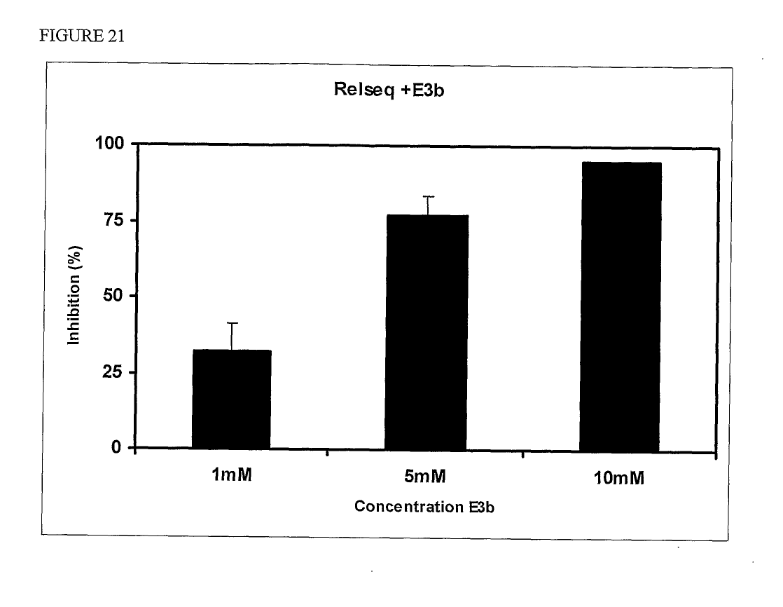

Furthermore, the effects of the newly purified (p)ppGpp analogue D3 (or EW03) (FIGS. 7 and 17), A1 (FIG. 18), D1c (FIG. 19), D2b (FIG. 20), and E3b (FIG. 21) on in vitro activity of Relseq from Gram Positive Streptococcus equisimils were examined. Each of the analogues were added and the effects of each compound on (p)ppGpp accumulation were measured. Results are presented as pmol (p)ppGpp per mg Relseq vs. compound concentrations (FIG. 7); or as % inhibition vs. compound concentration (FIGS. 17-21). As seen, all of the tested compounds inhibited Streptococcus equisimils Relseq in vitro activity at the concentrations tested.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

### REFERENCES

Cashel, M., and Gallant, J. (1969). Two compounds implicated in the function of the RC gene of Escherichia coli. Nature 221, 838-841.
Cashel, M., Lazzarini, R. A., and Kalbacher, B. (1969). An improved method for thin-layer chromatography of nucleotide mixtures containing ³²P-labelled orthophosphate. J. Chromatogr. 40, 103-9.
Cashel, M. G. D., Hernandez, V J, Vinella, D. (1996). The Stringent response. E. coli and Salmonella typhymurium: cellular and molecular biology, 2nd ed., pp. 1458-1496.
Gentry, D., Li, T., Rosenberg, M., and McDevitt, D. (2000). The rel gene is essential for in vitro growth of Staphylococcus aureus. J. Bacteriol. 182, 4995-4997.
Gentry, D. R., and Cashel, M. (1995). Cellular localization of the Escherichia coli SpoT protein. J. Bacteriol. 177, 3890-3893.
Gentry, D. R., Hernandez, V. J., Nguyen, L. H., Jensen, D. B., and Cashel, M. (1993). Synthesis of the stationary-phase sigma factor σ^{s} is positively regulated by ppGpp. J. Bacteriol. 175, 7982-7989.
Givens, R. M., Lin, M. H., Taylor, D. J., Mechold, U., Berry, J. O. and Hernandez, V. J. (2004). Inducible expression, enzymatic activity, and origin of higher plant homologues of bacterial RelA/SpoT stress proteins in Nicotiana tabacum. J. Biol. Chem. 279, 7495-7504.
Harris, B. Z., Kaiser, D., and Singer, M. (1998). The guanosine nucleotide (p)ppGpp initiates development and A-factor production in Myxococcus xanthus. Genes Dev. 12, 1022-1035.
Haseltine, W. A., and Block, R. (1973). Synthesis of guanosine tetra and penta phosphate requires the presence of a codon specific, uncharged transfer ribonucleic acid in the acceptor site of ribosomes. Proc. Natl. Acad. Sci. USA 70, 1564-1568.
Mechold, U., Cashel, M., Steiner, K., Gentry, D., and Malke, H. (1996). Functional analysis of a relA/spoT gene homolog from Streptococcus equisimilis. J. Bacteriol. 178, 1401-1411.
Mechold, U., and Malke, H. (1997). Characterization of the stringent and relaxed responses of Streptococcus equisimilis. Bacteriol. 179, 2658-2667.
Metzger, S., Dror, I. B., Aizenman, E., Schreiber, G., Toone, M., Friesen, J. D., Cashel, M., and Glaser, G. (1988). The nucleotide sequence and characterization of the relA gene of Escherichia coli. J. Biol. Chem. 263, 15699-15704.
Metzger, S., Sarubbi, E., Glaser, G., and Cashel, M. (1989a). Protein sequences encoded by the relA and the spoT genes of Escherichia coli are interrelated. J. Biol. Chem. 264, 9122-9125.
Mittenhuber, G. (2001). Comparative genomics and evolution of genes encoding bacterial (p)ppGpp synthetases/hydrolases (the Rel, RelA and SpoT proteins). J. Mol. Microbiol. Biotechnol. 3, 585-600.
Pedersen, F. S., and Kjeldgaard, N, O. (1977). Analysis of the relA gene product of Escherichia coli. Eur. J. Biochem. 76, 91-97.
Seyfzadeh, M., Keener, J., and Nomura, M. (1993). spoT-dependent accumulation of guanosine tetraphosphate in response to fatty acid starvation in Escherichia coli. Proc. Natl. Acad. Sci. USA 90, 11004-11008.
Takahashi, K., Kasai, K. and Ochi, K. (2004). Identification of the bacterial alarmone guanosine 5′-diphosphate 3′-diphosphate (ppGpp) in plants. Proc. Natl. Acad. Sci. USA 101, 4320-4324.
van der Biezen, E. A., Sun, J., Coleman, M. J., Bibb, M. J., and Jones, J. D. (2000). Arabidopsis RelA/SpoT homologs implicate (p)ppGpp in plant signaling. Proc. Natl. Acad. Sci. USA 97, 3747-3752.
Wendrich, T. M., and Marahiel, M. A. (1997). Cloning and characterization of a relA/spoT homologue from Bacillus subtilis. Mol. Microbiol. 26, 65-79.
Xiao, H., Kalman, M., Ikehara, K., Zemel, S., Glaser, G., and Cashel, M. (1991). Residual guanosine 3′,5′-bispyrophosphate synthetic activity of relA null mutants can be eliminated by spoT null mutations. J. Biol. Chem. 266, 5980-5990.

1.-41. (canceled)

42. A compound represented by the structure of formula (I): 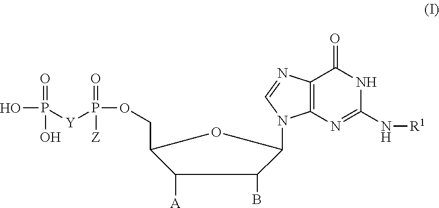
wherein:
A and B are independently selected from the group consisting of:
(a) —H;
(b) —OR²;
(c) 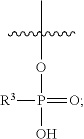
(d) 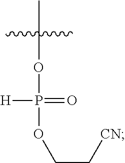
(e) —NR⁴R⁵;
(f) N₃;
(g) 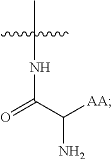
(h) 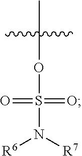
and
(i) 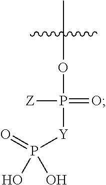
or A and B together represent a moiety selected from:
(a) 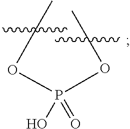
(b) 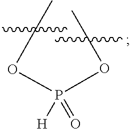
and
(c) 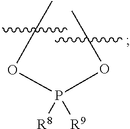
Y is CH₂ or O;
Z is selected from the group consisting of:
(a) OH;
(b) 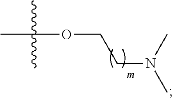
(c) 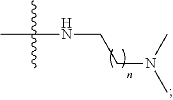
(d) 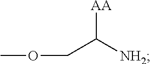
(e) 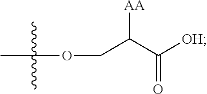
and
(f) 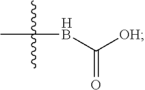
R¹ is H, —COR¹⁰ or an amino protecting group;
R² is H, C₁-C₄ alkyl or a silyl hydroxyl protecting group;
R³ is selected from the group consisting of:
(a) —H;
(b) —OH;
(c) 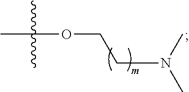
(d) 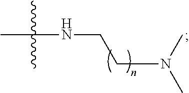
(e) 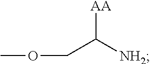
(f) 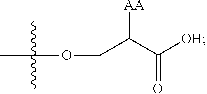
and
(g) 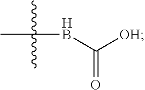
R⁴ and R⁵ are independently H, C₁-C₄ alkyl or an amino protecting group;
R⁶ and R⁷ are independently selected from the group consisting of:
(a) —H;
(b) C₁-C₄ alkyl;
(c) an amino protecting group; and
(d) 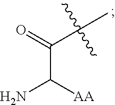
R⁸ and R⁹ are independently selected from the group consisting of:
(a) H;
(b) CH₃;
(c) unsubstituted or substituted aryl, heteroaryl, cycloalkyl or heterocyclyl; and
(d) —(CH₂)ₚ—COOH;
R¹⁰ is H or a C₁-C₄ alkyl;
m, n and p are each independently selected from 0, 1, 2, 3, 4, 5 and 6;
AA represents an amino acid side chain;
with the proviso that:
(a) when Y is O; Z is OH and R¹ is H:
(i) A and B are not both H or OH or 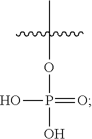
(ii) when one of A and B is H, the other one is not OH;
(iii) when one of A and B is OH, the other one is not OCH₃;
(iv) when A is 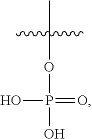
B is not H or OH;
(v) when B is 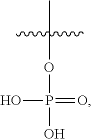
A is not OH;
(vi) when A is 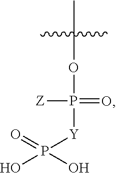
then B is not H or OH;
(vii) when B is 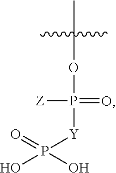
then A is not OH;
(viii) A and B together are not 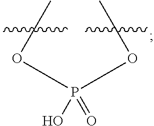
(ix) when A is NH₂, B is not OH; and
(x) when A is N₃, B is not H;
(b) when Y is CH₂; Z is OH and R¹ is H:
(i) when one of A and B is 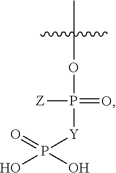
the other is not OH;
(ii) A and B together are not 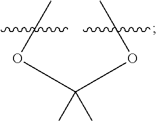
(iii) A and B are not both OH;
(iv) when A is OH, B is not H; and
(v) when B is OH, A is not OCH₃;
including salts, hydrates, solvates, polymorphs, optical isomers, geometrical isomers, enantiomers, diastereomers, complexes and mixtures thereof.

43. The compound according to claim 42, wherein Y is CH₂.

44. The compound according to claim 42, wherein Y is O.

45. The compound according to claim 42, wherein R′ is H.

46. The compound according to claim 42, wherein the compound is selected from the group consisting of: 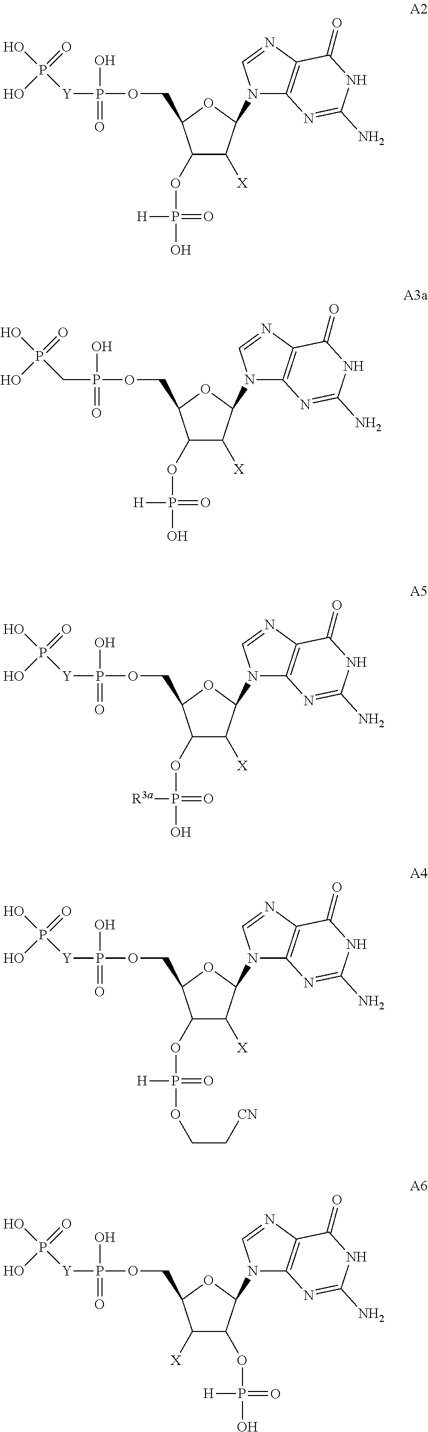 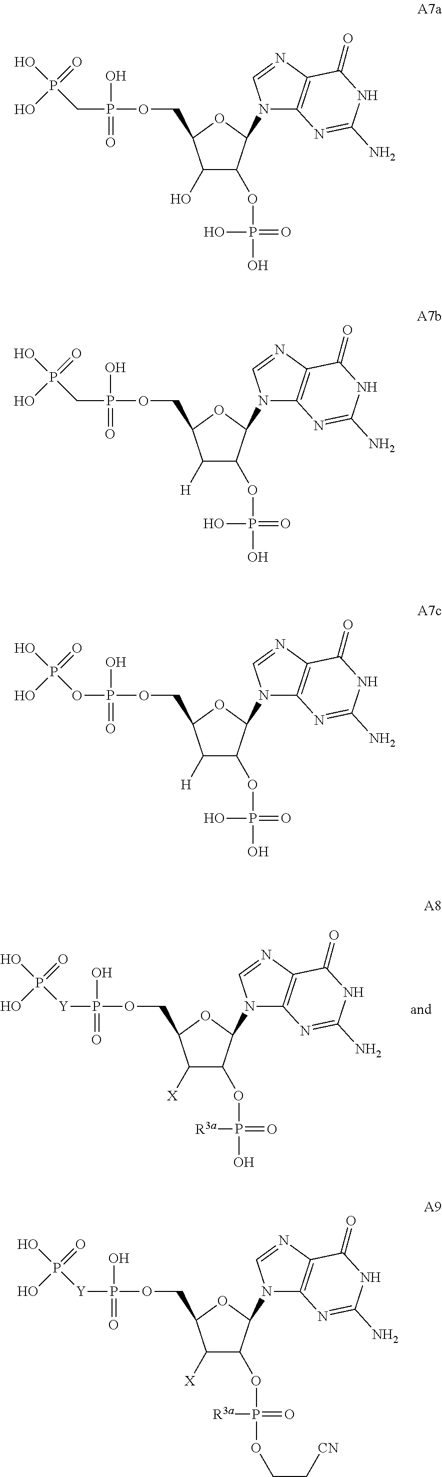
wherein Y is CH₂ or O; X is H or OH; and
R^{3a} is selected from the group consisting of:
(a) 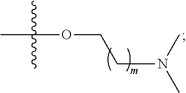
(b) 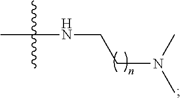
(c) 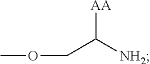
(d) 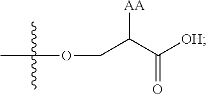
and
(e)

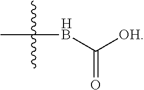

47. The compound according to claim 42, wherein the compound is selected from the group consisting of: 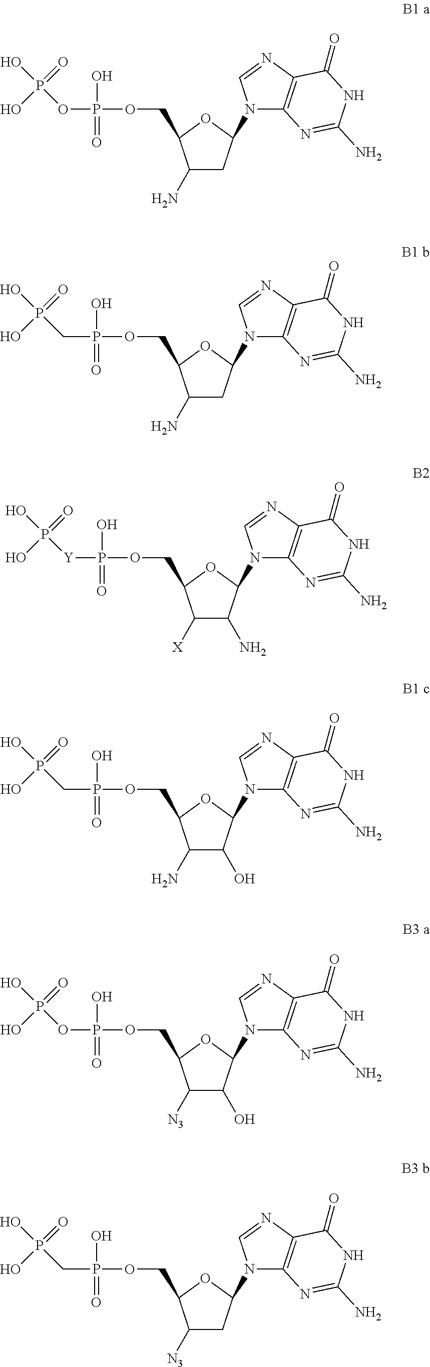 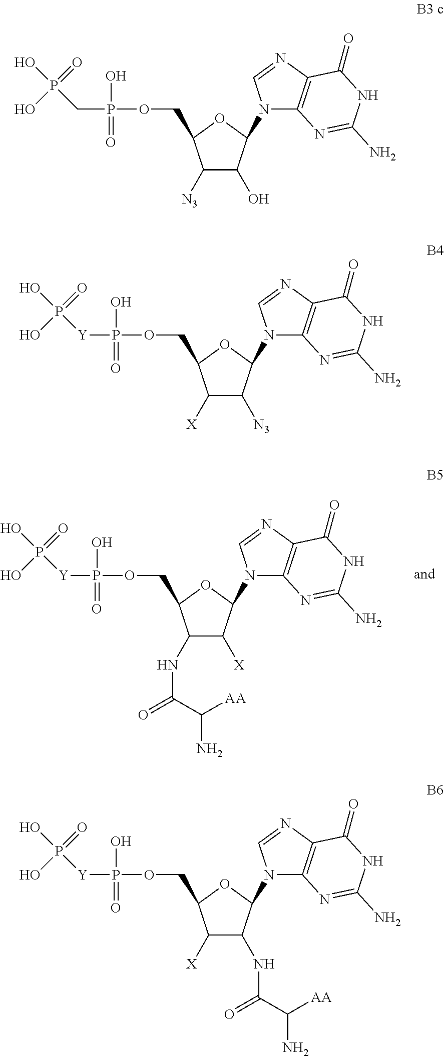
wherein Y is CH₂ or O; X is H or OH; and AA represents an amino acid side chain.

48. The compound according to claim 42, wherein the compound is selected from the group consisting of: 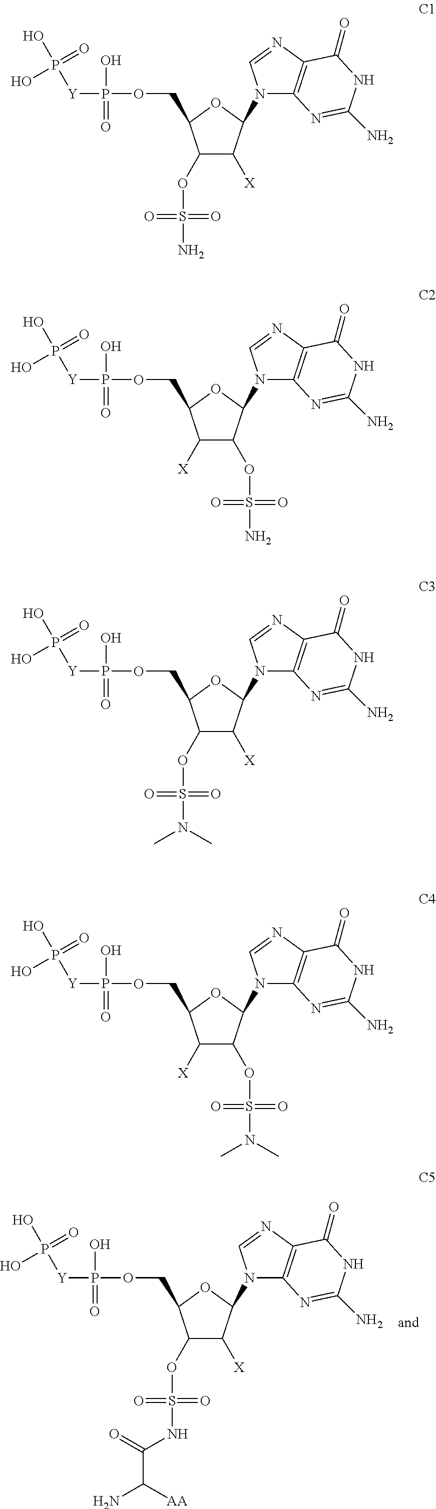 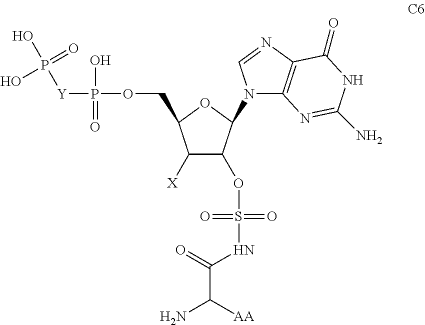
wherein Y is CH₂ or O; X is H or OH; and AA represents an amino acid side chain.

49. The compound according to claim 42, wherein the compound is selected from the group consisting of: 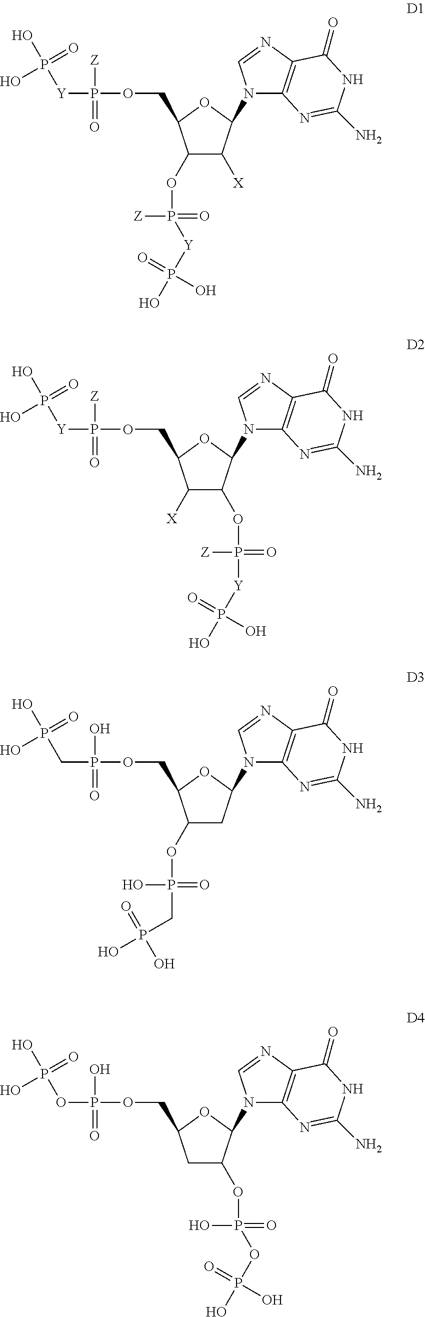 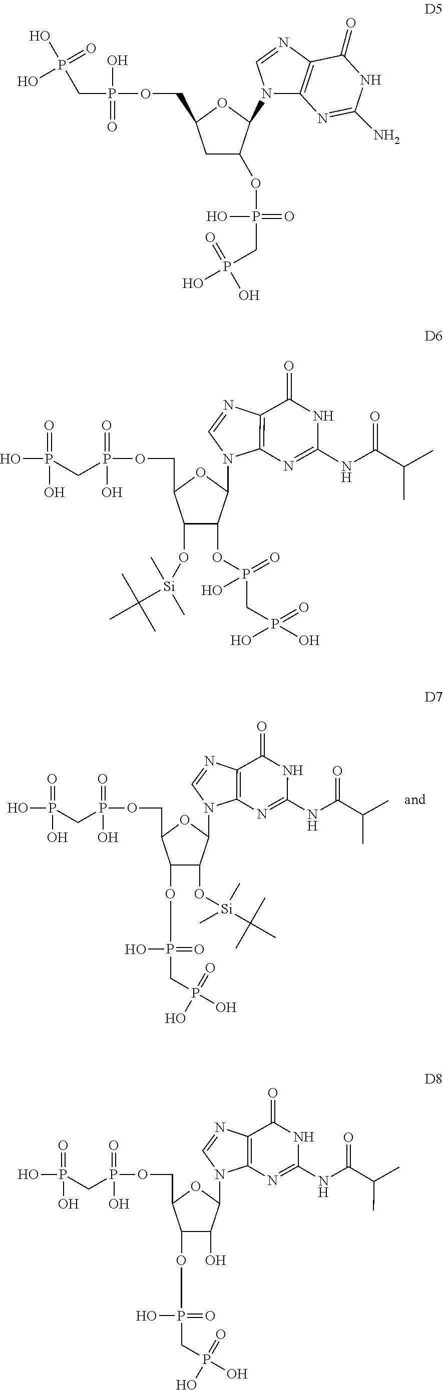
wherein Y is CH₂ or O; X is H or OH; and
Z is selected from the group consisting of:
(a) 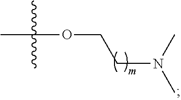
(b) 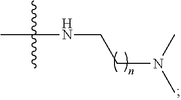
(c) 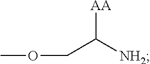
(d) 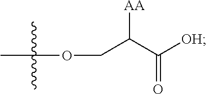
and
(e)

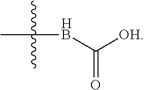

50. The compound according to claim 42, wherein the compound is selected from the group consisting of: 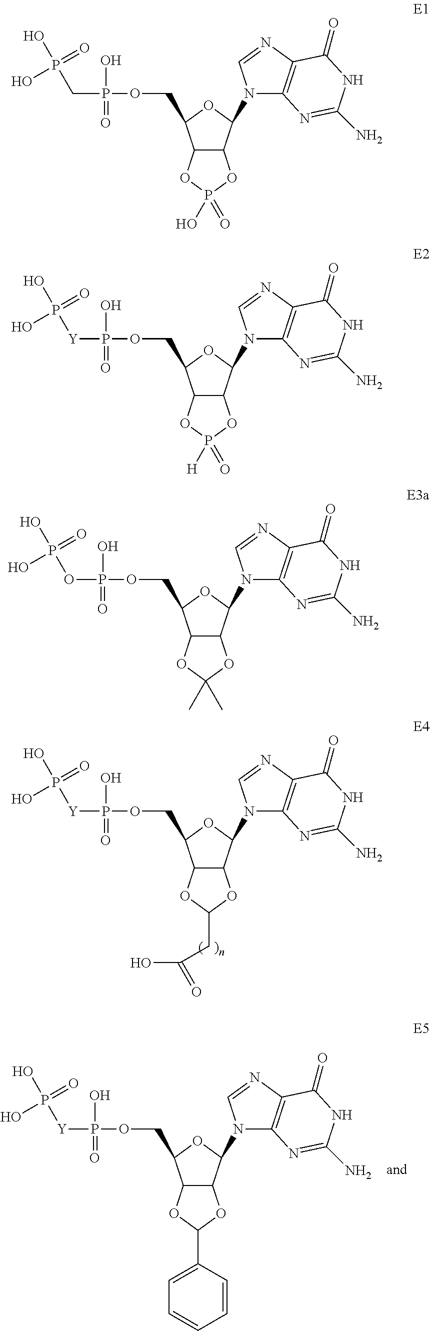 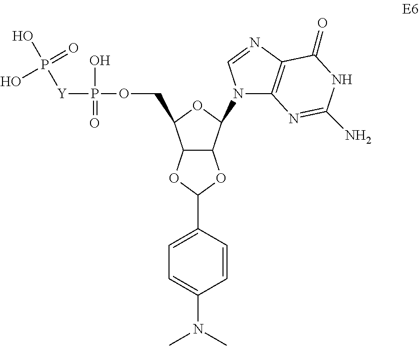
wherein Y is CH₂ or O.

51. The compound according to claim 42, wherein the compound is selected from the group consisting of:

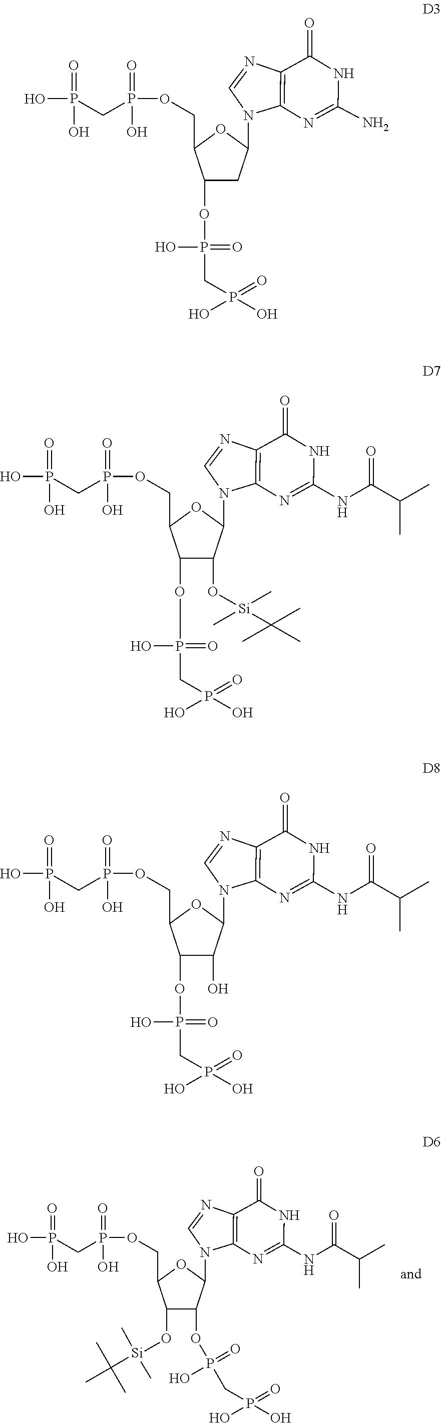

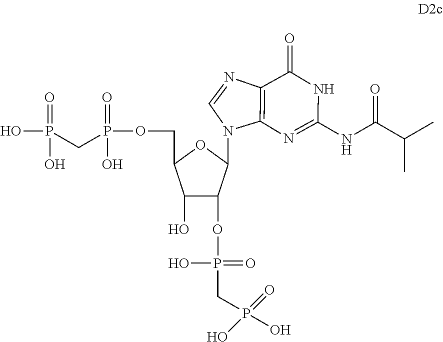

52. An anti-bacterial pharmaceutical composition comprising a therapeutically effective amount of a compound according to claim 42, and a pharmaceutically acceptable carrier or excipient.

53. An anti-bacterial pharmaceutical composition comprising a therapeutically effective amount of a compound of formula (I-a), and a pharmaceutically acceptable carrier or excipient 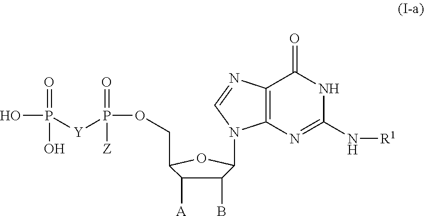
wherein:
A and B are independently selected from the group consisting of:
(a) —H;
(b) —OR²;
(c) 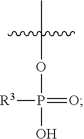
(d) 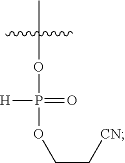
(e) —NR⁴R⁵;
(f) N₃;
(g) 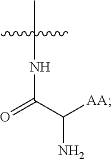
(h) 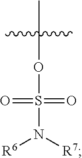
and
(i) 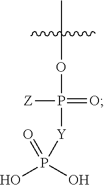
or A and B together represent a moiety selected from:
(a) 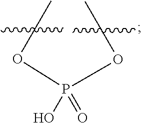
(b) 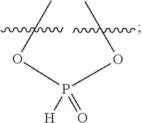
and
(c) 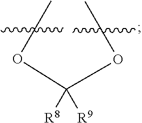
Y is CH₂ or O;
Z is selected from the group consisting of:
(a) OH;
(b) 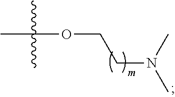
(c) 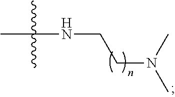
(d) 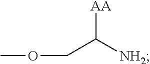
(e) 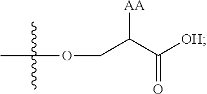
and
(f) 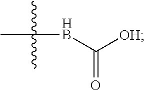
R¹ is H or —COR¹⁶;
R² is H, C₁-C₄ alkyl or a hydroxyl protecting group;
R³ is selected from the group consisting of:
(a) —H;
(b) —OH;
(c) 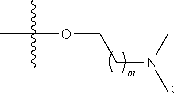
(d) 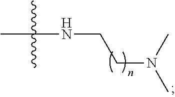
(e) 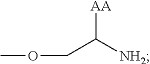
(f) 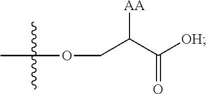
and
(g) 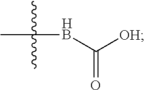
R⁴ and R⁵ are independently H, C₁-C₄ alkyl or an amino protecting group;
R⁶ and R⁷ are independently selected from the group consisting of:
(a) —H;
(b) C₁-C₄ alkyl; and
(c) 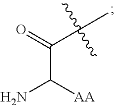
R⁸ and R⁹ are independently selected from the group consisting of:
(a) H;
(b) C₁-C₄ alkyl;
(c) unsubstituted or substituted aryl, heteroaryl, cycloalkyl or heterocyclyl; and
(d) —(CH₂)ₚ—COOH
R¹⁰ is a C₁-C₄ alkyl;
m, n and p are each independently selected from 0, 1, 2, 3, 4, 5 and 6; and
AA represents an amino acid side chain;
with the proviso that when Y is O; Z is OH, R¹ is H and A is 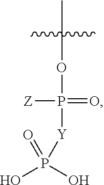
B is not OH;
including salts, hydrates, solvates, polymorphs, optical isomers, geometrical isomers, enantiomers, diastereomers, complexes and mixtures thereof.

54. The composition according to claim 53, wherein Y is CH₂.

55. The composition according to claim 53, wherein Y is O.

56. The composition according to claim 53, wherein R¹ is H.

57. The composition according to claim 12, wherein the compound is selected from the group consisting of: 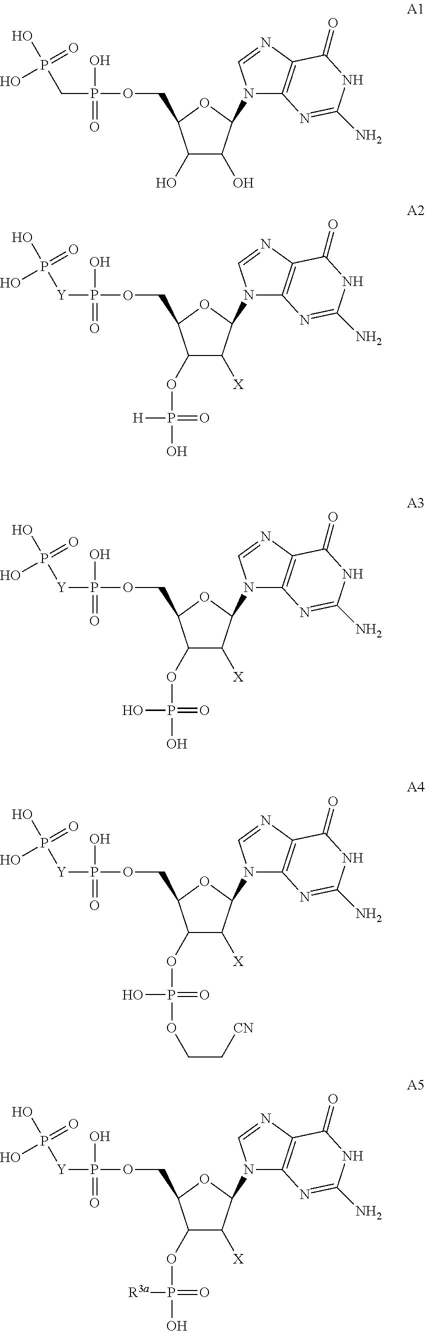 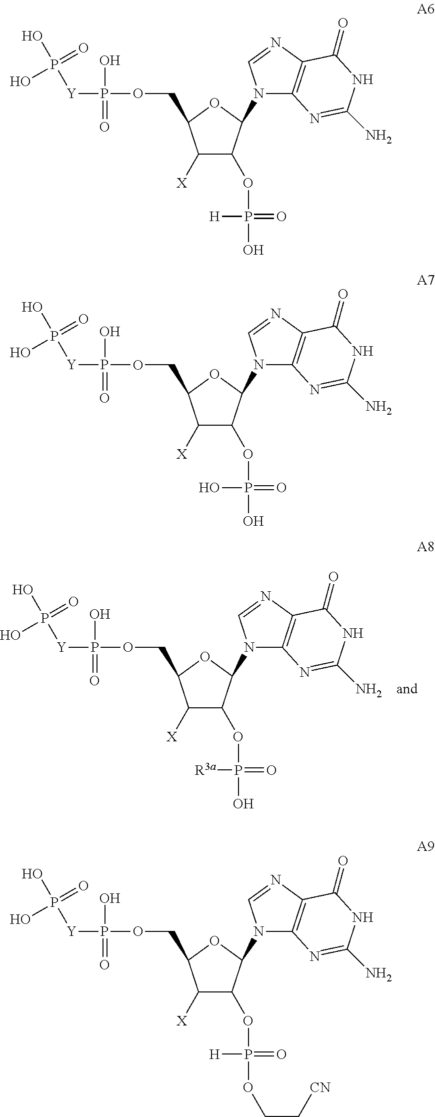
wherein Y is CH₂ or O; X is H or OH; and
R^{3a} is selected from the group consisting of:
(a) 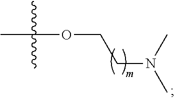
(b) 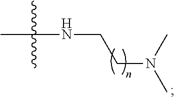
(c) 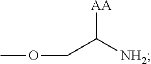
(d) 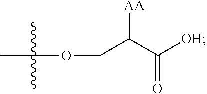
and
(e)

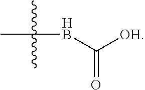

58. The composition according to claim 57, wherein the compound is selected from the group consisting of:

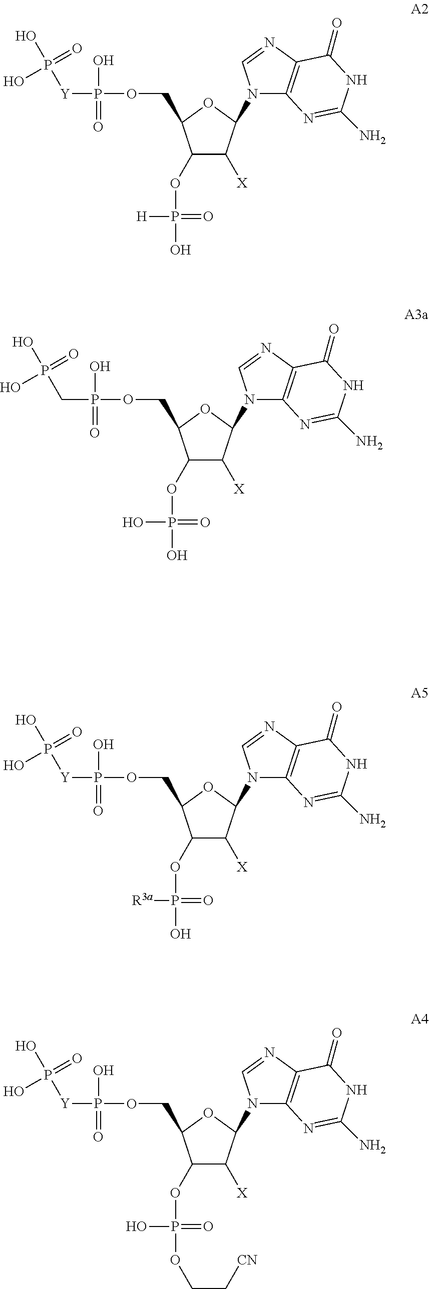

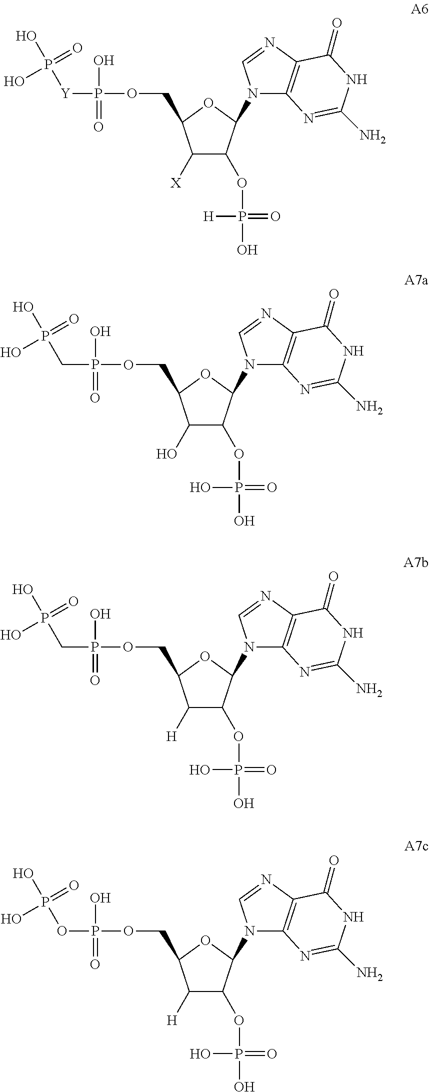

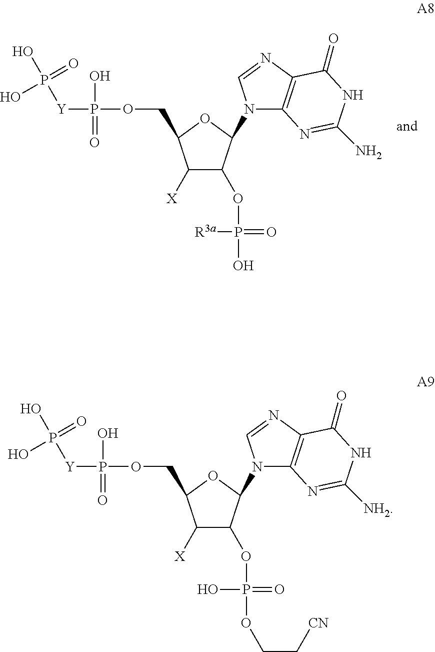

59. The composition according to claim 53, wherein the compound is selected from the group consisting of: 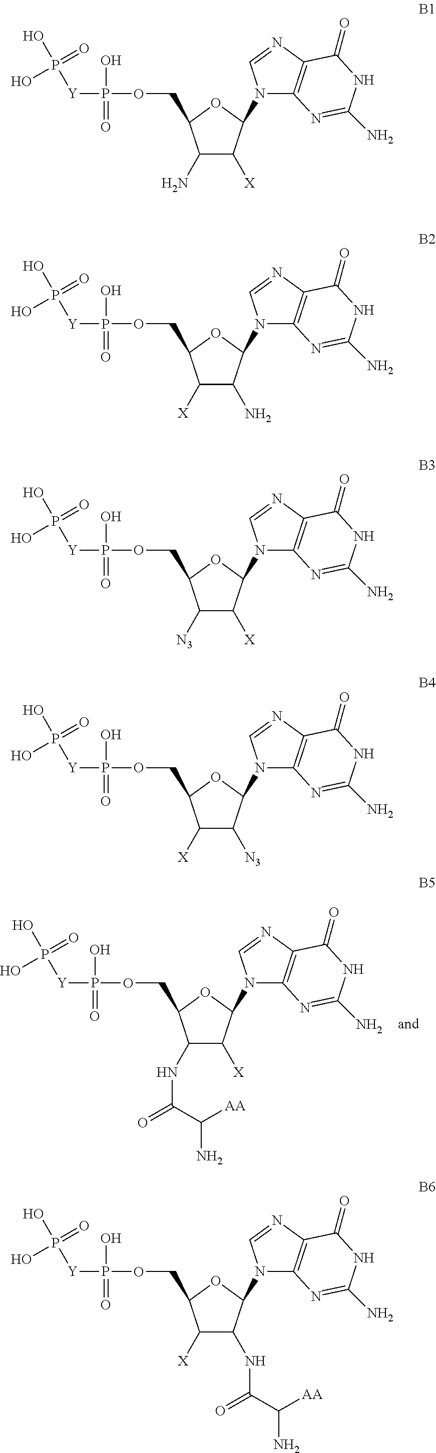
wherein Y is CH₂ or O; X is H or OH; and AA represents an amino acid side chain.

60. The composition according to claim 59, wherein the compound is selected from the group consisting of: 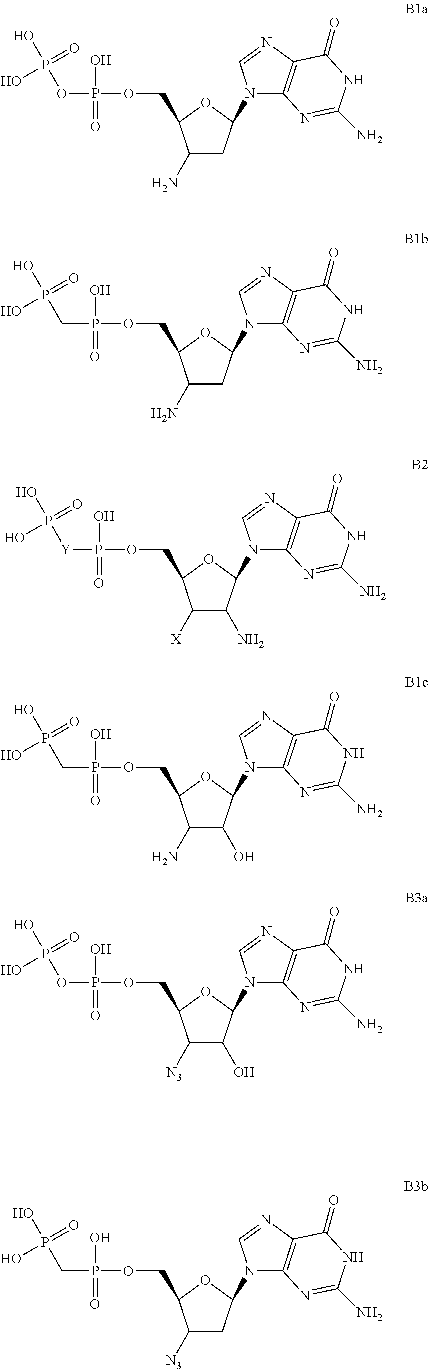 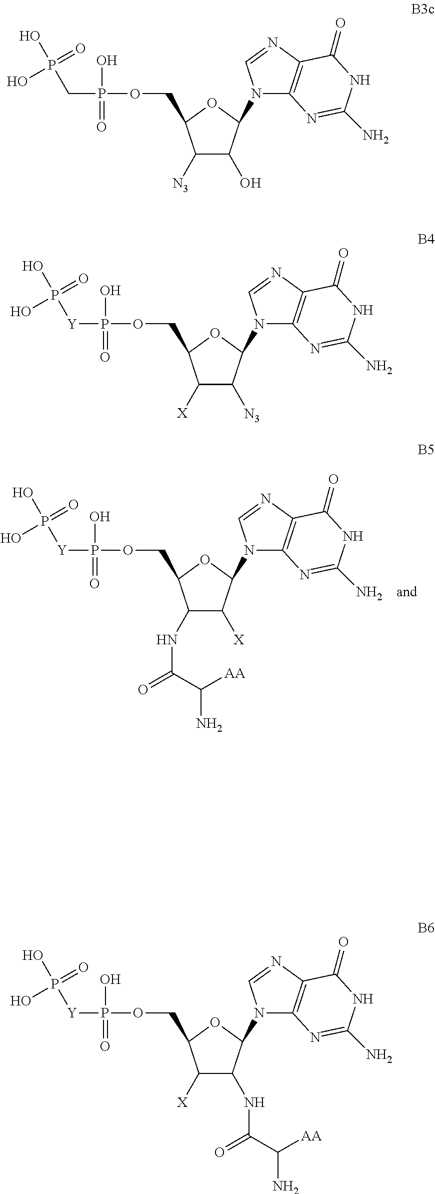
wherein Y is CH₂ or O; X is H or OH; and AA represents an amino acid side chain.

61. The composition according to claim 53, wherein the compound is selected from the group consisting of: 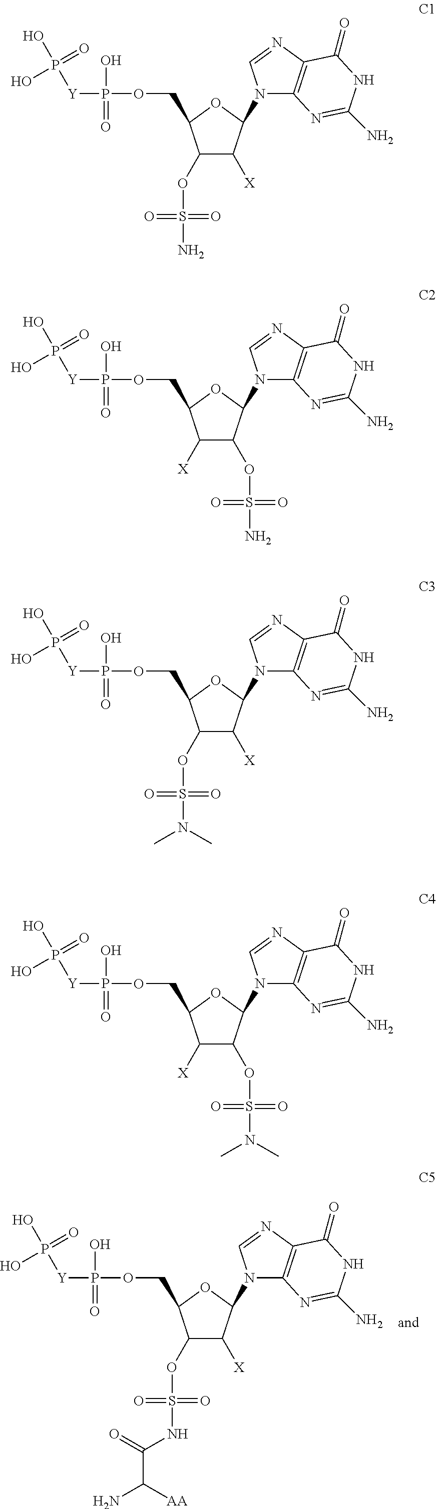 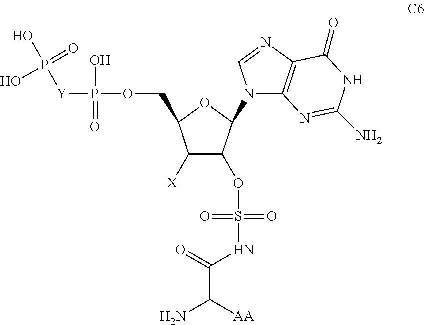
wherein Y is CH₂ or O; X is H or OH; and AA represents an amino acid side chain.

62. The composition according to claim 53, wherein the compound is selected from the group consisting of: 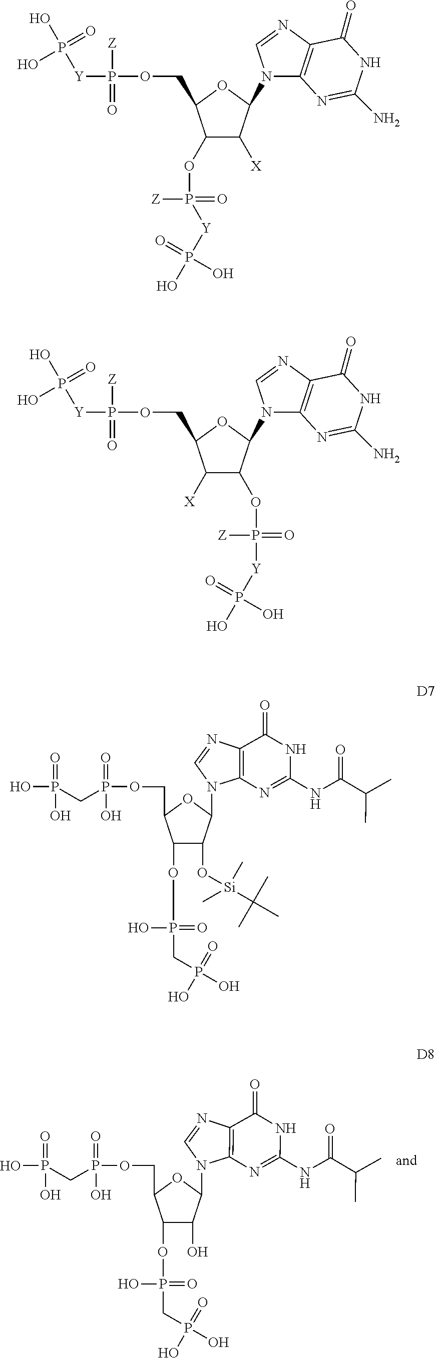 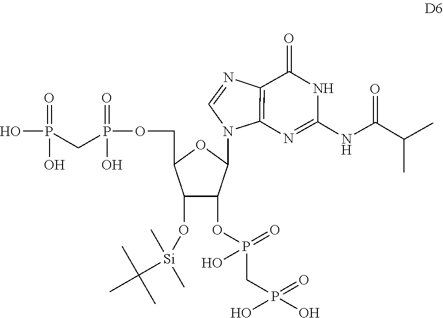
wherein Y is CH₂ or O; X is H or OH; and
Z is selected from the group consisting of:
(a) OH;
(b) 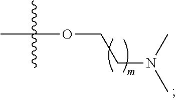
(c) 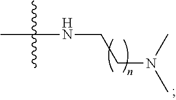
(d) 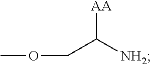
(e) 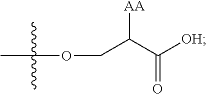
and
(f)

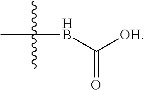

63. The composition according to claim 62, wherein the compound is selected from the group consisting of: 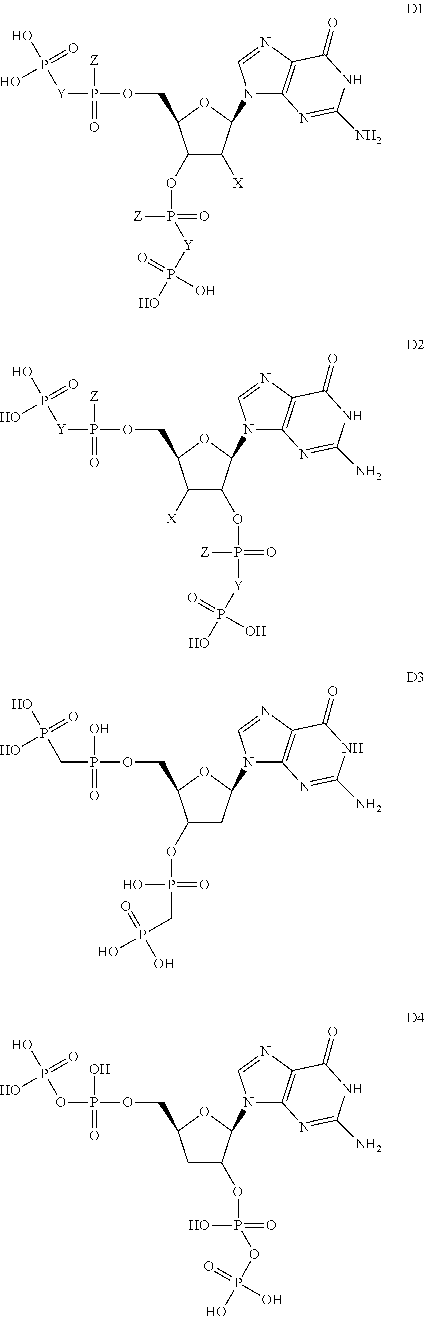 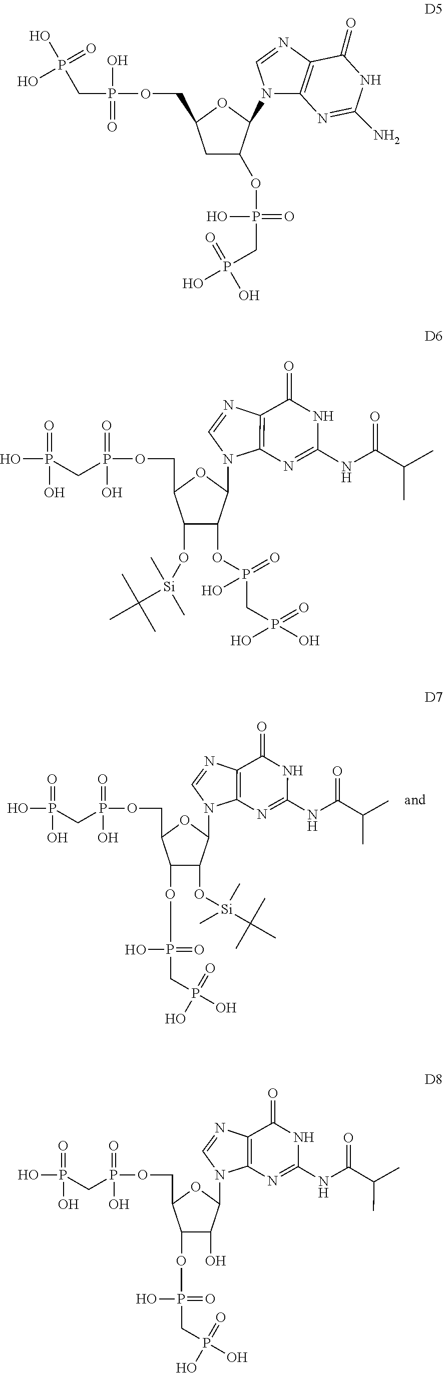
wherein Y is CH₂ or O; X is H or OH; and
Z is selected from the group consisting of:
(a) 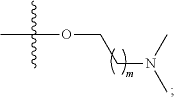
(b) 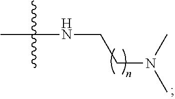
(c) 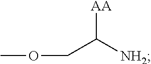
(d) 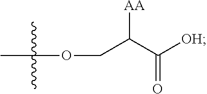
and
(e)

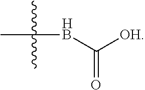

64. The composition according to claim 53, wherein the compound is selected from the group consisting of: 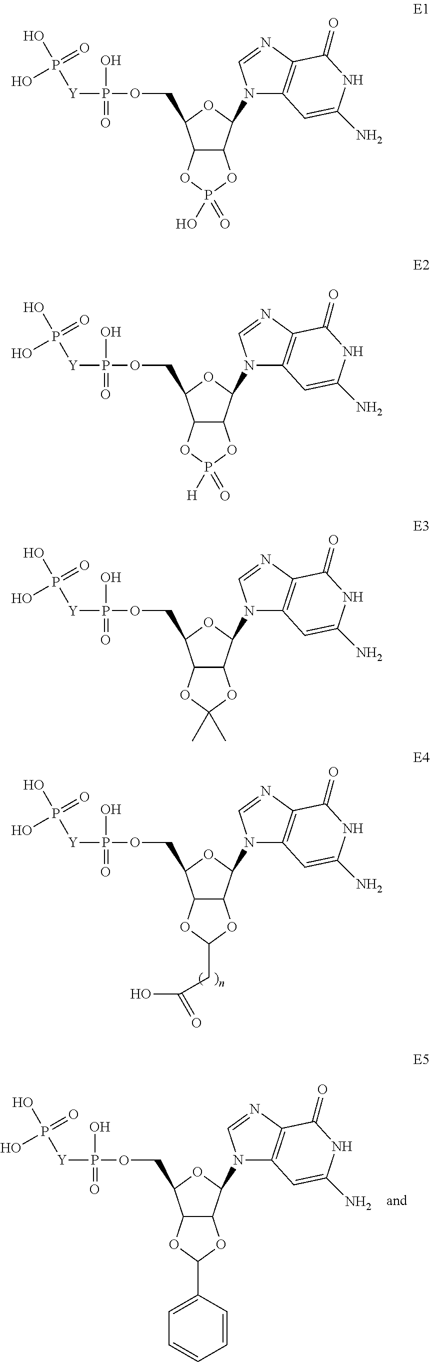 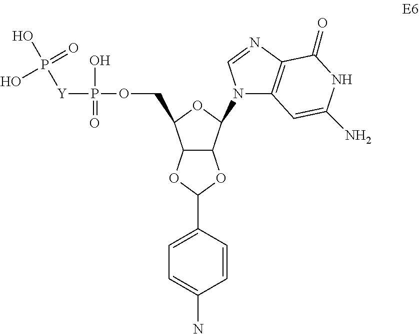
wherein Y is CH₂ or O.

65. The composition according to claim 64, wherein the compound is selected from the group consisting of:

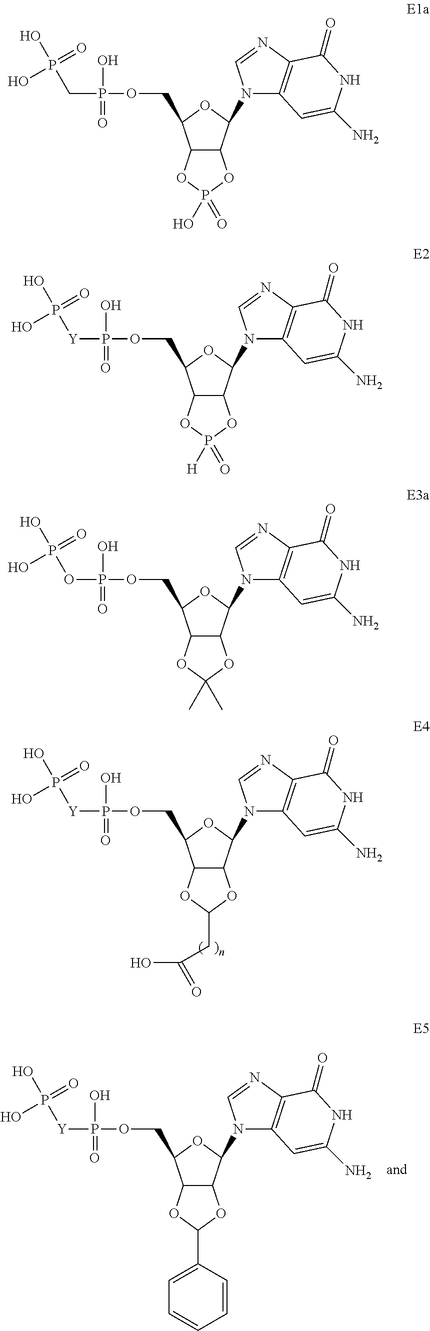

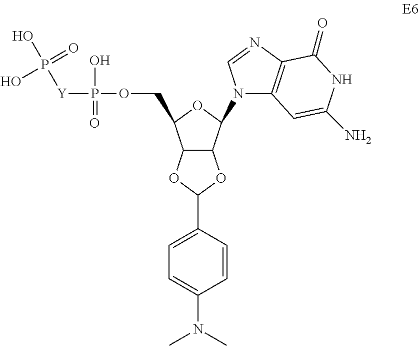

66. The composition according to claim 53, wherein the compound is selected from the group consisting of:

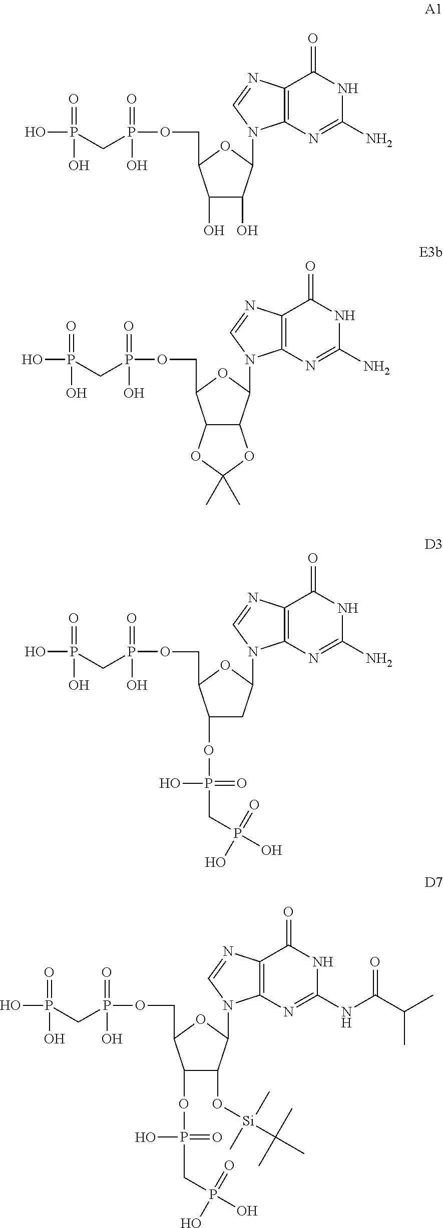

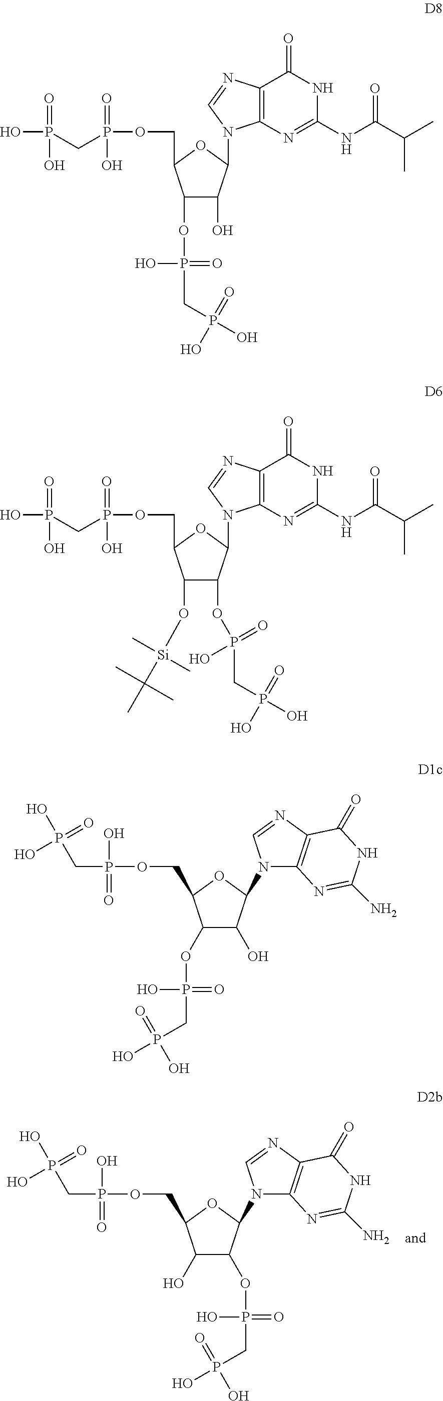

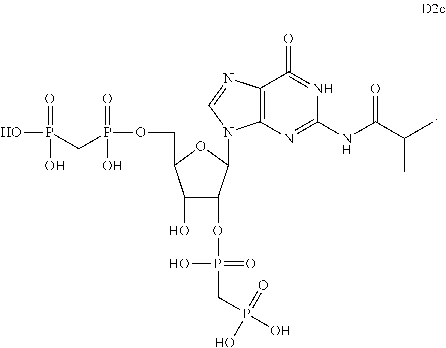

67. A method of combating bacteria, or treating bacterial infections, comprising the step of contacting the bacteria with, or administering to a subject in need thereof a compound according to claim 42.
